(19) United States
(12) Patent Application Publication
(10) Pub. No.: US 2013/0331278 A1
(43) Pub. Date: Dec. 12, 2013

(54) COMPLEX SET OF MIRNAS AS NON-INVASIVE BIOMARKERS FOR PROSTATE DISEASES

(71) Applicants: Andreas Keller, Puttlingen (DE); Markus Beier, Weinheim (DE); Valesca Boisguerin, Mainz (DE); Eckart Meese, Hutschenhausen (DE); Petra Leidinger, Wadern-Nunkirchen (DE)

(72) Inventors: Andreas Keller, Puttlingen (DE); Markus Beier, Weinheim (DE); Valesca Boisguerin, Mainz (DE); Eckart Meese, Hutschenhausen (DE); Petra Leidinger, Wadern-Nunkirchen (DE)

(73) Assignee: febit Holding GmbH, Heidelberg (DE)

(21) Appl. No.: 13/995,951

(22) PCT Filed: Dec. 28, 2011
(86) PCT No.: PCT/EP2011/074169

§ 371 Date: Aug. 21, 2013

(30) Foreign Application Priority Data
Dec. 30, 2010 (PE) 10197448.3

(51) Int. Cl. C12Q 1/68

(52) U.S. Cl. CPC C12Q 1/6886
USPC 506/2; 506/9; 506/16

(57) ABSTRACT

The present invention relates to non-invasive methods, kits and means for diagnosing and/or prognosing of prostate diseases in a body fluid sample from a subject. Further, the present invention relates to set of polynucleotides or sets of primer pairs for detecting sets of miRNAs for diagnosing and/or prognosing of prostate diseases in a body fluid sample from a subject. In addition, the present invention relates to sets of miRNAs for diagnosing and/or prognosing of prostate diseases in a body fluid sample from a subject.

### TECHNICAL FIELD OF THE INVENTION

The present invention relates to a method for diagnosing and/or prognosing of benign prostatic hyperplasia (BPH) based on the determination of expression profiles of sets of miRNAs representative for BPH compared to a reference. Furthermore, the present invention relates to sets of polynucleotides and/or primer pairs for detecting sets of miRNAs for diagnosing and/or prognosing of BPH in a biological sample from a subject. Further, the present invention relates to means for diagnosing and/or prognosing of BPH comprising said sets of primer pairs and/or polynucleotides. In addition, the present invention relates to a kit for diagnosing and/or prognosing of BPH comprising means for determining expression profiles of sets of miRNAs representative for BPH and at least one reference. Further, the present invention relates to use of polynucleotides and/or primer pairs for diagnosing and/or prognosing of BPH in a biological samples of a subject.

### BACKGROUND OF THE INVENTION

Today, biomarkers play a key role in early diagnosis, risk stratification, and therapeutic management of various diseases. While progess in biomarker research has accelerated over the last 5 years, the clinical translation of disease biomarkers as endpoints in disease management and as the foundation for diagnostic products still poses a challenge.

MicroRNAs (miRNAs) are a new class of biomarkers. They represent a group of small noncoding RNAs that regulate gene expression at the posttranslational level by degrading or blocking translation of messenger RNA (mRNA) targets. MiRNAs are important players when it comes to regulate cellular functions and in several diseases, including cancer.

So far, miRNAs have been extensively studied in tissue material. It has been found that miRNAs are expressed in a highly tissue-specific manner. Disease-specific expression of miRNAs have been reported in many human cancers employing primarily tissue material as the miRNA source. In this context miRNAs expression profiles were found to be useful in identifying the tissue of origin for cancers of unknown primary origin.

Since recently it is known that miRNAs are not only present in tissues but also in other body fluid samples, including human blood. Nevertheless, the mechanism why miRNAs are found in body fluids, especially in blood, or their function in these body fluids is not understood yet.

Various miRNA biomarkers found in tissue material have been proposed to be correlated with certain diseases, e.g. cancer. However, there is still a need for novel miRNAs as biomarkers for the detection and/or prediction of these and other types of diseases. Especially desirable are non-invasive biomarkers, that allow for quick, easy and cost-effective diagnosis/prognosis which cause only minimal stress for the patient eliminating the need for surgical intervention

Particularly, the potential role of miRNAs as non-invasive biomarkers for the diagnosis and/or prognosis of BPH has not been systematically evaluated yet. In addition, many of the miRNA biomarkers presently available for diagnosing and/or prognosing of diseases have shortcomings such as reduced sensitivity, not sufficient specificity or do not allow timely diagnosis or represent invasive biomarkers. Accordingly, there is still a need for novel and efficient miRNAs or sets of miRNAs as markers, effective methods and kits for the non-invasive diagnosis and/or prognosis of diseases such as BPH.

The inventors of the present invention assessed for the first time the expression of miRNAs on a whole-genome level in subjects with BPH as non-invasive biomarkers from body fluids, preferably in blood. They surprisingly found that miRNAs are significantly dysregulated in blood of BPH subjects in comparison to healthy controls or in comparison to prostate cancer subjects. Thus, miRNAs are appropriated non-invasive biomarkers for diagnosing and/or prognosing of BPH. This finding is surprising, since there is nearly no overlap of the miRNA biomarkers found in blood and the miRNA biomarkers found in tissue material representing the origin of the disease. The inventors of the present invention surprisingly found miRNA biomarkers in body fluids, especially in blood, that have not been found to be correlated to BPH when tissues material was used for this kind of analysis. Therefore, the inventors of the invention identified for the first time miRNAs as non-invasive surrogate biomarkers for diagnosis and/or prognosis of BPH. The inventors of the present invention identified single miRNAs which predict BPH with high specificity, sensitivity and accuracy. The inventors of the present invention also pursued a multiple biomarker strategy, thus implementing sets of miRNA biomarkers for diagnosing and/or prognosing of BPH leading to added specificity, sensitivity, accuracy and predictive power, thereby circumventing the limitations of single biomarker. In detail, by using a machine learning algorithms, they identified unique sets of miRNAs (miRNA signatures) that allow for non-invasive diagnosis of BPH with even higher power, indicating that sets of miRNAs (miRNA signatures) derived from a body fluid sample, such as blood from a subject (e.g. human) can be used as novel non-invasive biomarkers.

### SUMMARY OF THE INVENTION

In a first aspect, the invention provides a method for diagnosing and/or prognosing of BPH comprising the steps of:
(i) determining an expression profile of a set comprising at least two miRNAs representative for BPH in a body fluid sample from a subject, and
(ii) comparing said expression profile to a reference, wherein the comparison of said expression profile to said reference allows for the diagnosis and/or prognosis of BPH,

In a second aspect, the invention provides a set comprising polynucleotides for detecting a set comprising at least two miRNAs for diagnosing and/or prognosing of BPH in a body fluid sample from a subject.

In a third aspect, the invention provides a use of a set of polynucleotides according to the second aspect of the invention for diagnosing and/or prognosing BPH in a subject

In a fourth aspect, the invention provides a set of primer pairs for determining the expression level of a set of miRNAs in a body fluid sample of a subject suffering or suspected of suffering from BPH.

In a fifth aspect, the invention provides a use of set of primer pairs according to the fourth aspect of the invention for diagnosing and/or prognosing BPH in a subject

In a sixth aspect, the invention provides means for diagnosing and/or prognosing of BPH in a body fluid sample of a subject comprising:
(i) a set of at least two polynucleotides according to the second aspect of the invention or
(ii) a set of primer pairs according the fourth aspect of the invention.

In a seventh aspect, the invention provides a kit for diagnosing and/or prognosing of BPH comprising
(i) means for determining an expression profile of a set comprising at least two miRNAs representative for BPH in a body fluid sample from a subject, and
(ii) at least one reference.

In an eighth aspect, the invention provides a set of miRNAs in a body fluid sample isolated from a subject for diagnosing and/or prognosing of BPH.

In a ninth aspect, the invention provides a use of a set of miRNAs according to the eighth aspect of the invention for diagnosing and/or prognosing of BPH in a subject, This summary of the invention does not necessarily describe all features of the invention.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

To practice the present invention, unless otherwise indicated, conventional methods of chemistry, biochemistry, and recombinant DNA techniques are employed which are explained in the literature in the field (cf., e.g., Molecular Cloning: A Laboratory Manual, 2^{nd} Edition, J. Sambrook et al. eds., Cold Spring Harbor Laboratory Press, Cold Spring Harbor 1989).

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used in this specification and in the appended claims, the singular forms “a”, “an”, and “the” include plural referents, unless the content clearly dictates otherwise. For example, the term “a test compound” also includes “test compounds”.

The terms “microRNA” or “miRNA” refer to single-stranded RNA molecules of at least 10 nucleotides and of not more than 35 nucleotides covalently linked together. Preferably, the polynucleotides of the present invention are molecules of 10 to 33 nucleotides or 15 to 30 nucleotides in length, more preferably of 17 to 27 nucleotides or 18 to 26 nucleotides in length, i.e. 10, 11, 12, 13, 14, 15, 16, 17, 18, 19, 20, 21, 22, 23, 24, 25, 26, 27, 28, 29, 30, 31, 32, 33, 34, or 35 nucleotides in length, not including optionally labels and/or elongated sequences (e.g. biotin stretches). The miRNAs regulate gene expression and are encoded by genes from whose DNA they are transcribed but miRNAs are not translated into protein (i.e. miRNAs are noncoding RNAs). The genes encoding miRNAs are longer than the processed mature miRNA molecules. The miRNAs are first transcribed as primary transcripts or pri-miRNAs with a cap and poly-A tail and processed to short, 70 nucleotide stem-loop structures known as pre-miRNAs in the cell nucleus. This processing is performed in animals by a protein complex known as the Microprocessor complex consisting of the nuclease Drosha and the double-stranded RNA binding protein Pasha. These pre-miRNAs are then processed to mature miRNAs in the cytoplasm by interaction with the endonuclease Dicer, which also initiates the formation of the RNA-induced silencing complex (RISC). When Dicer cleaves the pre-miRNA stem-loop, two complementary short RNA molecules are formed, but only one is integrated into the RISC. This strand is known as the guide strand and is selected by the argonaute protein, the catalytically active RNase in the RISC, on the basis of the stability of the 5′ end. The remaining strand, known as the miRNA*, anti-guide (anti-strand), or passenger strand, is degraded as a RISC substrate. Therefore, the miRNA*s are derived from the same hairpin structure like the “normal” miRNAs. So if the “normal” miRNA is then later called the “mature miRNA” or “guide strand”, the miRNA* is the “anti-guide strand” or “passenger strand”.

The terms “microRNA*” or “miRNA*” refer to single-stranded RNA molecules of at least 10 nucleotides and of not more than 35 nucleotides covalently linked together. Preferably, the polynucleotides of the present invention are molecules of 10 to 33 nucleotides or 15 to 30 nucleotides in length, more preferably of 17 to 27 nucleotides or 18 to 26 nucleotides in length, i.e. 10, 11, 12, 13, 14, 15, 16, 17, 18, 19, 20, 21, 22, 23, 24, 25, 26, 27, 28, 29, 30, 31, 32, 33, 34, or 35 nucleotides in length, not including optionally labels and/or elongated sequences (e.g. biotin stretches). The “miRNA*s”, also known as the “anti-guide strands” or “passenger strands”, are mostly complementary to the “mature miRNAs” or “guide strands”, but have usually single-stranded overhangs on each end. There are usually one or more mispairs and there are sometimes extra or missing bases causing single-stranded “bubbles”. The miRNA*s are likely to act in a regulatory fashion as the miRNAs (see also above). In the context of the present invention, the terms “miRNA” and “miRNA*” are interchangeable used. The present invention encompasses (target) miRNAs which are dysregulated in biological samples such as blood or tissue of BPH patients in comparison to healthy controls. Said (target) miRNAs are preferably selected from the group consisting of SEQ ID NO: 1 to 182 and 352-353.

The term “miRBase” refers to a well established repository of validated miRNAs. The miRBase (www.mirbase.org) is a searchable database of published miRNA sequences and annotation. Each entry in the miRBase Sequence database represents a predicted hairpin portion of a miRNA transcript (termed mir in the database), with information on the location and sequence of the mature miRNA sequence (termed miR). Both hairpin and mature sequences are available for searching and browsing, and entries can also be retrieved by name, keyword, references and annotation. All sequence and annotation data are also available for download.

As used herein, the term “nucleotides” refers to structural components, or building blocks, of DNA and RNA. Nucleotides consist of a base (one of four chemicals: adenine, thymine, guanine, and cytosine) plus a molecule of sugar and one of phosphoric acid. The term “nucleosides” refers to glycosylamine consisting of a nucleobase (often referred to simply base) bound to a ribose or deoxyribose sugar. Examples of nucleosides include cytidine, uridine, adenosine, guanosine, thymidine and inosine. Nucleosides can be phosphorylated by specific kinases in the cell on the sugar's primary alcohol group (—CH2-OH), producing nucleotides, which are the molecular building blocks of DNA and RNA.

The term “polynucleotide”, as used herein, means a molecule of at least 10 nucleotides and of not more than 35 nucleotides covalently linked together. Preferably, the polynucleotides of the present invention are molecules of 10 to 33 nucleotides or 15 to 30 nucleotides in length, more preferably of 17 to 27 nucleotides or 18 to 26 nucleotides in length, i.e. 10, 11, 12, 13, 14, 15, 16, 17, 18, 19, 20, 21, 22, 23, 24, 25, 26, 27, 28, 29, 30, 31, 32, 33, 34, or 35 nucleotides in length, not including optionally spacer elements and/or elongation elements described below. The depiction of a single strand of a polynucleotide also defines the sequence of the complementary strand. Polynucleotides may be single stranded or double stranded, or may contain portions of both double stranded and single stranded sequences. The term “polynucleotide” means a polymer of deoxyribonucleotide or ribonucleotide bases and includes DNA and RNA molecules, both sense and anti-sense strands. In detail, the polynucleotide may be DNA, both cDNA and genomic DNA, RNA, cRNA or a hybrid, where the polynucleotide sequence may contain combinations of deoxyribonucleotide or ribonucleotide bases, and combinations of bases including uracil, adenine, thymine, cytosine, guanine, inosine, xanthine, hypoxanthine, isocytosine and isoguanine Polynucleotides may be obtained by chemical synthesis methods or by recombinant methods.

In the context of the present invention, a polynucleotide as a single polynucleotide strand provides a probe (e.g. miRNA capture probe) that is capable of binding to, hybridizing with, or detecting a target of complementary sequence, such as a nucleotide sequence of a miRNA or miRNA*, through one or more types of chemical bonds, usually through complementary base pairing, usually through hydrogen bond formation. Polynucleotides in their function as probes may bind target sequences, such as nucleotide sequences of miRNAs or miRNAs*, lacking complete complementarity with the polynucleotide sequences depending upon the stringency of the hybridization condition. There may be any number of base pair mismatches which will interfere with hybridization between the target sequence, such as a nucleotide sequence of a miRNA or miRNA*, and the single stranded polynucleotide described herein. However, if the number of mutations is so great that no hybridization can occur under even the least stringent hybridization conditions, the sequences are no complementary sequences. The present invention encompasses polynucleotides in form of single polynucleotide strands as probes for binding to, hybridizing with or detecting complementary sequences of (target) miRNAs for diagnosing and/or prognosing of BPH. Said (target) miRNAs are preferably selected from the group consisting of SEQ ID NO: 1 to 182 and 352-353.

Because of the conservation of miRNAs among species, for example between humans and other mammals, e.g. animals such as mice, monkey or rat, the polynucleotide(s) of the invention may not only be suitable for detecting a miRNA(s) of a specific species, e.g. a human miRNA, but may also be suitable for detecting the respective miRNA orthologue(s) in another species, e.g. in another mammal, e.g. animal such as mouse or rat.

The term “antisense”, as used herein, refers to nucleotide sequences which are complementary to a specific DNA or RNA sequence. The term “antisense strand” is used in reference to a nucleic acid strand that is complementary to the “sense” strand.

The term “label”, as used herein, means a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and other entities which can be made detectable. A label may be incorporated into nucleic acids at any position, e.g. at the 3′ or 5′ end or internally. The polynucleotide for detecting a miRNA (polynucleotide probe) and/or the miRNA itself may be labeled. For detection purposes, the miRNA(s) or miRNA*(s) may be employed unlabeled, directly labeled, or indirectly labeled, such as with biotin to which a streptavidin complex may later bind.

The term “stringent hybridization conditions”, as used herein, means conditions under which a first nucleotide sequence (e.g. polynucleotide in its function as a probe for detecting a miRNA or miRNA*) will hybridize to a second nucleotide sequence (e.g. target sequence such as nucleotide sequence of a miRNA or miRNA*), such as in a complex mixture of nucleotide sequences. Stringent conditions are sequence-dependent and will be different in different circumstances. Stringent conditions may be selected to be about 5 to 10° C. lower than the thermal melting point (Tm) for the specific sequence at a defined ionic strength, pH. The Tm may be the temperature (under defined ionic strength, pH, and nucleic acid concentration) at which 50% of the probes complementary to the target hybridize to the target sequence at equilibrium (as the target sequences are present in excess, at Tm, 50% of the probes are occupied at equilibrium). Stringent conditions may be those in which the salt concentration is less than about 1.0 M sodium ion, such as about 0.01 to 1.0 M sodium ion concentration (or other salts) at pH 7.0 to 8.3 and the temperature is at least about 20° C. for short probes (e.g. about 10-35 nucleotides) and up to 60° C. for long probes (e.g. greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.; or 6×SSPE, 10% formamide, 0.01%, Tween 20, 0.1×TE buffer, 0.5 mg/ml BSA, 0.1 mg/ml herring sperm DNA, incubating at 42° C. with wash in 05×SSPE and 6×SSPE at 45° C.

The term “sensitivity”, as used herein, means a statistical measure of how well a binary classification test correctly identifies a condition, for example how frequently it correctly classifies a heart and cardiovascular system disease into the correct type out of two or more possible types (e.g. heart and cardiovascular system disease type and healthy type). The sensitivity for class A is the proportion of cases that are determined to belong to class “A” by the test out of the cases that are in class “A”. A theoretical, optimal prediction can achieve 100% sensitivity (i.e. predict all patients from the sick group as sick).

The term “specificity”, as used herein, means a statistical measure of how well a binary classification test correctly identifies a condition, for example how frequently it correctly classifies a heart and cardiovascular system disease into the correct type out of two or more possible types. The specificity for class A is the proportion of cases that are determined to belong to class “not A” by the test out of the cases that are in class “not A”. A theoretical, optimal prediction can achieve 100% specificity (i.e. not predict anyone from the healthy group as sick).

The term “accuracy”, as used herein, means a statistical measure for the correctness of classification or identification of sample types. The accuracy is the proportion of true results (both true positives and true negatives).

The term “biological sample”, as used in the context of the present invention, refers to any biological sample containing miRNA(s). Said biological sample may be a biological fluid, tissue, cell(s) or mixtures thereof. For example, biological samples encompassed by the present invention are body fluids, tissue (e.g. section or explant) samples, cell culture samples, cell colony samples, single cell samples, collection of single cell samples, blood samples (e.g. whole blood or a blood fraction such as serum or plasma or blood cell fractions such as red blood cells, platelets, white blood cells, T-cells, NK-cells, regulatory T-cells, B-cells, granulocytes etc.), urine samples, or samples from other peripheral sources. Said biological samples may be mixed or pooled, e.g. a biological sample may be a mixture of blood and urine samples. A “biological sample” may be provided by removing cell(s), cell colonies, an explant, or a section from a subject suspected to be affected by BPH, but may also be provided by using a previously isolated sample. For example, a tissue sample may be removed from a subject suspected to be affected by BPHs by conventional biopsy techniques or a blood sample may be taken from a subject suspected to be affected by BPH by conventional blood collection techniques. The biological sample, e.g. tissue or blood sample, may be obtained from a subject suspected to be affected by BPH prior to initiation of the therapeutic treatment, during the therapeutic treatment and/or after the therapeutic treatment.

The term “body fluid sample”, as used in the context of the present invention, refers to liquids originating from the body of a subject. Said body fluid samples include, but are not limited to, blood, urine, sputum, breast milk, cerebrospinal fluid, cerumen (earwax), endolymph, perilymph, gastric juice, mucus, peritoneal fluid, pleural fluid, saliva, sebum (skin oil), semen, sweat, tears, vaginal secretion, vomit including components or fractions thereof. Said body fluid samples may be mixed or pooled, e.g. a body fluid sample may be a mixture of blood and urine samples or blood and tissue material. A “body fluid sample” may be provided by removing a body liquid from a subject, but may also be provided by using previously isolated sample material.

Preferably, the body fluid sample from a subject (e.g. human or animal) has a volume of between 0.1 and 20 ml, more preferably of between 0.5 and 10 ml, more preferably between 1 and 8 ml and most preferably between 2 and 5 ml, i.e. 0.1, 0.2, 0.3, 0.4, 0.5, 0.6, 0.7, 0.8, 0.9, 1, 2, 2.5, 3, 4, 5, 6, 7, 8, 9, 10, 11, 12, 13, 14, 15, 16, 17, 18, 19, or 20 ml.

In the context of the present invention said “body fluid sample” allows for a non-invasive diagnosis/and or prognosis of a subject.

The term “blood sample”, as used in the context of the present invention, refers to a blood sample originating from a subject. The “blood sample” may be derived by removing blood from a subject by conventional blood collecting techniques, but may also be provided by using previously isolated and/or stored blood samples. For example a blood sample may be whole blood, plasma, serum, PBMC (peripheral blood mononuclear cells), blood cellular fractions including red blood cells (erythrocytes), white blood cells (leukocytes) or subfractions thereof (e.g. T-cells, NK-cells, regulatory T-cells, B-cells, granulocytes), platelets (thrombocytes), or blood collected in blood collection tubes (e.g. EDTA-, heparin-, citrate-, PAXgene-, Tempus-tubes) including components or fractions thereof. For example, a blood sample may be taken from a subject suspected to be affected or to be suspected to be affected by BPH, prior to initiation of a therapeutic treatment, during the therapeutic treatment and/or after the therapeutic treatment.

Preferably, the blood sample from a subject (e.g. human or animal) has a volume of between 0.1 and 20 ml, more preferably of between 0.5 and 10 ml, more preferably between 1 and 8 ml and most preferably between 2 and 5 ml, i.e. 0.1, 0.2, 0.3, 0.4, 0.5, 0.6, 0.7, 0.8, 0.9, 1, 2, 2.5, 3, 4, 5, 6, 7, 8, 9, 10, 11, 12, 13, 14, 15, 16, 17, 18, 19, or 20 ml.

In the context of the present invention said “body fluid sample” allows for a non-invasive diagnosis/and or prognosis of a subject.

Preferably, when the blood sample is collected from the subject the RNA-fraction, especially the miRNA fraction, is guarded against degradation. For this purpose special collection tubes (e.g. PAXgene RNA tubes from Preanalytix, Tempus Blood RNA tubes from Applied Biosystems) or additives (e.g. RNAlater from Ambion, RNAsin from Promega) that stabilize the RNA fraction and/or the miRNA fraction are employed.

The biological sample, preferably the body fluid sample may be from a subject (e.g. human or mammal) that has been therapeutically treated or that has not been therapeutically treated. In one embodiment, the therapeutical treatment is monitored on the basis of the detection of the miRNA or set of miRNAs by the polynucleotide or set of polynucleotides of the invention. It is also preferred that total RNA or a subfraction thereof, isolated (e.g. extracted) from a biological sample of a subject (e.g. human or animal), is used for detecting the miRNA or set of miRNAs by the polynucleotide or set of polynucleotides or primer pairs of the invention.

The term “non-invasive”, as used in the context of the present invention, refers to methods for obtaining a biological sample, particularly a body fluid sample, without the need for an invasive surgical intervention or invasive medical procedure. In the context of the present invention, a blood drawn represents a non-invasive procedure, therefore a blood-based test (utilizing blood or fractions thereof) is a non-invasive test. Other body fluid samples for non-invasive tests are e.g. urine, sputum, tears, mothers mild, cerumen, sweat, saliva, vaginal secretion, vomit, etc.

The term “minimal invasive”, as used in the context of the present invention, refers to methods for obtaining a biological sample, particularly a body fluid sample, with a minimal need for an invasive surgical intervention or invasive medical procedure.

The term “biomarker”, as used in the context of the present invention, represents a characteristic that can be objectively measured and evaluated as an indicator of normal and disease processes or pharmacological responses. A biomarker is a parameter that can be used to measure the onset or the progress of disease or the effects of treatment. The parameter can be chemical, physical or biological.

The term “surrogate biomarker”, as used in the context of the present invention, represents biomarker intended to substitute for a clinical endpoint. It is a measure of a clinical condition or a measure of effect of a certain treatment that may correlate with the real clinical condition (e.g. healthy, diseased) but doesn't necessarily have a guaranteed relationship. An ideal surrogate biomarker is a laboratory substitute for a clinically meaningful result, and should lie directly in the causal pathway linking disease to outcome. Surrogate biomarkers are used when the primary endpoint is undesired (e.g. death). A commonly used example is cholesterol: while elevated cholesterol levels increase the likelihood for heart disease, the relationship is not linear—many people with normal cholesterol develop heart disease, and many with high cholesterol do not. “Death from heart disease” is the endpoint of interest, but “cholesterol” is the surrogate biomarker.

The term “diagnosis” as used in the context of the present invention refers to the process of determining a possible disease or disorder and therefore is a process attempting to define the (clinical) condition of a subject. The determination of the expression level of a set of miRNAs according to the present invention correlates with the (clinical) condition of a subject. Preferably, the diagnosis comprises (i) determining the occurrence/presence of BPH, (ii) monitoring the course of BPH, (iii) staging of BPH, (iv) measuring the response of a patient with BPH to therapeutic intervention, and/or (v) segmentation of a subject suffering from BPH.

The term “prognosis” as used in the context of the present invention refers to describing the likelihood of the outcome or course of a disease or a disorder. Preferably, the prognosis comprises (i) identifying of a subject who has a risk to develop BPH, (ii) predicting/estimating the occurrence, preferably the severity of occurrence of BPH, and/or (iii) predicting the response of a subject with BPH to therapeutic intervention.

The term “(clinical) condition” (biological state or health state), as used herein, means a status of a subject that can be described by physical, mental or social criteria. It includes so-called “healthy” and “diseased” conditions. For the definition of “healthy” and “diseased” conditions it is referred to the international classification of diseases (ICD) of the WHO (http://www.int/classifications/icd/en/index.html). When one condition is compared according to a preferred embodiment of the method of the present invention, it is understood that said condition is BPH or a specific form of BPH. When two or more conditions are compared according to another preferred embodiment of the method of the present invention, it is understood that this is possible for all conditions that can be defined and is not limited to a comparison of a diseased versus healthy comparison and extends to multiway comparison, under the proviso that at least one condition is BPHs, preferably a specific form of BPH.

The term “miRNA expression profile” as used in the context of the present invention, represents the determination of the miRNA expression level or a measure that correlates with the miRNA expression level in a biological sample. The miRNA expression profile may be generated by any convenient means, e.g. nucleic acid hybridization (e.g. to a microarray), nucleic acid amplification (PCR, RT-PCR, qRT-PCR, high-throughput RT-PCR), ELISA for quantitation, next generation sequencing (e.g. ABI SOLID, Illumina Genome Analyzer, Roche/454 GS FLX), flow cytometry (e.g. LUMINEX) and the like, that allow the analysis of differential miRNA expression levels between samples of a subject (e.g. diseased) and a control subject (e.g. healthy, reference sample). The sample material measure by the aforementioned means may be total RNA, labeled total RNA, amplified total RNA, cDNA, labeled cDNA, amplified cDNA, miRNA, labeled miRNA, amplified miRNA or any derivatives that may be generated from the aforementioned RNA/DNA species. By determining the miRNA expression profile, each miRNA is represented by a numerical value. The higher the value of an individual miRNA, the higher is the expression level of said miRNA, or the lower the value of an individual miRNA, the lower is the expression level of said miRNA.

The “miRNA expression profile”, as used herein, represents the expression level/expression data of a single miRNA or a collection of expression levels of at least two miRNAs, preferably of least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more, or up to all known miRNAs.

The term “differential expression” of miRNAs as used herein, means qualitative and/or quantitative differences in the temporal and/or local miRNA expression patterns, e.g. within and/or among biological samples, body fluid samples, cells, or within blood. Thus, a differentially expressed miRNA may qualitatively have its expression altered, including an activation or inactivation in, for example, blood from a diseases subject versus blood from a healthy subject. The difference in miRNA expression may also be quantitative, e.g. in that expression is modulated, i.e. either up-regulated, resulting in an increased amount of miRNA, or down-regulated, resulting in a decreased amount of miRNA. The degree to which miRNA expression differs need only be large enough to be quantified via standard expression characterization techniques, e.g. by quantitative hybridization (e.g. to a microarray), amplification (PCR, RT-PCR, qRT-PCR, high-throughput RT-PCR), ELISA for quantitation, next generation sequencing (e.g. ABI SOLID, Illumina Genome Analyzer, Roche 454 GS FL), flow cytometry (e.g. LUMINEX) and the like.

Nucleic acid hybridization may be performed using a microarray/biochip or in situ hybridization. In situ hybridization is preferred for the analysis of a single miRNA or a set comprising a low number of miRNAs (e.g. a set of at least 2 to 50 miRNAs such as a set of 2, 5, 10, 20, 30, or 40 miRNAs). The microarray/biochip, however, allows the analysis of a single miRNA as well as a complex set of miRNAs (e.g. a all known miRNAs or subsets thereof).

For nucleic acid hybridization, for example, the polynucleotides (probes) according to the present invention with complementarity to the corresponding miRNAs to be detected are attached to a solid phase to generate a microarray/biochip (e.g. 184 polynucleotides (probes) which are complementary to the 184 miRNAs having SEQ ID NO: 1 to 182 and 352-353. Said microarray/biochip is then incubated with a biological sample containing miRNAs, isolated (e.g. extracted) from the body fluid sample such as blood sample from a subject such as a human or an animal, which may be labelled, e.g. fluorescently labelled, or unlabelled. Quantification of the expression level of the miRNAs may then be carried out e.g. by direct read out of a label or by additional manipulations, e.g. by use of a polymerase reaction (e.g. template directed primer extension, MPEA-Assay, RAKE-assay) or a ligation reaction to incorporate or add labels to the captured miRNAs.

Alternatively, the polynucleotides which are at least partially complementary (e.g. a set of chimeric polynucleotides with each a first stretch being complementary to a set of miRNA sequences and a second stretch complementary to capture probes bound to a solid surface (e.g. beads, Luminex beads)) to miRNAs having SEQ ID NO: 1 to 182 and 352-353. are contacted with the biological sample containing miRNAs (e.g a body fluid sample, preferably a blood sample) in solution to hybridize. Afterwards, the hybridized duplexes are pulled down to the surface (e.g a plurality of beads) and successfully captured miRNAs are quantitatively determined (e.g. FlexmiR-assay, FlexmiR v2 detection assays from Luminex).

Nucleic acid amplification may be performed using real time polymerase chain reaction (RT-PCR) such as real time quantitative polymerase chain reaction (RT qPCR). The standard real time polymerase chain reaction (RT-PCR) is preferred for the analysis of a single miRNA or a set comprising a low number of miRNAs (e.g. a set of at least 2 to 50 miRNAs such as a set of 2, 5, 10, 20, 30, or 40 miRNAs), whereas high-throughput RT-PCR technologies (e.g. OpenArray from Applied Biosystems, SmartPCR from Wafergen, Biomark System from Fluidigm) are also able to measure large sets (e.g a set of 10, 20, 30, 50, 80, 100, 200 or more) to all known miRNAs in a high parallel fashion. RT-PCR is particularly suitable for detecting low abandoned miRNAs.

The aforesaid real time polymerase chain reaction (RT-PCR) may include the following steps:

(i) extracting the total RNA from a biological sample or body fluid sample such as a blood sample (e.g. whole blood, serum, or plasma) of a subjects such as human or animal, and obtaining cDNA samples by RNA reverse transcription (RT) reaction using universal or miRNA-specific primers; or collecting a body fluid sample such as urine or blood sample (e.g. whole blood, serum, or plasma) of a patient such as human or animal, and conducting reverse transcriptase reaction using universal or miRNA-specific primers (e.g. looped RT-primers) within the body fluid sample such as urine or blood sample (e.g. whole blood, serum, or plasma) being a buffer so as to prepare directly cDNA samples, (ii) designing miRNA-specific cDNA forward primers and providing universal reverse primers to amplify the cDNA via polymerase chain reaction (PCR), (iii) adding a fluorescent dye (e.g. SYBR Green) or a fluorescent probe (e.g. Taqman probe) probe to conduct PCR, and (iv) detecting the miRNA(s) level in the body fluid sample such as urine or blood sample (e.g. whole blood, serum, or plasma).

A variety of kits and protocols to determine an expression profile by real time polymerase chain reaction (RT-PCR) such as real time quantitative polymerase chain reaction (RT qPCR) are available. For example, reverse transcription of miRNAs may be performed using the TaqMan MicroRNA Reverse Transcription Kit (Applied Biosystems) according to manufacturer's recommendations. Briefly, miRNA may be combined with dNTPs, MultiScribe reverse transcriptase and the primer specific for the target miRNA. The resulting cDNA may be diluted and may be used for PCR reaction. The PCR may be performed according to the manufacturer's recommendation (Applied Biosystems). Briefly, cDNA may be combined with the TaqMan assay specific for the target miRNA and PCR reaction may be performed using ABI7300. Alternative kits are available from Ambion, Roche, Qiagen, Invitrogen, SABiosciences, Exiqon etc.

The term “subject”, as used in the context of the present invention, means a patient or individual or mammal suspected to be affected by BPH. The patient may be diagnosed to be affected by BPH, i.e. diseased, or may be diagnosed to be not affected by BPH, i.e. healthy. The subject may also be diagnosed to be affected by a specific form of BPH. The subject may further be diagnosed to develop BPH or a specific form of BPH as the inventors of the present invention surprisingly found that miRNAs representative for BPH are already present in the biological sample, e.g. blood sample, before BPH occurs or during the early stage of BPH. It should be noted that a subject that is diagnosed as being healthy, i.e. not suffering from BPH or from a specific form of BPH, may possibly suffer from another disease not tested/known. The subject may be any mammal, including both a human and another mammal, e.g. an animal such as a rabbit, mouse, rat, or monkey. Human subjects are particularly preferred. Therefore, the miRNA from a subject may be a human miRNA or a miRNA from another mammal, e.g. an animal miRNA such as a mouse, monkey or rat miRNA, or the miRNAs comprised in a set may be human miRNAs or miRNAs from another mammal, e.g. animal miRNAs such as mouse, monkey or rat miRNAs.

The term “control subject”, as used in the context of the present invention, may refer to a subject known to be affected with BPH (positive control), i.e. diseased, or to a subject known to be not affected with BPH (negative control), i.e. healthy. It may also refer to a subject known to be effected by another disease/condition (see definition “(clinical) condition”). It should be noted that a control subject that is known to be healthy, i.e. not suffering from BPH, may possibly suffer from another disease not tested/known. The control subject may be any mammal, including both a human and another mammal, e.g. an animal such as a rabbit, mouse, rat, or monkey. Human “control subjects” are particularly preferred.

The term “set comprising at least two miRNAs representative for BPH”, as used herein, refers to refers to at least two fixed defined miRNAs comprised in a set which are known to be differential between subjects (e.g. humans or other mammals such as animals) suffering from BPH (diseased state) and control subjects (e.g. humans or other mammals such as animals and are, thus, representative for BPH. Said “set comprising at least two miRNAs representative for BPH” are preferably selected from the group consisting of SEQ ID NO: 1 to 182 and 352-353, a fragment thereof, and a sequence having at least 80% sequence identity thereto.

The term “BPH”, as used herein refers to benign prostatic hyperplasia, which means a benign enlargement of the prostate. BPH can be a progressive disease, especially if left untreated. Though the prostate continues to grow during most of a man's life, the enlargement doesn't usually cause problems until late in life. BPH rarely causes symptoms before age 40, but more than half of men in their sixties and as many as 90 percent in their seventies and eighties have some symptoms of BPH. But more importantly, the symptoms of BPH and prostate cancer are often similar. Often, blood tests are performed to rule out prostatic malignancy. Nevertheless, the testing the prostate specific antigen (PSA) does not meet the performance needs. Therefore, there is a urgent need for diagnostic tests that allow to discriminate between BPH and prostate cancer with high accuracy, sensitivity and specificity.

The inventors of the present invention surprisingly found that miRNAs are significantly dysregulated in body fluid samples such as blood of BPH subjects in comparison to a cohort of healthy controls and incomparison to a cohort of prostate cancer patients and thus, miRNAs are appropriated biomarkers for diagnosing and/or prognosing of BPH in a non-invasive fashion or minimal-invasive fashion. Furthermore, the sets of miRNAs of the present invention lead to high performance in diagnosing and/or prognosing of BPH, thus expose very high specificity, sensitivity and accuracy, both in comparison to healthy controls and in comparison to prostate cancer patients. They succeeded in determining the miRNAs that are differentially regulated in body fluid samples from patients having BPH compared to a cohort of healthy controls and compared to a cohort of prostate cancer patients. Additionally, the inventors of the present invention performed hypothesis tests (e.g. t-test, limma-test) or other measurements (e.g. AUC, mutual information) on the expression level of the found miRNAs, in all controls (healthy subjects or prostate cancer subjects respectively) and subjects suffering from BPH. These tests resulted in a significance value (p-value) for each miRNA. This p-value is a measure for the diagnostic power of each of these single miRNAs to discriminate, for example, between the two clinical conditions: healthy controls (healthy subjects—not suffering from BPH), or diseased, (suffering from BPH) or alternatively between prostate cancer patients (not suffering from BPH) or diseased (suffering from BPH). Since a manifold of tests are carried out, one for each miRNA, the p-values may be too optimistic and, thus, over-estimate the actual discriminatory power. Hence, the p-values are corrected for multiple testing by the Benjamini Hochberg approach.

An overview of the miRNAs that are found to be significantly differentially regulated in biological samples of BPH versus healthy controls and that performed best according to t-test, limma-test or AUC is provided in FIG. 2 or FIG. 7 (healthy controls versus BPH subjects). (Experimental details: SEQ ID NO: sequence identification number, miRNA: identifier of the miRNA according to miRBase, median g1: median intensity obtained from microarray analysis for healthy controls, median g2: median intensity obtained from microarray analysis for individuals with BPH, qmedian: ratio of median g1/median g2, log qmedian: log of qmedian, ttest_rawp: p-value obtained when applying t-test, ttest_adjp: adjusted p-value in order to reduce false discovery rate by Benjamini-Hochberg adjustment, AUC: Area under the curve, limma_rawp: p-value obtained when applying limma-test, limma_adjp: adjusted p-value in order to reduce false discovery rate by Benjamini-Hochberg adjustment.). The miRNAs are sorted in order of their t-test significance as described in more detail in the experimental section (see ttest_adjp=adjusted p-value calculated according to ttest). It should be noted that the lower the ttest_adjp value of a single miRNA, the higher is the diagnostic power of said miRNA for diagnosing and/or prognosing of BPH.

An overview of the miRNAs that are found to be significantly differentially regulated in biological samples of BPH versus prostate cancer patients and that performed best according to t-test, limma-test or AUC is provided FIG. 3 (prostate cancer patients versus BPH subjects) (Experimental details: SEQ ID NO: sequence identification number, miRNA: identifier of the miRNA according to miRBase, median g1: median intensity obtained from microarray analysis for prostate cancer patients, median g2: median intensity obtained from microarray analysis for individuals with BPH, qmedian: ratio of median g1/median g2, log qmedian: log of qmedian, ttest_rawp: p-value obtained when applying t-test, ttest_adjp: adjusted p-value in order to reduce false discovery rate by Benjamini-Hochberg adjustment, AUC: Area under the curve, limma_rawp: p-value obtained when applying limma-test, limma_adjp: adjusted p-value in order to reduce false discovery rate by Benjamini-Hochberg adjustment.). The miRNAs, i.e. miRNAs according to SEQ ID NO: 1 to 10, are sorted in order of their t-test significance as described in more detail in the experimental section (see ttest_adjp=adjusted p-value calculated according to ttest). It should be noted that the lower the ttest_adjp value of a single miRNA, the higher is the diagnostic power of said miRNA for diagnosing and/or prognosing of BPH. Further miRNAs that are found to be significantly differentially regulated in biological samples of BPH versus prostate cancer patients are provided in FIG. 8.

Usually the diagnostic power of a single miRNA biomarker is not sufficient to reach high accuracy, specificity and sensitivity for discrimination between healthy subjects or prostate cancer subjects (controls) and subjects suffering from BPH, hence no simple threshold method can be used for diagnosis and/or prognosis.

Therefore, the inventors of the present invention employed more than one miRNA biomarker, i.e. sets of miRNA biomarkers (signatures), to further increase and/or improve the performance for diagnosing and/or prognosing of subjects suffering from BPH. This leads to a significant increase in sensitivity, specificity and accuracy when compared to the prior art.

In order to be able to discriminate, for example, between two or more clinical conditions, e.g. healthy and suffering from BPH, for a defined set of miRNA biomarkers, the inventors of the present invention applied a machine learning approach (e.g. t-test, AUC, support vector machine, hierarchical clustering, or k-means) which leads to an algorithm that is trained by reference data (i.e. data of reference miRNA expression profiles from the two clinical conditions, e.g. healthy and suffering from BPH, for the defined set of miRNA markers) to discriminate between the two statistical classes (i.e. two clinical conditions, e.g. healthy or suffering from BPH).

The inventors of the present invention surprisingly found that this approach yields in miRNA sets (signatures) that provide high diagnostic accuracy, specificity and sensitivity when comparing BPH patients and healthy controls (see miRNA sets SNB 1-968 in FIG. 4 and FIG. 10).

Further, the inventors of the present invention surprisingly found that this approach yields in miRNA sets (signatures) that provide high diagnostic accuracy, specificity and sensitivity when comparing BPH patients and prostate cancer patients (see miRNA sets SPB 1-356 in FIG. 5 or FIG. 11).

An exemplarily approach to arrive at miRNA sets/signatures that correlate with BPH is summarized below:
Step 1: Total RNA (or subfractions thereof) is extracted from the biological sample, e.g. a body fluid sample, preferably a blood sample (including plasma, serum, PBMC or other blood fractions), using suitable kits and/or purification methods.
Step 2: From the respective samples the quantity (expression level) of one miRNA or sets of at least two miRNAs, e.g. selected from the group listed in FIG. 2 or FIG. 7(BPH versus healthy control) or e.g. FIG. 3 or FIG. 8 (BPH versus prostate cancer), is measured using experimental techniques. These techniques include but are not restricted to array based approaches, amplification methods (PCR, RT-PCR, qPCR), sequencing, next generation sequencing, flow cytometry and/or mass spectroscopy.
Step 3: In order to gather information on the diagnostic/prognostic value and the redundancy of each of the single miRNA biomarkers, mathematical methods are applied. These methods include, but are not restricted to, basic mathematic approaches (e.g. Fold Quotients, Signal to Noise ratios, Correlation), statistical methods as hypothesis tests (e.g. t-test, Wilcoxon-Mann-Whitney test), the Area under the Receiver operator Characteristics Curve, information theory approaches, (e.g. the Mutual Information, Cross-entropy), probability theory (e.g. joint and conditional probabilities) or combinations and modifications of the previously mentioned methods.
Step 4: The information gathered in step 3) is used to estimate for each miRNA biomarker the diagnostic content or value. Usually, however, this diagnostic value is too small to get a highly accurate diagnosis with accuracy rates, specificities and sensitivities beyond the 90% barrier.
   The diagnostic content of the miRNAs suitable for diagnosing/prognosing BPH is exemplarily listed in FIG. 2 or 7 or FIG. 3 or 8
Step 5: In order to increase the performance for diagnosing/prognosing of subjects suffering from BPH, more than one miRNA biomarker needs to be employed. Thus statistical learning/machine learning/bioinformatics/computational approaches are applied for set selection in order to select/define sets of miRNA biomarkers that are tailored for the detection of BPH. These techniques include, but are not restricted to, Wrapper subset selection techniques (e.g. forward step-wise, backward step-wise, combinatorial approaches, optimization approaches), filter subset selection methods (e.g. the methods mentioned in Step 3), principal component analysis, or combinations and modifications of such methods (e.g. hybrid approaches).
Step 6: The subsets, selected/defined in Step 5, which may range from only a small number (at least two for the set) to all measured biomarkers is then used to carry out a diagnosis/prognosis of BPH. To this end, statistical learning/machine learning/bioinformatics/computational approaches are applied that include but are not restricted to any type of supervised or unsupervised analysis: classification techniques (e.g. naïve Bayes, Linear Discriminant Analysis, Quadratic Discriminant Analysis Neural Nets, Tree based approaches, Support Vector Machines, Nearest Neighbour Approaches), Regression techniques (e.g. linear Regression, Multiple Regression, logistic regression, probit regression, ordinal logistic regression ordinal Probit-Regression, Poisson Regression, negative binomial Regression, multinomial logistic Regression, truncated regression), Clustering techniques (e.g. k-means clustering, hierarchical clustering, PCA), Adaptations, extensions, and combinations of the previously mentioned approaches.
Step 7: By combination of subset selection (Step 5) and machine learning (Step 6) an algorithm or mathematical function for diagnosing/prognosing BPH is obtained. This algorithm or mathematical function is applied to a miRNA expression profile of a subject to be diagnosed for BPH.

This approach results in sets of miRNAs that are suitable for diagnosing/prognosing BPH, preferably when comparing healthy controls and BPH patients (see FIG. 4 or 10 for suitable miRNA sets) or alternatively when comparing prostate cancer patients and BPH patients (see FIG. 5 or 11 for suitable miRNA sets)

In a first aspect, the present invention relates to a method for diagnosing and/or prognosing of BPH comprising the steps of:
(i) determining an expression profile of a set comprising at least two miRNAs representative for BPH in a body fluid sample from a subject, and
(ii) comparing said expression profile to a reference expression profile, wherein the comparison of said expression profile to said reference expression profile allows for the diagnosis and/or prognosis of BPH,

It is preferred that the body fluid sample is a blood sample, preferably a blood cell sample or a leukocyte containing sample, particularly preferred it is a whole blood, PBMC, serum or plasma sample, more particularly preferred it is a whole blood sample.

It is preferred that the subject is a mammal including both a human and another mammal, e.g. an animal such as a mouse, a rat, a rabbit, or a monkey. It is particularly preferred that the subject is a human.

In one embodiment of the first aspect of the present invention, the set of miRNAs comprises miRNAs that are differentially regulated in BPH patients as compared to healthy controls.

Preferably, the set comprising at least two miRNAs that are differentially regulated in BPH patients as compared to healthy controls is selected from the set of miRNAs listed in FIG. 2 or FIG. 7.

It is preferred that the set comprising at least two miRNAs that are differentially regulated in BPH patients as compared to healthy controls is selected from the sets of miRNAs listed in FIG. 4 or FIG. 10 (SNB-1 to SNB-968).

It is also preferred that the set comprising at least two miRNAs that are differentially regulated in BPH patients as compared to healthy controls comprises at least one set of miRNAs listed in FIG. 4 or FIG. 10.

Further, according to the method of the present invention, for determining an expression profile of the set comprising at least two miRNAs that are differentially regulated in BPH patients as compared to healthy controls comprises the miRNAs from one set or a plurality of sets of miRNAs listed in FIG. 4 or FIG. 10.

For example, a set comprising 30 miRNAs that are differentially regulated in BPH patients as compared to healthy controls in a body fluid sample from a subject comprises at least the miRNAs from one set or several sets of miRNAs listed in FIG. 4. Alternatively, a set comprising 29, 28, 27, 26, 25, 24, 23, 22, 21, 20, 19, 18, 17, 16, 15, 14, 13, 12, 11, 10, 9, 8, 7, 6, 5, 4 or 3 miRNAs that are differentially regulated in BPH patients as compared to healthy controls comprises at least the miRNAs from one set or several sets of miRNAs listed in FIG. 4 or FIG. 10.

Further, according to the method of the present invention, for determining an expression profile of the set comprising at least two that are differentially regulated in BPH patients as compared to healthy controls in a body fluid sample from a subject comprises combinations of sets of miRNAs listed in FIG. 4 or FIG. 10. For example, said set comprising 30 miRNAs that are differentially regulated in BPH patients as compared to healthy controls in a body fluid sample from a subject comprises at least 2, e.g. 2, 3, 4, 5 or 6, sets of miRNAs listed in FIG. 4 or FIG. 10. Alternatively, said set comprising 29, 28, 27, 26, 25, 24, 23, 22, 21, 20, 19, 18, 17, 16, 15, 14, 13, 12, 11, 10, 9, 8, 7, 6, 5 or 4 miRNAs comprises a least 2, e.g. 2, 3, 4, 5 or 6, sets of miRNAs listed in FIG. 4 or FIG. 10.

The reference expression profile may be obtained from at least two subjects (e.g. human or animal). Preferably, the reference expression profile is an average expression profile (data) of at least 2 to 400 subjects, more preferably at least 20 to 200 subjects, and most preferably at least 40 to 150 subjects, with two known clinical conditions which are BPH or a specific form of BPH and healthy control.

In second embodiment of the first aspect of the present invention, the set of miRNAs comprises miRNAs that are differentially regulated in BPH patients as compared to prostate cancer patients.

Preferably, the set comprising at least two miRNAs that are differentially regulated in BPH patients as compared to prostate cancer patients is selected from the set of miRNAs listed in FIG. 3 or FIG. 8.

It is preferred that the set comprising at least two miRNAs that are differentially regulated in BPH patients as compared to prostate cancer patients is selected from the sets of miRNAs listed in FIG. 5 (SPB-1 to SPB-247) or FIG. 11 (SPB-248-356).

It is also preferred that the set comprising at least two miRNAs that are differentially regulated in BPH patients as compared to prostate cancer patients comprises at least one set of miRNAs listed in FIG. 5 or FIG. 11.

Further, according to the method of the present invention, for determining an expression profile of the set comprising at least two miRNAs that are differentially regulated in BPH patients as compared to prostate cancer patients in a body fluid sample from a subject comprises the miRNAs from one set or a plurality of sets of miRNAs listed in FIG. 5 or FIG. 11.

For example, a set comprising 30 miRNAs that are differentially regulated BPH patients as compared to prostate cancer patients in a body fluid sample from a subject comprises at least the miRNAs from one set or several sets of miRNAs listed in FIG. 5 or FIG. 11. Alternatively, a set comprising 29, 28, 27, 26, 25, 24, 23, 22, 21, 20, 19, 18, 17, 16, 15, 14, 13, 12, 11, 10, 9, 8, 7, 6, 5, 4 or 3 miRNAs that are differentially regulated in BPH patients as compared to prostate cancer patients comprises at least the miRNAs from one set or several sets of miRNAs listed in FIG. 5 or FIG. 11.

Further, according to the method of the present invention, for determining an expression profile of the set comprising at least two that are differentially regulated in BPH patients as compared to prostate cancer patients in a body fluid sample from a subject comprises combinations of sets of miRNAs listed in FIG. 5 or FIG. 11. For example, said set comprising 30 miRNAs that are differentially regulated in BPH patients as compared to prostate cancer patients in a body fluid sample from a subject comprises at least 2, e.g. 2, 3, 4, 5 or 6, sets of miRNAs listed in FIG. 5 or FIG. 11. Alternatively, said set comprising 29, 28, 27, 26, 25, 24, 23, 22, 21, 20, 19, 18, 17, 16, 15, 14, 13, 12, 11, 10, 9, 8, 7, 6, 5 or 4 miRNAs comprises a least 2, e.g. 2, 3, 4, 5 or 6, sets of miRNAs listed in FIG. 5 or FIG. 11.

The reference may be a reference expression profile that may be obtained from at least two subjects (e.g. human or animal). Preferably the reference expression profile is an average expression profile (data) of at least 2 to 200 subjects, more preferably at least 10 to 150 subjects, and most preferably at least 20 to 100 subjects, with two known clinical conditions which are BPH or a specific form of BPH and prostate cancer or a specific form of prostate cancer.

It is particularly preferred that the reference is an algorithm or mathematical function. Preferably the algorithm or mathematical function is obtained from a reference expression profile (data) of at least two subjects, preferably the algorithm or mathematical function is obtained from an average reference expression profile (data) of at least 2 to 200 subjects, more preferably of at least 10 to 150 subjects, and most preferably of at least 20 to 100 subjects.

It is preferred that the algorithm or mathematical function is obtained using a machine learning approach

Preferably, the algorithm or mathematical function is saved on a data carrier comprised in the kit (according to the seventh aspect of the invention) or the computer program, wherein the algorithm or mathematical function is comprised, is saved on a data carrier comprised in the kit.

Preferably, the machine learning approach involves the following steps:
(i) inputting the reference expression profile(s) of (a) subject(s) with the known clinical condition of BPH or condition and/or with any other known clinical condition(s), preferably with the known clinical condition of healthy control or alternatively prostate cancer, and
(ii) computing an algorithm or a mathematical function based on said reference expression profile(s) that is suitable to distinguish between the (likely) clinical condition of BPH or any other (likely) clinical condition(s), preferably the clinical condition of healthy control or alternatively prostate cancer, or to decide if the clinical condition of BPH or any other condition, or no periodontal disease or condition is present or will likely be present in said patient.

It is preferred that the miRNA expression profile may be generated by any convenient means, e.g. nucleic acid hybridization (e.g. to a microarray), nucleic acid amplification (PCR, RT-PCR, qRT-PCR, high-throughput RT-PCR), ELISA for quantitation, next generation sequencing (e.g. ABI SOLID, Illumina Genome Analyzer, Roche/454 GS FLX), flow cytometry (e.g. LUMINEX) and the like, that allow the analysis of differential miRNA expression levels between samples of a subject (e.g. diseased) and a control subject (e.g. healthy, reference sample).

Nucleic acid hybridization may be performed using a microarray/biochip or in situ hybridization. In situ hybridization is preferred for the analysis of a single miRNA or a set comprising a low number of miRNAs (e.g. a set of at least 2 to 50 miRNAs such as a set of 2, 5, 10, 20, 30, or 40 miRNAs). The microarray/biochip, however, allows the analysis of a single miRNA as well as a complex set of miRNAs (e.g. a all known miRNAs or subsets thereof).

Nucleic acid amplification may be performed using real time polymerase chain reaction (RT-PCR) such as real time quantitative polymerase chain reaction (RT qPCR). The standard real time polymerase chain reaction (RT-PCR) is preferred for the analysis of a single miRNA or a set comprising a low number of miRNAs (e.g. a set of at least 2 to 50 miRNAs such as a set of 2, 5, 10, 20, 30, or 40 miRNAs), whereas high-throughput RT-PCR technologies (e.g. OpenArray from Applied Biosystems, SmartPCR from Wafergen, Biomark System from Fluidigm) are also able to measure large sets of miRNAS (e.g a set of 10, 20, 30, 50, 80, 100, 200 or more) or all known miRNAs in a high parallel fashion. RT-PCR is particularly suitable for detecting low abandoned miRNAs.

In a second aspect, the invention relates to a set comprising polynucleotides for detecting a set comprising at least two miRNAs for diagnosing and/or prognosing of BPH in a body fluid sample from a subject.

It is preferred that the body fluid sample is a blood sample, preferably a blood cell sample or a leukocyte containing sample, particularly preferred it is a whole blood, PBMC, serum or plasma sample, more particularly preferred it is a whole blood sample.

It is preferred that the subject is a mammal including both a human and another mammal, e.g. an animal such as a mouse, a rat, a rabbit, or a monkey. It is particularly preferred that the subject is a human.

In one embodiment of the second aspect of the present invention, the set of miRNAs comprises miRNAs that are differentially regulated in BPH patients as compared to healthy controls.

Preferably, the set comprising at least two miRNAs that are differentially regulated in BPH patients as compared to healthy controls is selected from the set of miRNAs listed in FIG. 2 or FIG. 7.

It is preferred that the set comprising at least two miRNAs that are differentially regulated in BPH patients as compared to healthy controls is selected from the set of miRNAs listed in FIG. 4 or FIG. 10 (SNB 1-968).

It is preferred that the set comprising at least two miRNAs that are differentially regulated in BPH patients as compared to healthy controls. comprises at least one set of miRNAs listed in FIG. 4 or FIG. 10.

It is preferred that the polynucleotides comprised in the set of the present invention are complementary to the miRNAs comprised in the set, wherein the nucleotide sequences of said miRNAs are preferably selected from the group consisting of miRNAs listed in FIG. 2 or FIG. 7 or set of miRNAs listed in FIG. 4 or FIG. 10, a fragment thereof, and a sequence having at least 80%, 85%, 90% or 95% sequence identity thereto.

For example, the polynucleotides of the present invention are for detecting a set of 40 or 39 or 38 or 37 or 36 or 35 or 34 or 33 or 32 or 31 or 30 or 29 or 28 or 27 or 26 or 25 or 24 or 23 or 22 or 21 or 20 or 19 or 18 or 17 or 16 or 15 or 14 or 13 or 12 or 11 or 10 or 9 or 8 or 7 or 6 or 5 or 4 or 3 miRNAs that are differentially regulated in BPH patients as compared to healthy controls wherein the set of miRNAs comprises at least one, e.g. 1, 2, 3, 4, 5 or 6, of the set of miRNAs listed in FIG. 4 or FIG. 10.

In a second embodiment of the second aspect of the present invention, the set of miRNAs comprises miRNAs that are differentially regulated in BPH patients as compared to prostate cancer patients.

Preferably, the set comprising at least two miRNAs that are differentially regulated in BPH patients as compared to prostate cancer patients is selected from the set of miRNAs listed in FIG. 3 or FIG. 8.

It is preferred that the set comprising at least two miRNAs that are differentially regulated in BPH patients as compared to prostate cancer patients is selected from the set of miRNAs listed in FIG. 5 or FIG. 11.

It is preferred that the set comprising at least two miRNAs that are differentially regulated in BPH patients as compared to prostate cancer patients. comprises at least one set of miRNAs listed in FIG. 5 or FIG. 11.

It is preferred that the polynucleotides comprised in the set of the present invention are complementary to the miRNAs comprised in the set, wherein the nucleotide sequences of said miRNAs are preferably selected from the group consisting of miRNAs listed in FIG. 3 or FIG. 8 or set of miRNAs listed in FIG. 5 or FIG. 11, a fragment thereof, and a sequence having at least 80%, 85%, 90% or 95% sequence identity thereto.

For example, the polynucleotides of the present invention are for detecting a set of 40 or 39 or 38 or 37 or 36 or 35 or 34 or 33 or 32 or 31 or 30 or 29 or 28 or 27 or 26 or 25 or 24 or 23 or 22 or 21 or 20 or 19 or 18 or 17 or 16 or 15 or 14 or 13 or 12 or 11 or 10 or 9 or 8 or 7 or 6 or 5 or 4 or 3 miRNAs that are differentially regulated in BPH patients as compared to prostate cancer patients wherein the set of miRNAs comprises at least one, e.g. 1, 2, 3, 4, 5 or 6, of the set of miRNAs listed in FIG. 5 or FIG. 11.

In a third aspect, the invention relates to the use of set of polynucleotides according to the second aspect of the invention for diagnosing and/or prognosing BPH in a subject.

In a fourth aspect, the invention relates to a set of at least two primer pairs for determining the expression level of a set of miRNAs in a body fluid sample of a subject suffering or suspected of suffering from BPH.

It is preferred that the body fluid sample is a blood sample, preferably a blood cell sample or a leukocyte containing sample, particularly preferred it is a whole blood, PBMC, serum or plasma sample, more particularly preferred it is a whole blood sample.

It is preferred that the subject is a mammal including both a human and another mammal, e.g. an animal such as a mouse, a rat, a rabbit, or a monkey. It is particularly preferred that the subject is a human.

In one embodiment of the third aspect of the present invention, the set of miRNAs comprises miRNAs that are differentially regulated in BPH patients as compared to healthy controls.

Preferably, the set comprising at least two miRNAs that are differentially regulated in BPH patients as compared to healthy controls is selected from the set of miRNAs listed in FIG. 2 or FIG. 7.

It is preferred that the set comprising at least two miRNAs that are differentially regulated in BPH patients as compared to healthy controls is selected from the sets of miRNAs listed in FIG. 4 or FIG. 10.

It is preferred that the set comprising at least two miRNAs that are differentially regulated in BPH patients as compared to healthy controls comprises at least one set of miRNAs listed in FIG. 4 or FIG. 10.

It is preferred that the set of at least two primer pairs for determining the expression level of a set of miRNAs that are differentially regulated in BPH patients as compared to healthy controls are primer pairs that are specific for at least one miRNA listed in FIG. 2 or FIG. 7.

It is preferred that the set of at least two primer pairs for determining the expression level of a set of miRNAs that are differentially regulated in BPH patients as compared to healthy controls are primer pairs that are specific for at least one set of miRNAs listed in FIG. 4 or FIG. 10.

It is preferred that the set of at least two primer pairs of the present invention are for detecting a set comprising, essentially consisting of, or consisting of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40 or more miRNAs, and wherein the set of miRNAs comprises at least one of the sets listed in FIG. 4 or FIG. 10.

For example, the set of at least two primer pairs of the present invention are for detecting a set of 40 or 39 or 38 or 37 or 36 or 35 or 34 or 33 or 32 or 31 or 30 or 29 or 28 or 27 or 26 or 25 or 24 or 23 or 22 or 21 or 20 or 19 or 18 or 17 or 16 or 15 or 14 or 13 or 12 or 11 or 10 or 9 or 8 or 7 or 6 or 5 or 4 or 3 or 2 miRNAs wherein the set of miRNAs that are differentially regulated in BPH patients as compared to healthy controls comprises at least one of the set of miRNAs listed in FIG. 4 or FIG. 10.

Preferably, the said primer pairs may be used for amplifying cDNA transcripts of the set of miRNAs that are differentially regulated in BPH patients as compared to healthy controls selected from the miRNAs listed in FIG. 2 or FIG. 7. Furthermore, the said primer pairs may be used for amplifying cDNA transcripts of the set of miRNAs listed in FIG. 4 or FIG. 10.

It is understood that the primer pairs for detecting a set of miRNAs may consist of specific and or non-specific primers. Additionally, the set of primer pairs may be complemented by other substances or reagents (e.g. buffers, enzymes, dye, labelled probes) known to the skilled in the art for conducting real time polymerase chain reaction (RT-PCR).

In a second embodiment of the third aspect of the present invention, the set of miRNAs comprises miRNAs that are differentially regulated in BPH patients as compared to prostate cancer patients.

Preferably, the set comprising at least two miRNAs that are differentially regulated in BPH patients as compared to prostate cancer patients is selected from the set of miRNAs listed in FIG. 3 or FIG. 8.

It is preferred that the set comprising at least two miRNAs that are differentially regulated in BPH patients as compared to prostate cancer patients is selected from the sets of miRNAs listed in FIG. 5 or FIG. 11.

It is preferred that the set comprising at least two miRNAs that are differentially regulated in BPH patients as compared to prostate cancer patients comprises at least one set of miRNAs listed in FIG. 5 or FIG. 11.

It is preferred that the set of at least two primer pairs for determining the expression level of a set of miRNAs that are differentially regulated in BPH patients as compared to prostate cancer patients are primer pairs that are specific for at least one miRNA listed in FIG. 3 or FIG. 8.

It is preferred that the set of at least two primer pairs for determining the expression level of a set of miRNAs that are differentially regulated in BPH patients as compared to prostate cancer patients are primer pairs that are specific for at least one set of miRNAs listed in FIG. 5 or FIG. 11.

It is preferred that the set of at least two primer pairs of the present invention are for detecting a set comprising, essentially consisting of, or consisting of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40 or more miRNAs, and wherein the set of miRNAs comprises at least one of the sets listed in FIG. 5 or FIG. 11.

For example, the set of at least two primer pairs of the present invention are for detecting a set of 40 or 39 or 38 or 37 or 36 or 35 or 34 or 33 or 32 or 31 or 30 or 29 or 28 or 27 or 26 or 25 or 24 or 23 or 22 or 21 or 20 or 19 or 18 or 17 or 16 or 15 or 14 or 13 or 12 or 11 or 10 or 9 or 8 or 7 or 6 or 5 or 4 or 3 or 2 miRNAs wherein the set of miRNAs that are differentially regulated in BPH patients as compared to prostate cancer patients comprises at least one of the set of miRNAs listed in FIG. 5 or FIG. 11.

Preferably, the said primer pairs may be used for amplifying cDNA transcripts of the set of miRNAs that are differentially regulated in BPH patients as compared to prostate cancer patients selected from the miRNAs listed in FIG. 3 or FIG. 8. Furthermore, the said primer pairs may be used for amplifying cDNA transcripts of the set of miRNAs listed in FIG. 5 or FIG. 11.

It is understood that the primer pairs for detecting a set of miRNAs may consist of specific and or non-specific primers. Additionally, the set of primer pairs may be complemented by other substances or reagents (e.g. buffers, enzymes, dye, labelled probes) known to the skilled in the art for conducting real time polymerase chain reaction (RT-PCR).

In a fifth aspect, the invention relates to the use of a set of primer pairs according to the fourth aspect of the invention for diagnosing and/or prognosing BPH in a subject.

In a sixth aspect, the invention relates to means for diagnosing and/or prognosing of BPH in a body fluid sample of a subject.

Preferably, the invention relates to means for diagnosing and/or prognosing of BPH in a body fluid sample of a subject comprising
(i) a set of at least two polynucleotides according to the second aspect of the invention or
(ii) a set of at least two primer pairs according the fourth aspect of the invention.

It is preferred that the body fluid sample is a blood sample, preferably a blood cell sample or a leukocyte containing sample, particularly preferred it is a whole blood, PBMC, serum or plasma sample, more particularly preferred it is a whole blood sample.

It is preferred that the subject is a mammal including both a human and another mammal, e.g. an animal such as a mouse, a rat, a rabbit, or a monkey. It is particularly preferred that the subject is a human.

In a first embodiment of the sixth aspect of the present invention, the set of at least two polynucleotides or the set of at least 2 primer pairs are for detecting a set comprising at least two miRNAs that are differentially regulated in BPH patients as compared to healthy controls.in a body fluid sample, e.g. blood sample, from a subject, e.g. patient, human or animal, wherein the set of miRNAs is selected from the miRNAs listed in FIG. 2 or FIG. 7.

It is preferred that the set of at least two polynucleotides or the set of at least 2 primer pairs are for detecting a set comprising at least two miRNAs that are differentially regulated in BPH patients as compared to healthy controls.in a body fluid sample, e.g. blood sample, from a subject, e.g. patient, human or animal, wherein the set of miRNAs is selected from the sets of miRNAs listed in FIG. 4 or FIG. 10.

It is preferred that the set of at least two primer pairs for determining the expression level of a set of miRNAs that are differentially regulated in BPH patients as compared to healthy controls. are primer pairs that are specific for at least two miRNAs selected from the miRNAs listed in FIG. 2 or FIG. 7.

It is preferred that the set of at least two primer pairs for determining the expression level of a set of miRNAs that are differentially regulated in BPH patients as compared to healthy controls. are primer pairs that are specific for at least one set of miRNAs listed in FIG. 4 or FIG. 10.

In a second embodiment of the sixth aspect of the present invention, that the set of at least two polynucleotides or the set of at least 2 primer pairs are for detecting a set comprising at least two miRNAs that are differentially regulated in BPH patients as compared to prostate cancer patients in a body fluid sample, e.g. blood sample, from a subject, e.g. patient, human or animal, wherein the set of miRNAs is selected from the miRNAs listed in FIG. 3 or FIG. 8.

It is preferred that the set of at least two polynucleotides or the set of at least 2 primer pairs are for detecting a set comprising at least two miRNAs that are differentially regulated in BPH patients as compared to prostate cancer patients.in a body fluid sample, e.g. blood sample, from a subject, e.g. patient, human or animal, wherein the set of miRNAs is selected from the sets of miRNAs listed in FIG. 5 or FIG. 11.

It is preferred that the set of at least two primer pairs for determining the expression level of a set of miRNAs that are differentially regulated in BPH patients as compared to prostate cancer patients. are primer pairs that are specific for at least two miRNAs selected from the miRNAs listed in FIG. 3 or FIG. 8.

It is preferred that the set of at least two primer pairs for determining the expression level of a set of miRNAs that are differentially regulated in BPH patients as compared to prostate cancer patients. are primer pairs that are specific for at least one set of miRNAs listed in FIG. 5 or FIG. 11.

It is also preferred that said means for diagnosing and/or prognosing of BPH comprise, of a set of beads comprising a at least two polynucleotides according to the second aspect of the present invention. It is especially preferred that the beads are employed within a flow cytometer setup for diagnosing and/or prognosing of BPH, e.g. in a LUMINEX system (www.luminexcorp.com).

In a seventh aspect, the invention relates to a kit for diagnosing and/or prognosing of BPH in a subject.

Preferably, the invention relates to a kit for diagnosing and/or prognosing of BPH comprising
(i) means for determining an expression profile of a set comprising at least two miRNAs representative for BPH in a body fluid sample from a subject, and
(ii) at least one reference.

It is preferred that the body fluid sample is a blood sample, preferably a blood cell sample or a leukocyte containing sample, particularly preferred it is a whole blood, PBMC, serum or plasma sample, more particularly preferred it is a whole blood sample.

It is preferred that the subject is a mammal including both a human and another mammal, e.g. an animal such as a mouse, a rat, a rabbit, or a monkey. It is particularly preferred that the subject is a human.

In a first embodiment of the seventh aspect of the present invention, the set of miRNAs comprises miRNAs that are differentially regulated in BPH patients as compared to healthy controls.

In a second embodiment of the seventh aspect of the present invention, the set of miRNAs comprises miRNAs that are differentially regulated in BPH patients as compared to prostate cancer patients.

Said means may comprise of at least two polynucleotides according to the second aspect of the present invention, a set of at least 2 primer pairs according to the fourth aspect of the invention; means according to the sixth aspect of the present invention; primers suitable to perform reverse transcriptase reaction and/or real time polymerase chain reaction such as quantitative polymerase chain reaction; and/or means for conducting next generation sequencing.

In an eighth aspect, the invention relates to a set of miRNAs in a body fluid sample isolated from a subject for diagnosing and/or prognosing of BPH.

It is preferred that the body fluid sample is a blood sample, preferably a blood cell sample or a leukocyte containing sample, particularly preferred it is a whole blood, PBMC, serum or plasma sample, more particularly preferred it is a whole blood sample.

It is preferred that the subject is a mammal including both a human and another mammal, e.g. an animal such as a mouse, a rat, a rabbit, or a monkey. It is particularly preferred that the subject is a human.

In a first embodiment of the eighth aspect of the present invention, the set of miRNAs comprises miRNAs that are differentially regulated in BPH patients as compared to healthy controls.

Preferably, the set comprising at least two miRNAs that are differentially regulated in BPH patients as compared to healthy controls is selected from the set of miRNAs listed in FIG. 2 or FIG. 7.

It is preferred that the set comprising at least two miRNAs that are differentially regulated in BPH patients as compared to healthy controls is selected from the set of miRNAs listed in FIG. 4 or FIG. 10.

It is preferred that the set comprising at least two miRNAs that are differentially regulated in BPH patients as compared to healthy controls comprises at least one set of miRNAs listed in FIG. 4 or FIG. 10.

In a second embodiment of the eighth aspect of the present invention, the set of miRNAs comprises miRNAs that are differentially regulated in BPH patients as compared to prostate cancer patients.

Preferably, the set comprising at least two miRNAs that are differentially regulated in BPH patients as compared to prostate cancer patients is selected from the set of miRNAs listed in FIG. 3 or FIG. 8.

It is preferred that the set comprising at least two miRNAs that are differentially regulated in BPH patients as compared to prostate cancer patients is selected from the set of miRNAs listed in FIG. 5 or FIG. 11.

It is preferred that the set comprising at least two miRNAs that are differentially regulated in BPH patients as compared to prostate cancer patients comprises at least one set of miRNAs listed in FIG. 5 or FIG. 11.

In a ninth aspect, the invention relates to the use of a set of miRNAs according to the eighth aspect of the invention for diagnosing and/or prognosing of BPH in a subject.

In a tenth aspect, the invention relates to diagnosing and/or prognosing of prostate cancer in a in a body fluid sample, preferably in a blood sample, from a subject Surprisingly, the inventors found out that miRNAs are differentially regulated in samples from prostate cancer patients as compared to health controls. A complete overview of all miRNAs that are found to be differentially is provided in the table shown in FIG. 6 or FIG. 9. In FIG. 6 in total, 279 miRNAs were found to be significantly deregulated (t-test significance <0.05) in blood cells of prostate cancer patients as compared to the healthy controls.

A preferred embodiment of the tenth aspect of the invention relates to a method diagnosing and/or prognosing of prostate cancer comprising the steps of:
(i) determining an expression profile of a set comprising at least two miRNAs representative for prostate cancer in a body fluid sample from a subject, and
(ii) comparing said expression profile to a reference, wherein the comparison of said expression profile to said reference allows for the diagnosis and/or prognosis of prostate cancer,
   wherein the set of miRNAs comprises miRNAs that are differentially regulated in blood samples of prostate cancer subjects as compared to healthy controls and wherein the miRNAs are selected from the tables in FIG. 6 or FIG. 9.

It is preferred that the body fluid sample according to the tenth aspect of the invention is a blood sample, preferably a blood cell sample or a leukocyte containing sample, particularly preferred it is a whole blood, PBMC, serum or plasma sample, more particularly preferred it is a whole blood sample.

Another preferred embodiment of the tenth aspect of the invention relates to a set comprising polynucleotides for detecting a set comprising at least two miRNAs for diagnosing and/or prognosing of prostate cancer in a body fluid sample from a subject, wherein the set of miRNAs comprises at least one miRNA listed in FIG. 6 or FIG. 9 or a set or a combination of sets of miRNAs listed in FIG. 12.

Another preferred embodiment of the tenth aspect of the invention relates to the use of the aforementioned set of polynucleotides for diagnosing and/or prognosing prostate cancer in a subject.

Another preferred embodiment of the tenth aspect of the invention relates to a set of primer pairs for determining the expression level of a set of miRNAs in a body fluid sample of a subject suffering or suspected of suffering from prostate cancer, wherein the set of miRNAs comprises at least one miRNA listed in FIG. 6 or FIG. 9.

Another preferred embodiment of the tenth aspect of the invention relates to the use of set of the aforementioned primer pairs for diagnosing and/or prognosing prostate cancer in a subject.

Another preferred embodiment of the tenth aspect of the invention provides means for diagnosing and/or prognosing of prostate cancer in a body fluid sample of a subject comprising:
(i) a set of at least two polynucleotides, or
(ii) a set of primer pairs.

Another preferred embodiment of the tenth aspect of the invention provides a kit for diagnosing and/or prognosing of prostate cancer comprising
(i) means for determining an expression profile of a set comprising at least two miRNAs representative for prostate cancer in a body fluid sample from a subject, and
(ii) at least one reference.

Another preferred embodiment of the tenth aspect of the invention provides a set of miRNAs in a body fluid sample isolated from a subject for diagnosing and/or prognosing of prostate cancer, wherein the set of miRNAs comprises at least one miRNA listed in FIG. 6 or FIG. 9 or a set or a combination of sets of miRNAs listed in FIG. 12.

Another preferred embodiment of the tenth aspect of the invention provides a use of the aforementioned set of miRNAs for diagnosing and/or prognosing of prostate cancer in a subject,

In summary, the present invention is composed of the following items:
1. A method of diagnosing BPH, comprising the steps
   (a) determining an expression profile of a set of miRNAs, in a blood sample from a patient; and
   (b) comparing said expression profile to a reference,
   wherein the comparison of said determined expression profile to said reference expression profile allows for the diagnosis of BPH.
2. The method according to item 1, wherein the blood sample is a blood cell sample or a leukocyte containing sample.
3. The method according to item 1 or 2, wherein the set of miRNAs comprises miRNAs that are differentially regulated in blood samples from BPH patients as compared to prostate cancer patients
4. The method according to any of the items 1, 2 or 3, wherein the expression profile is determined from miRNAs selected from FIG. 3 or FIG. 8
5. The method according to any of the items 1, 2, 3 or 4, wherein the set of miRNAs comprises at least one set of miRNAs listed in FIG. 5 or FIG. 11
6. The method according to item 1 or 2, wherein the set of miRNAs comprises miRNAs that are differentially regulated in blood samples from BPH patients as compared to healthy controls
7. The method according to any of the items 1, 2 or 6, wherein the expression profile is determined from miRNAs selected from FIG. 2 or FIG. 7
8. The method according to any of the items 1, 2, 6 or 7, wherein the set of miRNAs comprises at least one set of miRNAs listed in FIG. 4 or FIG. 10
9. The method according to any of the items 1 to 8, wherein the set of miRNAs representative for diagnosis of BPH comprises at least 1, 7, 10, 15, 20, 25, 30, 35, 40, 50, 75, 100 of miRNAs.
10. The method according to any one of items 1 to 9 wherein the expression profile is determined by nucleic acid hybridization, nucleic acid amplification, polymerase extension, sequencing, mass spectroscopy, flow cytometry or any combinations thereof.
11. A set of polynucleotides for detecting a set comprising at least two miRNAs for diagnosing and/or prognosing of BPH in a blood sample from a patient.
12. The set of polynucleotides according to item 11, wherein the blood sample is a blood cell sample or a leukocyte containing sample.
13. The set of polynucleotides according to item 11 or 12, wherein the miRNAs are selected from the miRNAs that are differentially regulated in blood samples from BPH patients as compared to prostate cancer patients listed in FIG. 3 or FIG. 8
14. The set of polynucleotides according to any of the items 11, 12 or 13, wherein the set of miRNAs that are differentially regulated in blood samples from BPH patients as compared to prostate cancer patients is selected from the sets of miRNAs listed in FIG. 5 or FIG. 11
15. The set of polynucleotides according to item 11 or 12, wherein the miRNAs are selected from the miRNAs that are differentially regulated in blood samples from BPH patients as compared to healthy controls listed in FIG. 2 or FIG. 7
16. The set of polynucleotides according to any of the items 11, 12 or 15, wherein the set of miRNAs that are differentially regulated in blood samples from BPH patients as compared to healthy controls is selected from the sets of miRNAs listed in FIG. 4 or FIG. 10.
17. The set of polynucleotides according to any one of items 11 to 16, wherein the set of miRNAs representative for diagnosis of BPH comprises at least 1, 7, 10, 15, 20, 25, 30, 35, 40, 50, 75, 100 of miRNAs.
18. Use of set of polynucleotides according to any of the items 11 to 17 for diagnosing and/or prognosing BPH in a patient.
19. A set of primer pairs for determining the expression level of a set of miRNAs in a blood sample of a patient for diagnosing and/or prognosing BPH.
20. The set of primer pairs according to item 19, wherein the blood sample is a blood cell sample or a leukocyte containing sample.
21. The set of primer pairs according to item 19 or 20, wherein the miRNAs are selected from the miRNAs that are differentially regulated in blood samples from BPH patients as compared to prostate cancer patients listed in FIG. 3 or FIG. 8.
22. The set of primer pairs according to any of the items 19, 20 or 21, wherein the set of miRNAs comprises at least one set of miRNAs listed in FIG. 5 or FIG. 11.
23. The set of primer pairs according to item 19 or 20, wherein the miRNAs are selected from the miRNAs that are differentially regulated in blood samples from BPH patients as compared to healthy controls listed in FIG. 2 or FIG. 7.
24. The set of primer pairs according to any of the items 19, 20 or 23, wherein the set of miRNAs that are differentially regulated in blood samples from BPH patients as compared to healthy controls comprises at least one set of miRNAs listed in FIG. 4 or FIG. 10.
25. The set of primer pairs according to any one of items 19 to 24, wherein the set of miRNAs representative for diagnosis of BPH comprises at least 1, 7, 10, 15, 20, 25, 30, 35, 40, 50, 75, 100 of miRNAs.
26. Use of set of primer pairs according to any of the items 19 to 25 for diagnosing and/or prognosing BPH in a patient.
27. Means for diagnosing and/or prognosing of BPH in a blood sample of a subject comprising:
   (a) a set of at least two polynucleotides according to any of the items 11 to 17 or
   (b) a set of at least two primer pairs according to any of the items 19 to 25
28. A kit for diagnosing and/or predicting BPH, comprising:
   (a) means for determining the miRNA expression profile of a RNA sample of a subject, and
   (b) at least one reference expression profile for a particular condition.
29. The kit according to item 28 comprising the means according to item 27
30. A set of miRNAs isolated from a blood sample from a subject for diagnosing and/or prognosing of BPH, wherein the miRNAs are selected from the miRNAs as indicated in any one of items 4 to 8.
31. The set of miRNAs according to item 30, wherein the blood sample is a blood cell sample or a leukocyte containing sample.
32. The set of miRNAs according to any of the items 30 or 31, wherein the set of miRNAs comprises miRNAs that are differentially regulated in blood samples from BPH patients as compared to prostate cancer patients and wherein the miRNAs are selected from FIG. 3 or FIG. 8
33. The set of miRNAs according to any of the items 30, 31 or 32, wherein the set of miRNAs comprises at least one set of miRNAs listed in FIG. 5 or FIG. 11
34. The set of miRNAs according to any of the items 30 or 31, wherein the set of miRNAs comprises miRNAs that are differentially regulated in blood samples from BPH patients as compared to healthy controls and wherein the miRNAs are selected from FIG. 2 or FIG. 7
35. The set of miRNAs according to any of the items 30, 31 or 34, wherein the set of miRNAs comprises at least one set of miRNAs listed in FIG. 4 or FIG. 10
36. Use of a set of miRNAs according to any of the items 31 to 35 for diagnosing and/or prognosing of BPH in a subject, particularly a human subject.
37. Use of a set of miRNAs according to any of the items 30 to 33 for diagnosing and/or prognosing of BPH from prostate cancer in a subject, particularly a human subject.
38. Use of a set of miRNAs according to any of the items 30, 31, 34 or 35 for diagnosing and/or prognosing of BPH from healthy control in a subject, particularly a human subject.
39. The set of miRNAs of any of the items 30 to 35 bound to a carrier, e.g. a microarray.
40. A method of diagnosing prostate cancer, comprising the steps
   (a) determining an expression profile of a set of miRNAs, in a blood sample from a patient, and
   (b) comparing said expression profile to a reference
      wherein the comparison of said determined expression profile to said reference allows for the diagnosis of prostate cancer.
41. The method according to item 40, wherein the blood sample is a blood cell sample or a leukocyte containing sample.
42. The method according to item 40 or 41, wherein the set of miRNAs comprises miRNAs that are differentially regulated in blood samples from healthy controls as compared to prostate cancer patients and wherein the miRNAs are selected from FIG. 6 or FIG. 9
43. The method according to any of the items 40 to 42, wherein the set of miRNAs comprises at least one set of miRNAs listed in FIG. 12
44. The method according to any one of items 40 to 43 wherein the expression profile is determined by nucleic acid hybridization, nucleic acid amplification, polymerase extension, sequencing, mass spectroscopy, flow cytometry or any combinations thereof.
45. A set of polynucleotides for detecting a set comprising at least two miRNAs for diagnosing and/or prognosing of prostate cancer in a blood sample from a patient
46. The set of polynucleotides according to item 45, wherein the blood sample is a blood cell sample or a leukocyte containing sample.
47. The set of polynucleotides according to any of the items 45 or 46 wherein the miRNAs are selected from the miRNAs that are differentially regulated in blood samples from prostate cancer patients as compared to healthy controls listed in FIG. 6 or FIG. 9
48. The set of polynucleotides according to any of the items 45 to 47, wherein the set of miRNAs comprises at least one set of miRNAs listed in FIG. 12.
49. Means for diagnosing and/or prognosing of prostate cancer in a blood sample of a subject comprising:
   (a) a set of at least two polynucleotides according to any of the items 45 to 48 or
   (b) a set of at least two primer pairs for detecting of at least a miRNA listed in FIG. 6 or FIG. 9
50. A kit for diagnosing and/or prognosing prostate cancer, comprising:
   (a) means according to item 49 for determining the miRNA expression profile of a RNA sample of a subject, and
   (b) at least one reference
51. A set of miRNAs isolated from a blood sample from a subject for diagnosing and/or prognosing of prostate cancer
52. The set of miRNAs according to item 51, wherein the blood sample is a blood cell sample or a leukocyte containing sample.
53. The set of miRNAs according to any of the items 51 or 52, wherein the set of miRNAs comprises miRNAs that are differentially regulated in blood samples from prostate cancer patients as compared to healthy controls and wherein the miRNAs are selected from FIG. 6 or FIG. 9
54. The set of miRNAs according to any of the items 51 to 53, wherein the set of miRNAs comprises at least one set of miRNAs listed in FIG. 4 or FIG. 10
55. Use of a set of miRNAs according to any of the items 51 to 54 for diagnosing and/or prognosing of prostate cancer in a subject, particularly a human subject.

### BRIEF DESCRIPTION OF THE DRAWINGS

FIG. 1:

Overview of miRNA sequences suitable for diagnosing and/or prognosing prostate diseases.

FIG. 2:

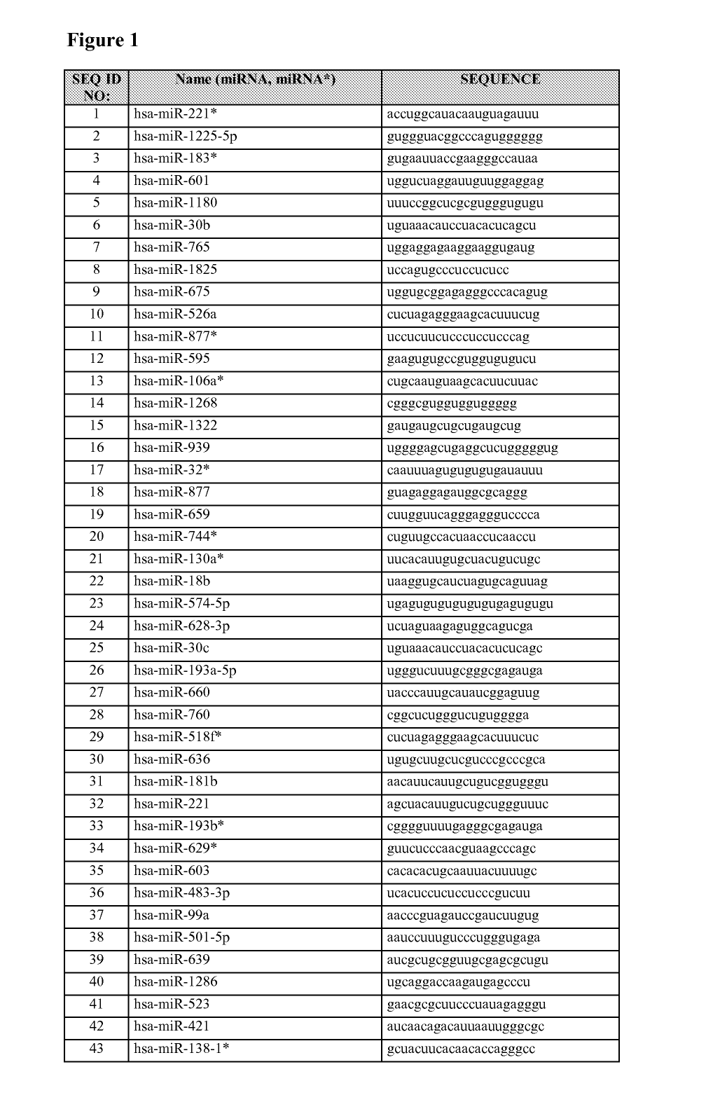

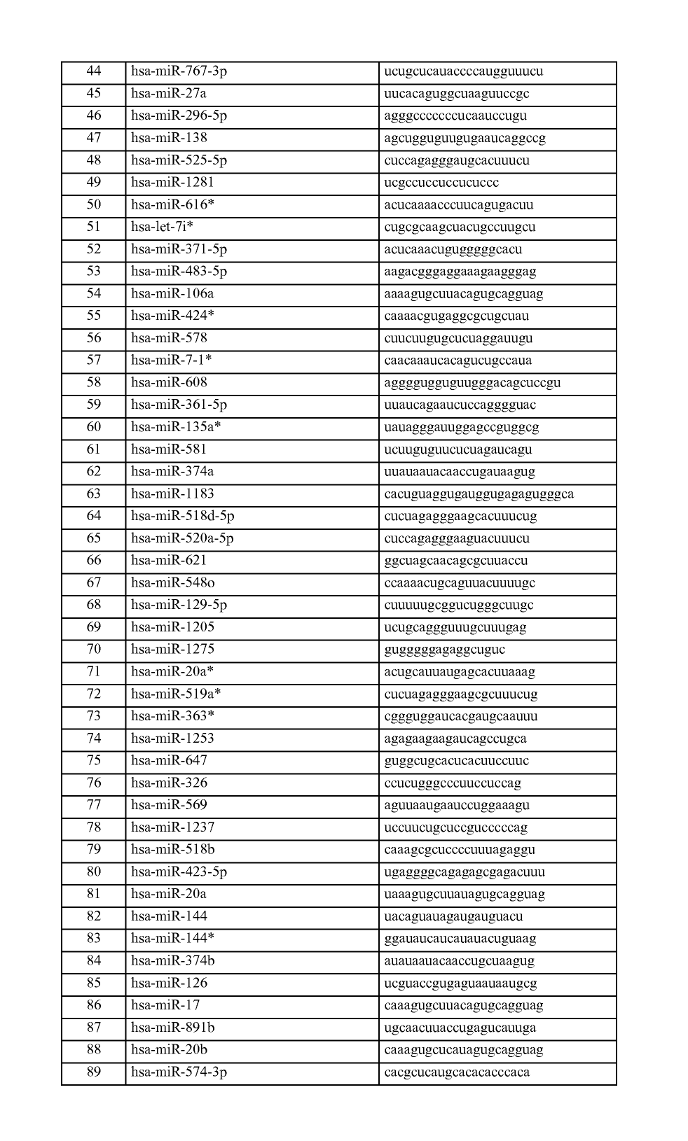

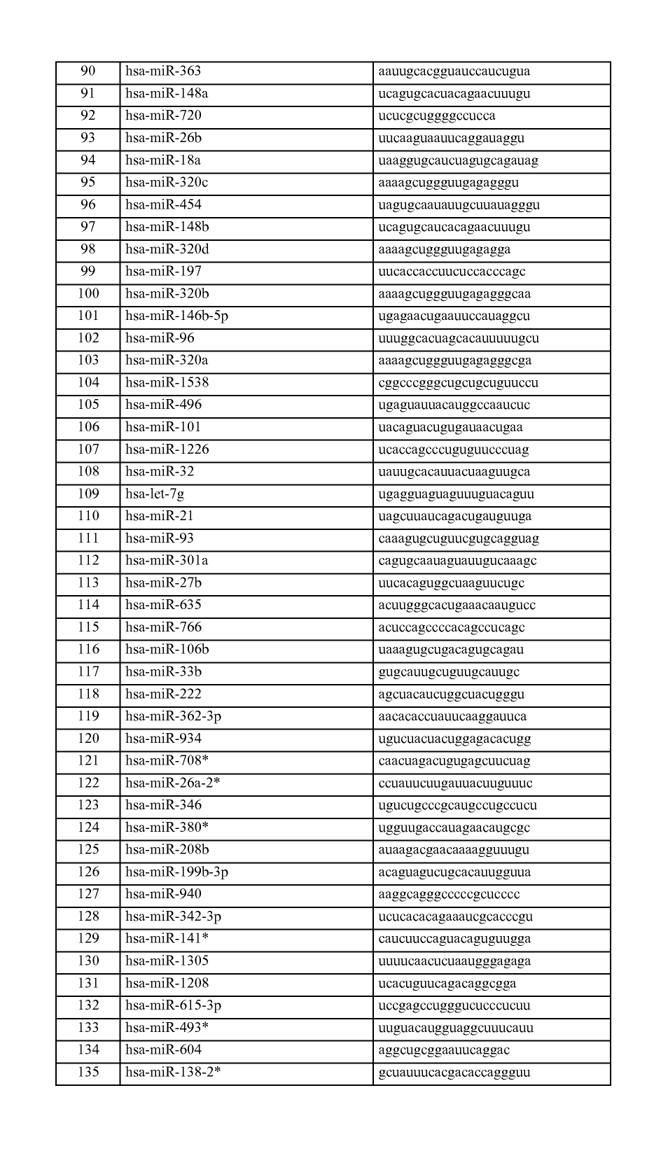

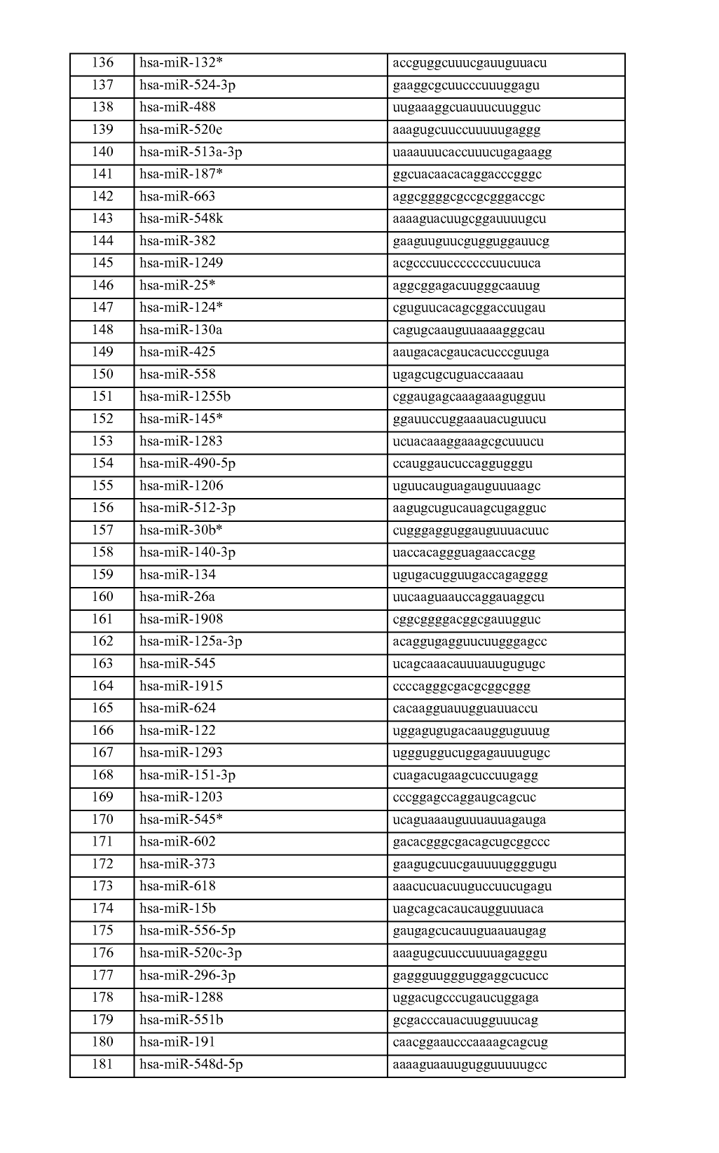

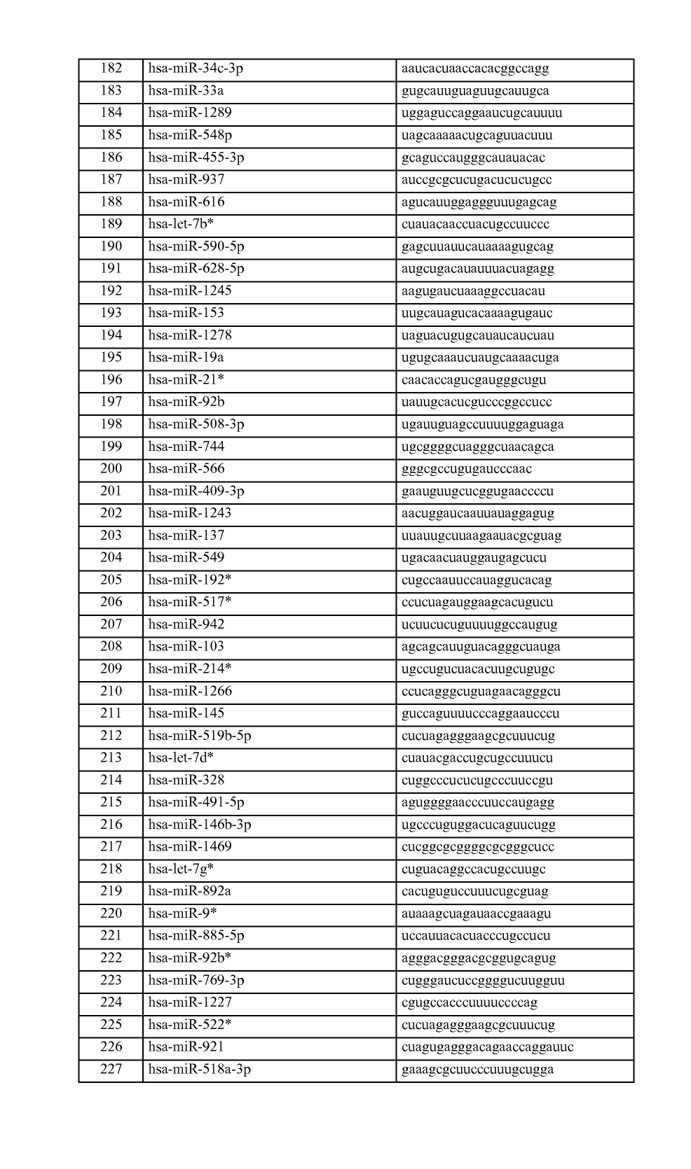

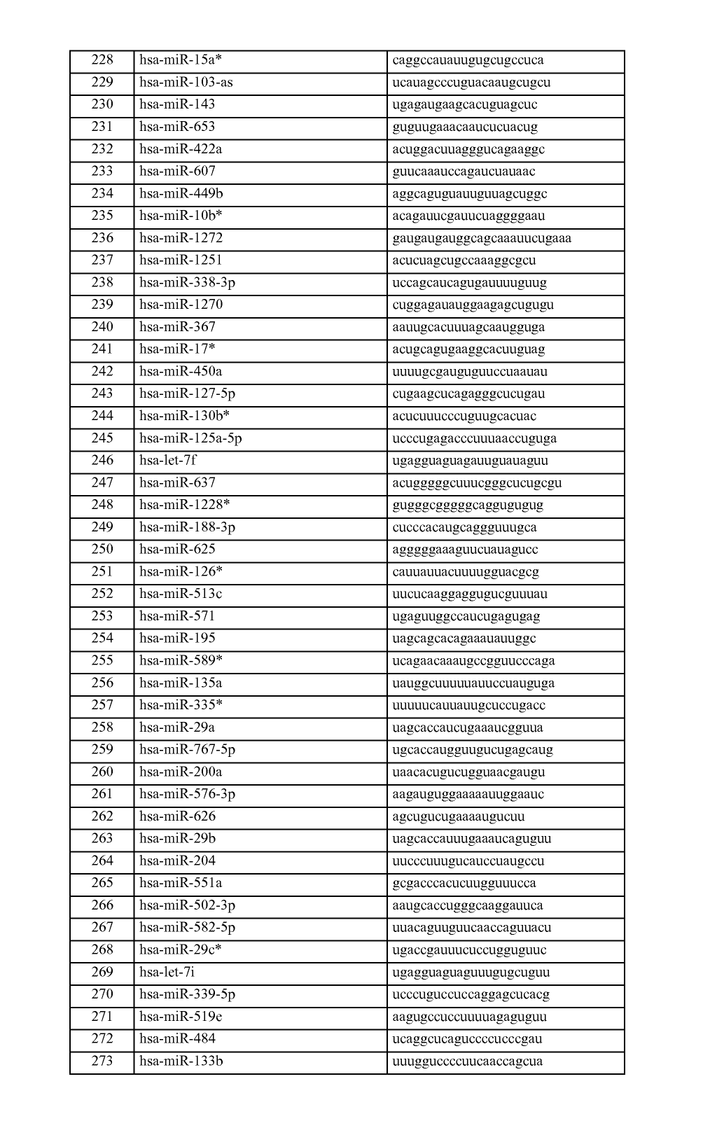

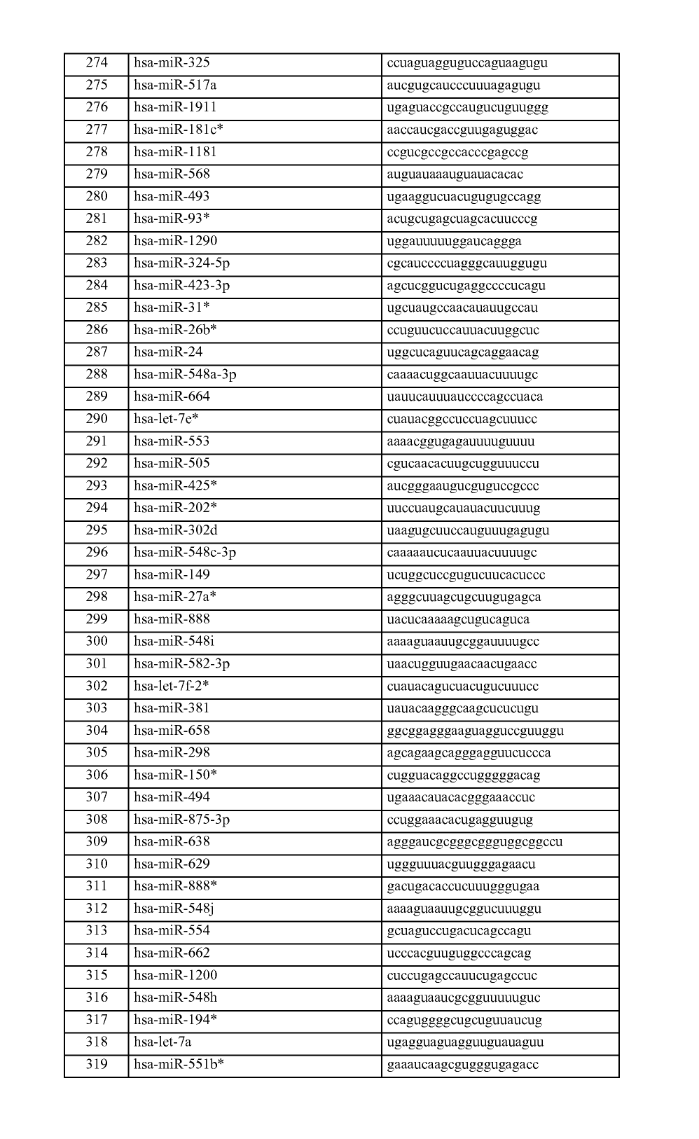

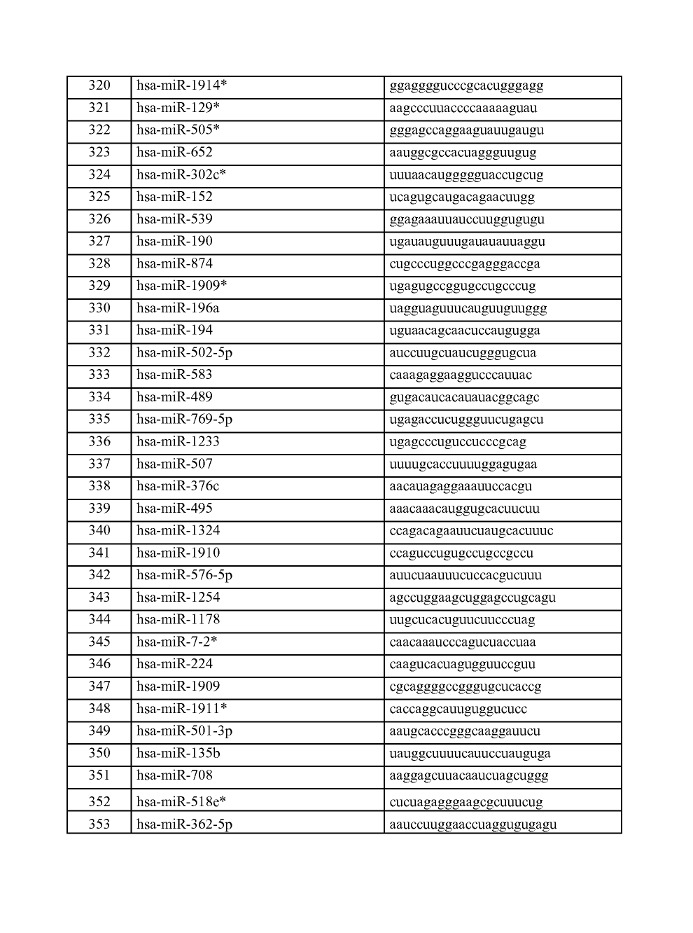

Overview of miRNAs that are found to be differentially regulated between healthy controls and subjects suffering from BPH. Experimental details: SEQ ID NO: sequence identification number, miRNA: identifier of the miRNA according to miRBase, median g1: median intensity obtained from microarray analysis for healthy controls in counts/sec, median g2: median intensity obtained from microarray analysis for individuals with BPH in counts/sec, qmedian: ratio of median g1/median g2, log qmedian: log of qmedian, ttest_rawp: p-value obtained when applying t-test, ttest_adjp: adjusted p-value in order to reduce false discovery rate by Benjamini-Hochberg adjustment, AUC: Area under the curve, limma_rawp: p-value obtained when applying limma-test, limma_adjp: adjusted p-value in order to reduce false discovery rate by Benjamini-Hochberg adjustment.

FIG. 3:

Overview of miRNAs that are found to be differentially regulated between prostate cancer subjects and subjects suffering from BPH. Experimental details: SEQ ID NO: sequence identification number, miRNA: identifier of the miRNA according to miRBase, median g1: median intensity obtained from microarray analysis for prostate cancer subjects in counts/sec, median g2: median intensity obtained from microarray analysis for individuals with BPH in counts/sec, qmedian: ratio of median g1/median g2, log qmedian: log of qmedian, ttest_rawp: p-value obtained when applying t-test, ttest_adjp: adjusted p-value in order to reduce false discovery rate by Benjamini-Hochberg adjustment, AUC: Area under the curve, limma_rawp: p-value obtained when applying limma-test, limma_adjp: adjusted p-value in order to reduce false discovery rate by Benjamini-Hochberg adjustment.

FIG. 4:

Sets of miRNAs (miRNA signatures SNB-1 to SNB-753) that allow for effective diagnosis and/or prognosis of BPH when differentiating BPH and healthy controls. Experimental details: SEQ ID NO: sequence identification number, miRNA: identifier of the miRNA according to miRBase, Acc=accuracy, Spec=specificity, Sens=sensitivity

FIG. 5:

Sets of miRNAs (miRNA signatures SPB-1 to SPB-247) that allow for effective diagnosis and/or prognosis of BPH when differentiating prostate cancer and BPH. Experimental details: SEQ ID NO: sequence identification number, miRNA: identifier of the miRNA according to miRBase, Acc=accuracy, Spec=specificity, Sens=sensitivity.

FIG. 6:

Overview of miRNAs that are found to be differentially regulated between healthy controls and subjects suffering from prostate cancer. Experimental details: SEQ ID NO: sequence identification number, miRNA: identifier of the miRNA according to miRBase, median g1: median intensity obtained from microarray analysis for healthy controls in counts/sec, median g2: median intensity obtained from microarray analysis for individuals with prostate cancer in counts/sec, qmedian: ratio of median g1/median g2, log qmedian: log of qmedian, ttest_rawp: p-value obtained when applying t-test, ttest_adjp: adjusted p-value in order to reduce false discovery rate by Benjamini-Hochberg adjustment, AUC: Area under the curve, limma_rawp: p-value obtained when applying limma-test, limma_adjp: adjusted p-value in order to reduce false discovery rate by Benjamini-Hochberg adjustment.

FIG. 7:

Overview of further miRNAs that are found to be differentially regulated between 68 healthy controls and 33 subjects suffering from BPH. Experimental details: SEQ ID NO: sequence identification number, miRNA: identifier of the miRNA according to miRBase, median g1: median intensity obtained from microarray analysis for healthy controls in counts/sec, median g2: median intensity obtained from microarray analysis for individuals with BPH in counts/sec, qmedian: ratio of median g1/median g2, log qmedian: log 2 of qmedian, ttest_adjp: p-value obtained when applying t-test, adjusted p-value in order to reduce false discovery rate by Benjamini-Hochberg adjustment.

FIG. 8:

Overview of further miRNAs that are found to be differentially regulated between 64 prostate cancer subjects and 33 subjects suffering from BPH. Experimental details: SEQ ID NO: sequence identification number, miRNA: identifier of the miRNA according to miRBase, median g1: median intensity obtained from microarray analysis for individuals with BPH in counts/sec, median g2: median intensity obtained from microarray analysis for individuals with prostate cancer in counts/sec, qmedian: ratio of median g1/median g2, log qmedian: log 2 of qmedian, ttest_adjp: p-value obtained when applying t-test, adjusted p-value in order to reduce false discovery rate by Benjamini-Hochberg adjustment.

FIG. 9:

Overview of further miRNAs that are found to be differentially regulated between 68 healthy controls and 64 subjects suffering from prostate cancer. Experimental details: SEQ ID NO: sequence identification number, miRNA: identifier of the miRNA according to miRBase, median g1: median intensity obtained from microarray analysis for healthy controls in counts/sec, median g2: median intensity obtained from microarray analysis for individuals with prostate cancer in counts/sec, qmedian: ratio of median g1/median g2, log qmedian: log 2 of qmedian, ttest_adjp: p-value obtained when applying t-test, adjusted p-value in order to reduce false discovery rate by Benjamini-Hochberg adjustment.

FIG. 10:

Further sets of miRNAs (miRNA signatures SNB-754 to SNB-968) that allow for effective diagnosis and/or prognosis of BPH when differentiating BPH and healthy controls. Experimental details: SEQ ID NO: sequence identification number, miRNA: identifier of the miRNA according to miRBase.

FIG. 11:

Further sets of miRNAs (miRNA signatures SPB-248 to SPB-356) that allow for effective diagnosis and/or prognosis of BPH when differentiating prostate cancer and BPH. Experimental details: SEQ ID NO: sequence identification number, miRNA: identifier of the miRNA according to miRBase.

FIG. 12:

Sets of miRNAs (miRNA signatures SNP-1 to SNP-226) that allow for effective diagnosis and/or prognosis of prostate cancer when differentiating prostate cancer and healthy controls. Experimental details: SEQ ID NO: sequence identification number, miRNA: identifier of the miRNA according to miRBase.

### EXAMPLES

The Examples are designed in order to further illustrate the present invention and serve a better understanding. They are not to be construed as limiting the scope of the invention in any way.

Materials and Methods

miRNA Extraction and Microarray Screening

Blood of patients has been extracted as previously described [1]. In brief, 2.5 to 5 ml blood was extracted in PAXgene Blood RNA tubes (BD, Franklin Lakes, N.J. USA) and centrifuged at 5000×g for 10 min at room temperature. The miRNeasy kit (Qiagen GmbH, Hilden) was used to isolate total RNA including miRNA from the resuspended pellet according to manufacturer's instructions. The eluted RNA was stored at −70° C.

All samples were shipped overnight on dry ice and analyzed with the Geniom RT Analyzer (febit biomed GmbH, Heidelberg, Germany) at the in-house genomic service department using the Geniom Biochip miRNA Homo sapiens. Each array contains 7 replicates of about 863 miRNAs and miRNA star sequences as annotated in the Sanger miRBase releases 12.0, 13.0 and 14.0. On-chip sample labeling with biotin was carried out by micro fluidic-based primer extension labeling of miRNAs (MPEA [2]). Following hybridization for 16 hours at 42° C., the biochip was washed and a program for signal enhancement was carried out. All steps from sample loading to miRNA detection were processed without any manual intervention and inside the machine. The detection pictures were evaluated using the Geniom Wizard Software. For each feature, the median signal intensity was calculated. Following a background correction step, the median of the 7 replicates of each miRNA was computed. To normalize the data across different arrays, quantile normalization [3] was applied and all further analyses were carried out using the normalized and background subtracted intensity values. Since the miRBase has been upgraded twice in the past year from version 12.0 to 14, we used for the final data analysis the 863 miRNAs that were consistently present in all three versions.

Statistical Analysis

To estimate the value of single miRNAs, t-tests (unpaired, two-tailed) were carried out. The resulting p-values have been adjusted for multiple testing by Benjamini-Hochberg adjustment [4, 5]. In addition to this single biomarker analysis, we performed supervised classification of samples by using Support Vector Machines (SVM [6]) as implemented in the R e1071 package [7]. As parameters, we evaluated different kernel methods including linear, polynomial (degree 2 to 5), sigmoid and radial basis function kernels. The cost parameter was sampled from 0.01 to 10 in decimal powers. As subset selection technique, a filter approach based on t-test was carried out. In each iteration, the s miRNAs with lowest p-values were computed on the training set in each fold of a standard 10-fold cross validation, where s was sampled in regular intervals between 2 and 300. The respective subset was used to train the SVM and to carry out the prediction of the test samples in the cross validation. To compute probabilities for classes instead of class labels, a regression approach based on the output of the support vectors has been applied. To test for overtraining, non-parametric permutation tests have been applied. All computations were carried out using R [7], a freely available language for statistical tasks.

### REFERENCES

1. Keller A, Leidinger P, Borries A, Wendschlag A, Wucherpfennig F, Scheffler M, Huwer H, Lenhof H P, Meese E: miRNAs in lung cancer—studying complex fingerprints in patient's blood cells by microarray experiments. BMC Cancer 2009, 9:353.
2. Vorwerk S, Ganter K, Cheng Y, Hoheisel J, Stahler P F, Beier M: Microfluidic-based enzymatic on-chip labeling of miRNAs. N Biotechnol 2008, 25(2-3):142-149.
3. Bolstad B M, Irizarry R A, Astrand M, Speed T P: A comparison of normalization methods for high density oligonucleotide array data based on variance and bias. Bioinformatics 2003, 19(2):185-193.
4. Benjamini Y, Drai D, Elmer G, Kafkafi N, Golani I: Controlling the false discovery rate in behavior genetics research. Behav Brain Res 2001, 125(1-2):279-284.
5. Hochberg Y: A sharper bonferroni procedure for multiple tests of significance. Biometrica 1988, 75:185-193.
6. Vapnik V: The nature of statistical learning theory., 2nd edition edn. New York: Spinger; 2000.
7. Team R: R: A Language and Environment for Statistical Computing. In. Vienna: R Foundation for Statistical Computing; 2008.

1. A method of diagnosing BPH, comprising the steps
(a) determining an expression profile of a set of miRNAs, in a blood sample from a patient, and
(b) comparing said expression profile to a reference
wherein the comparison of said determined expression profile to said reference allows for the diagnosis of BPH.

2. The method according to claim 1, wherein the blood sample is a blood cell sample or a leukocyte containing sample.

3. The method according to claim 1, wherein the set of miRNAs comprises miRNAs that are differentially regulated in blood samples from BPH patients as compared to prostate cancer patients and wherein the miRNAs are selected from FIG. 3 or FIG. 8

4. The method according to claim 3, wherein the set of miRNAs comprises at least one set of miRNAs listed in FIG. 5 or FIG. 11

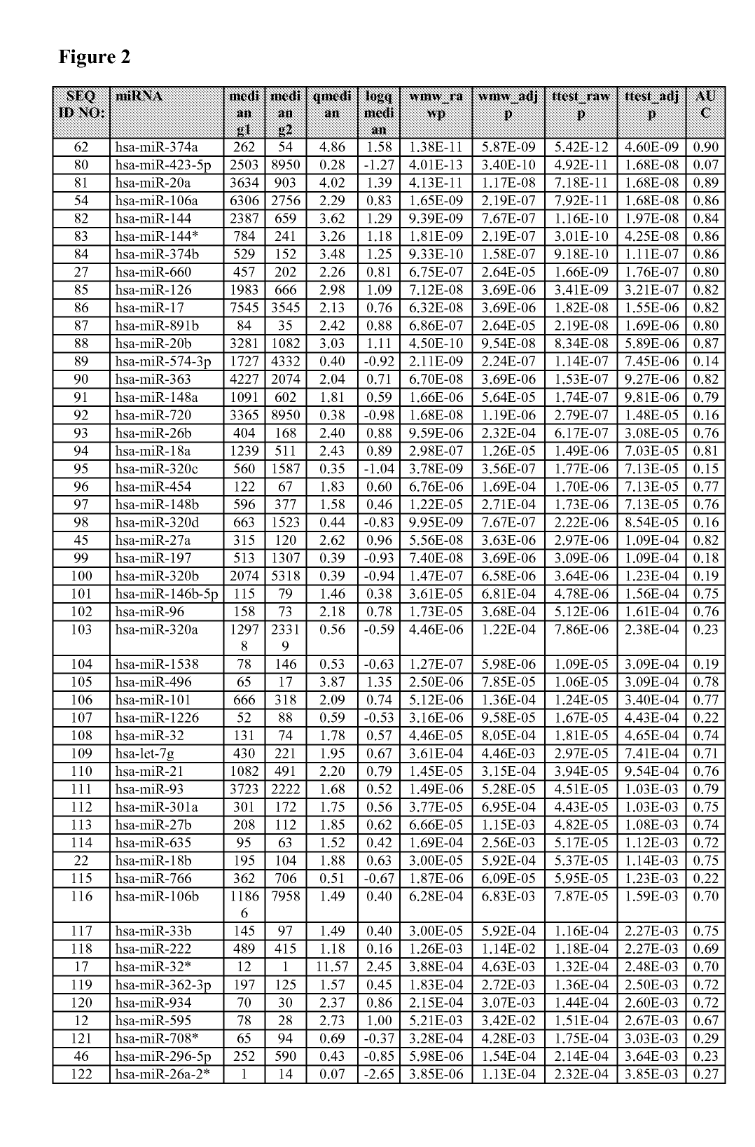

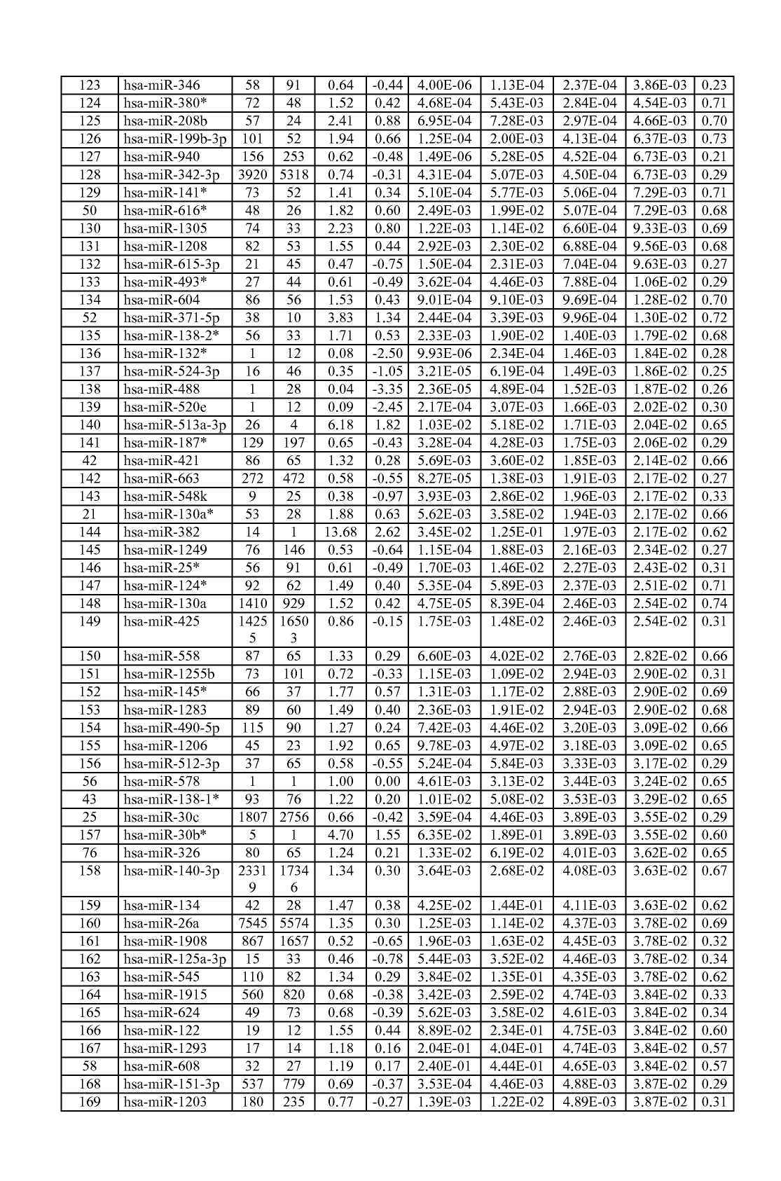

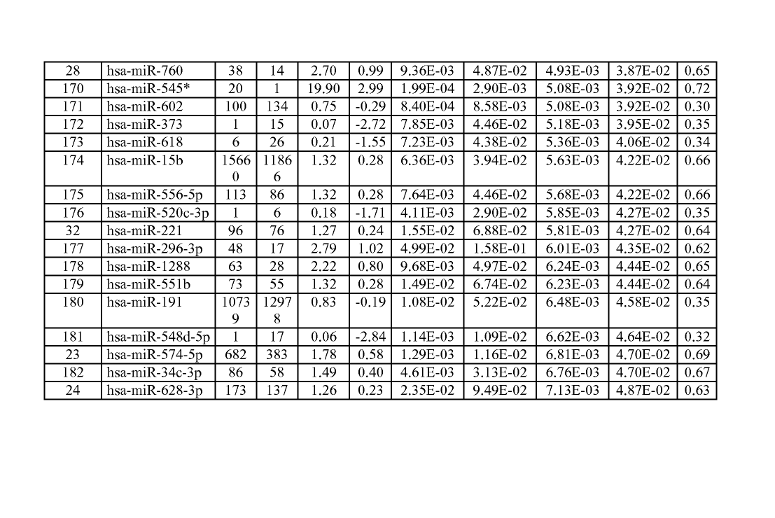

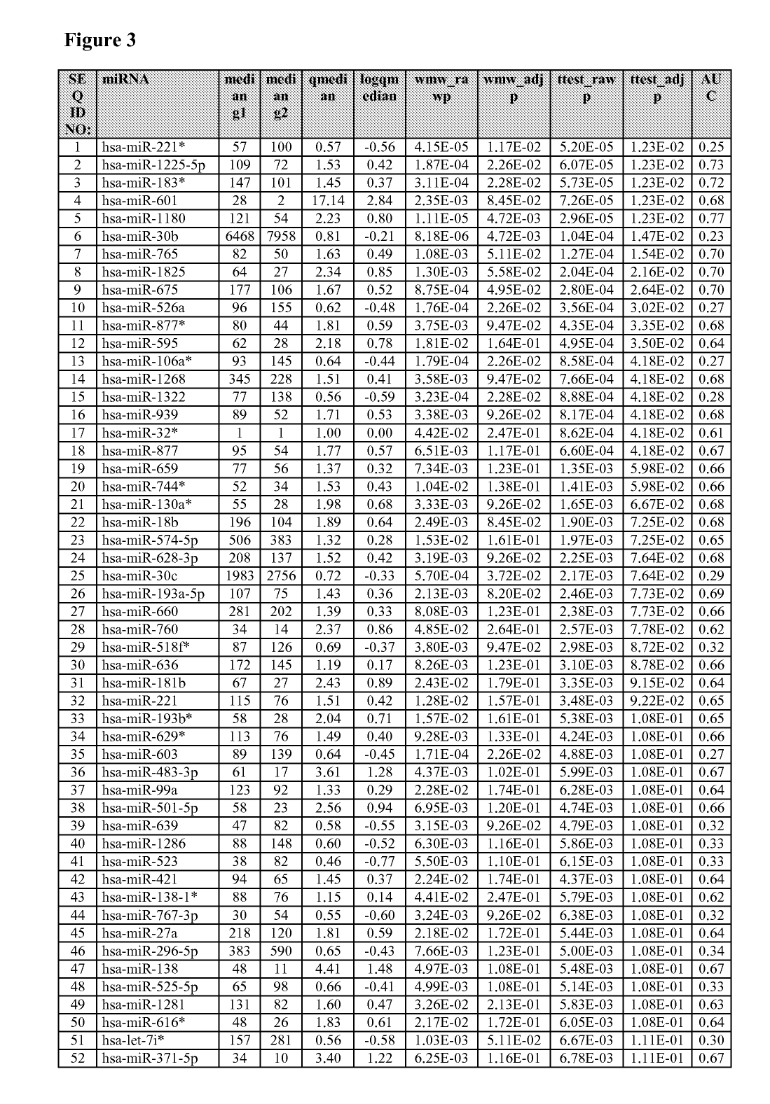

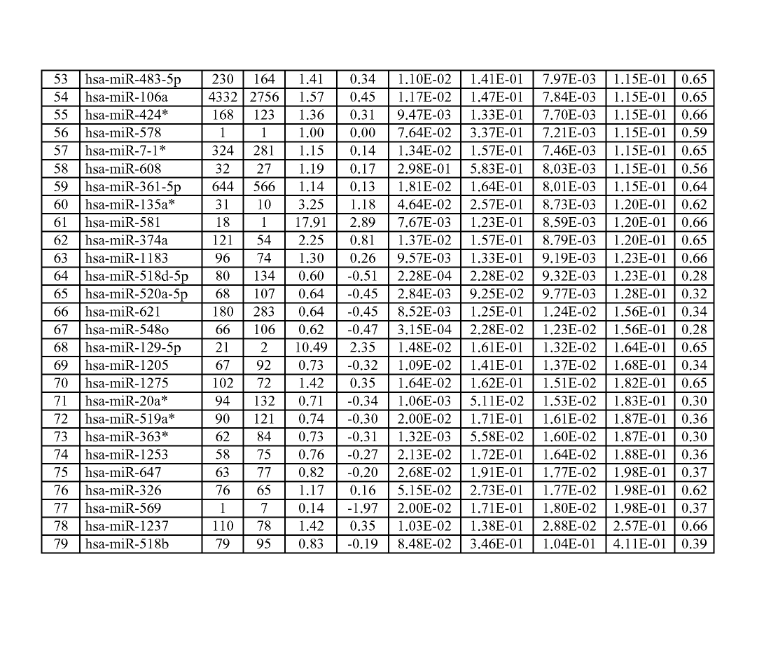

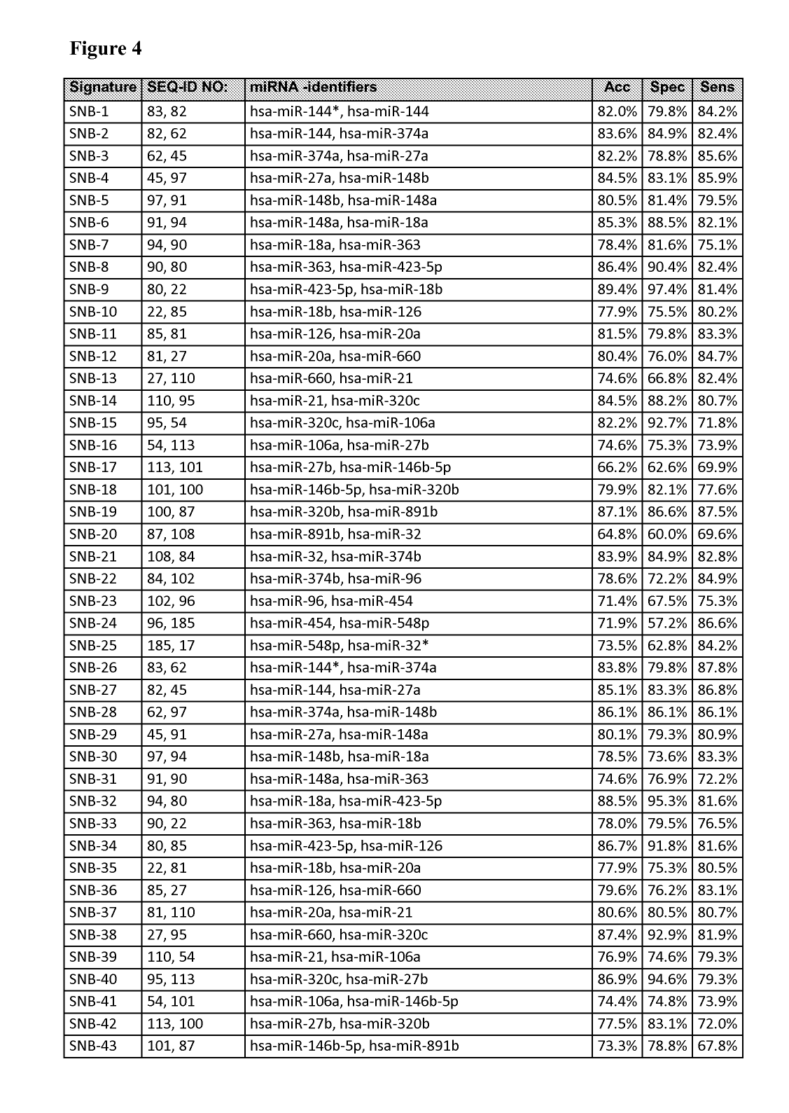

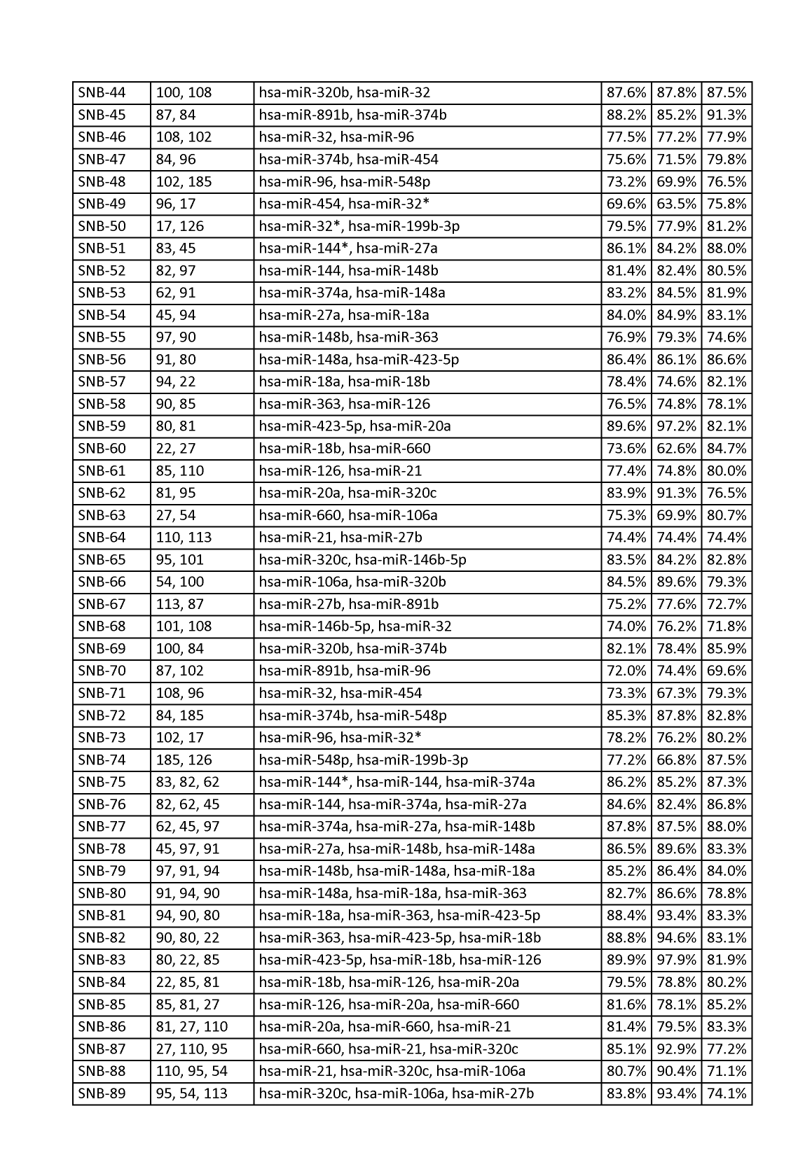

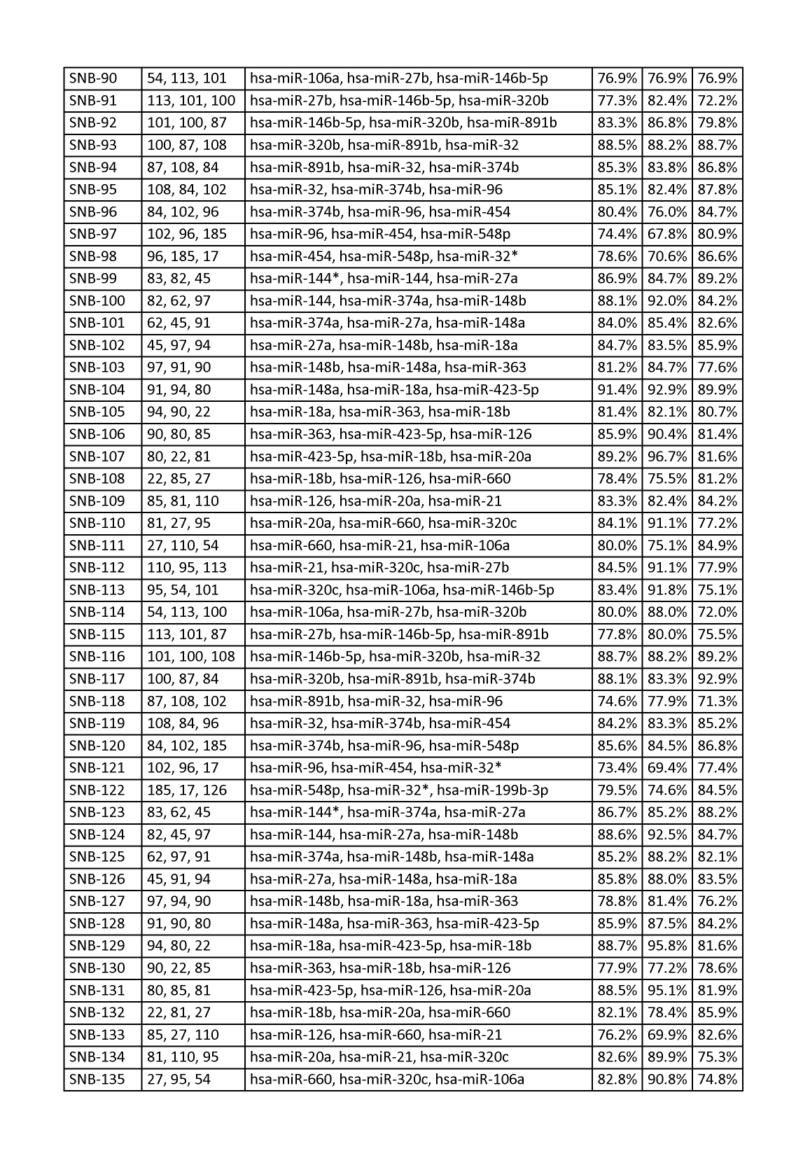

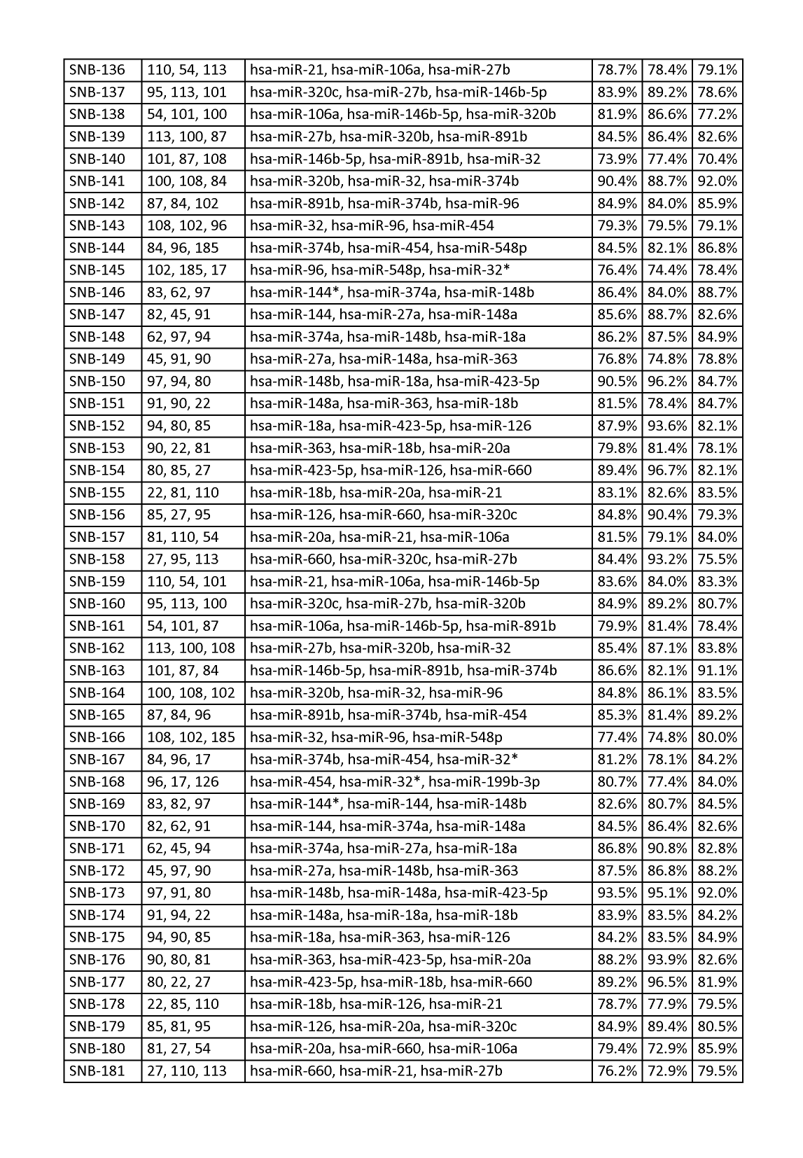

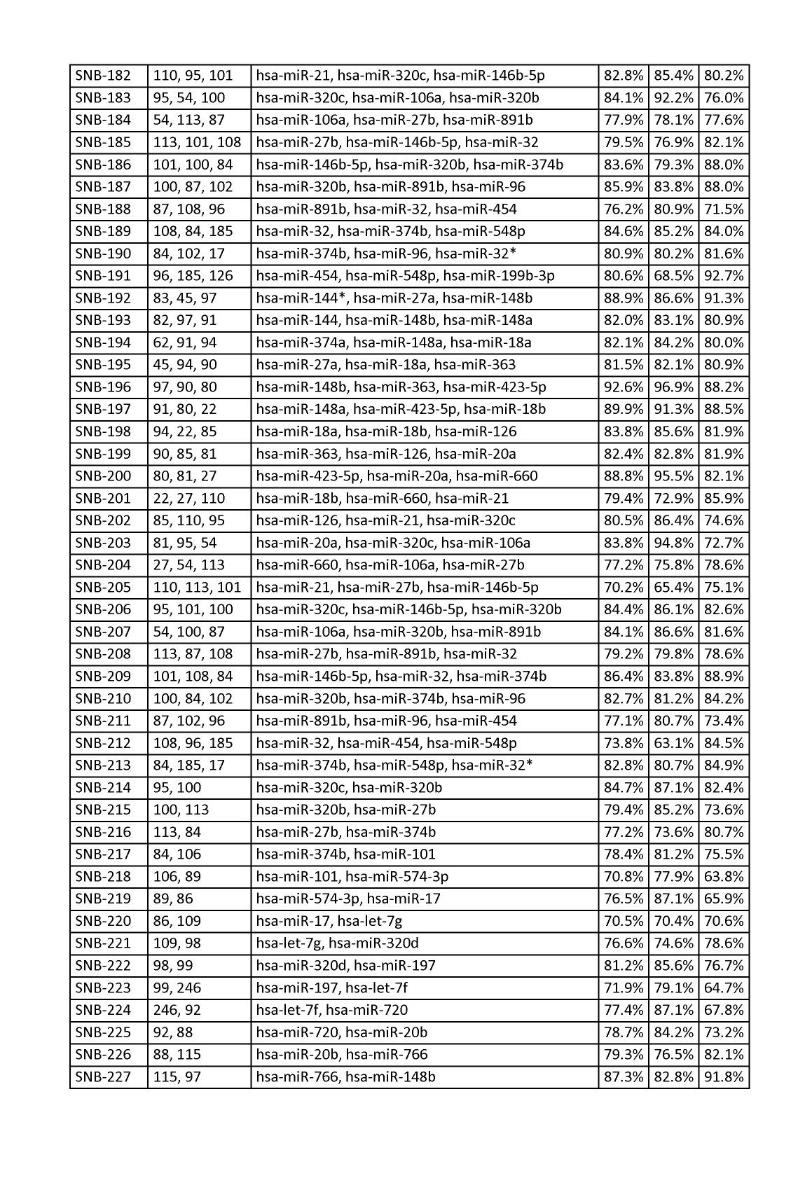

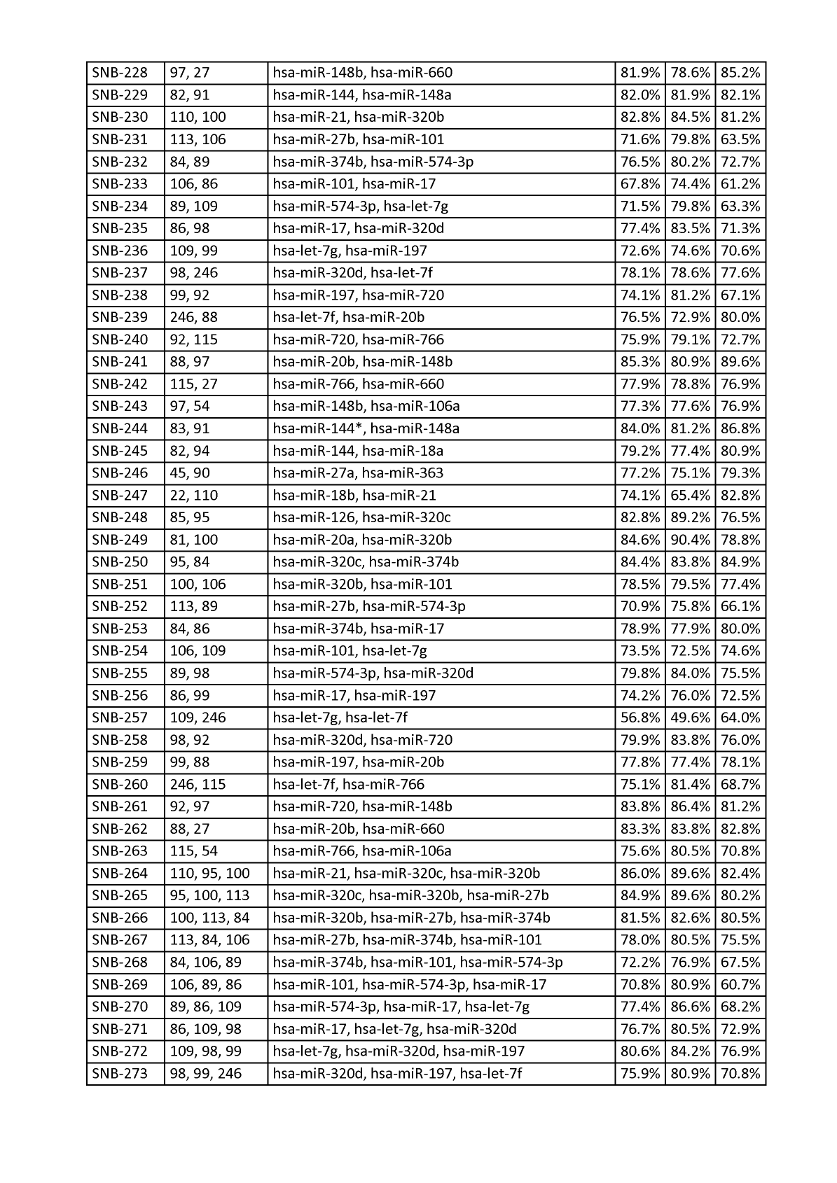

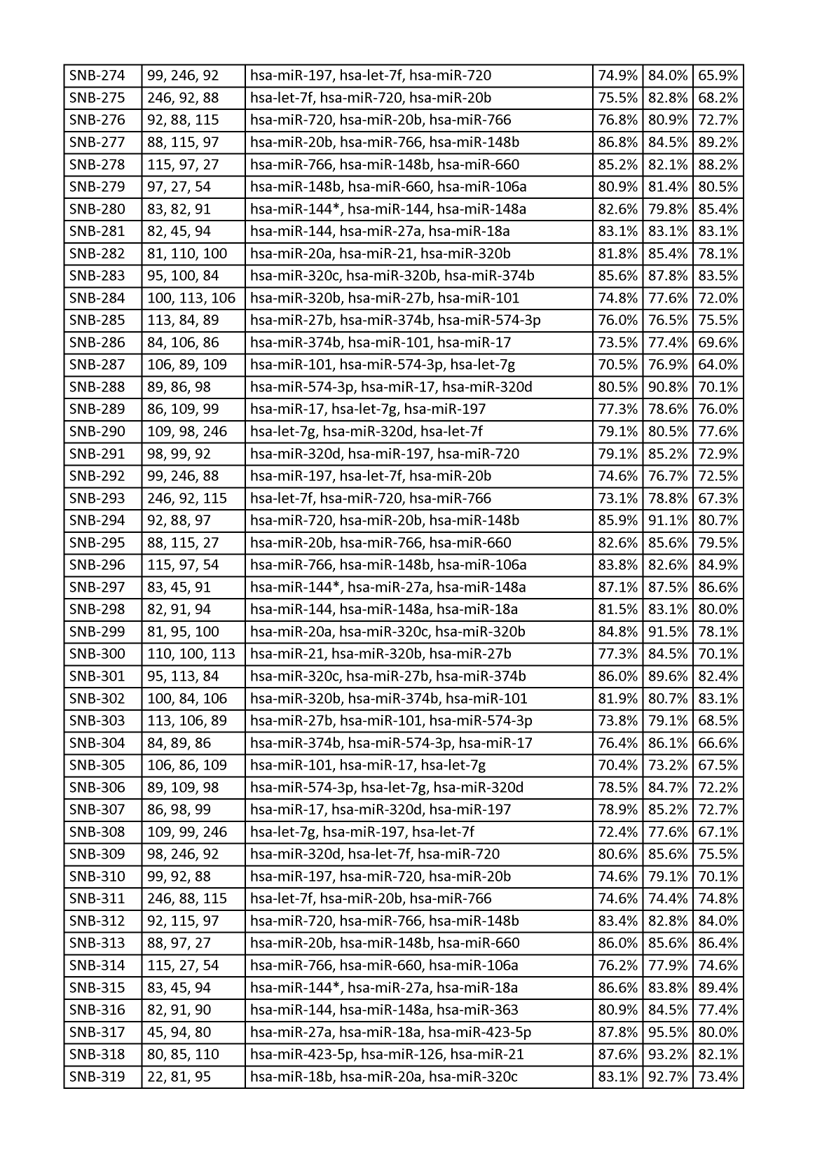

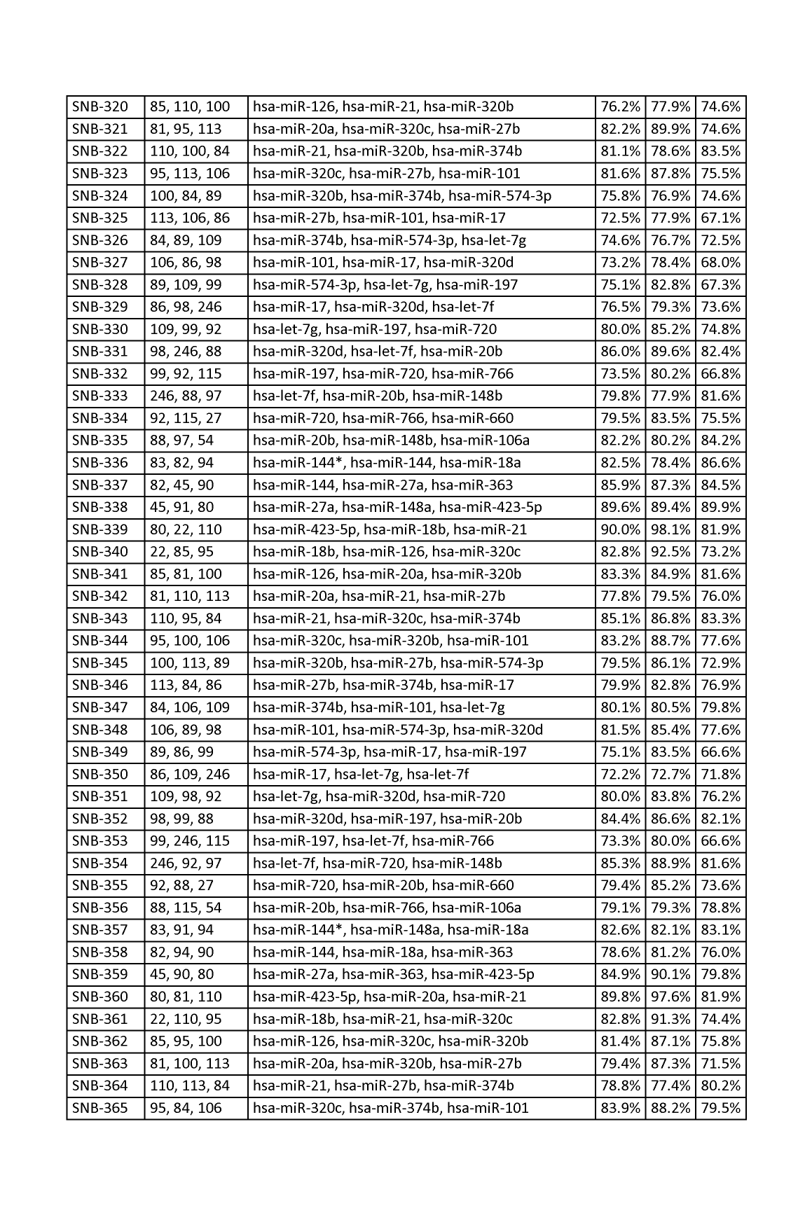

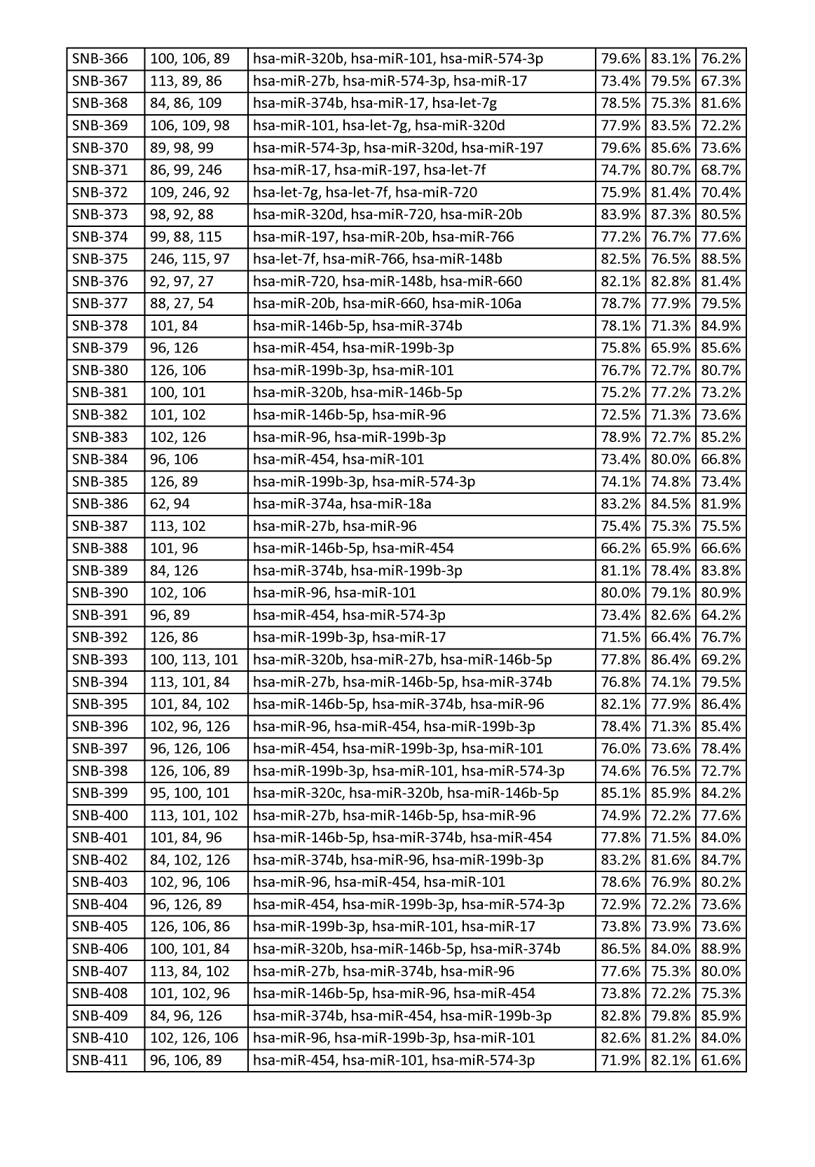

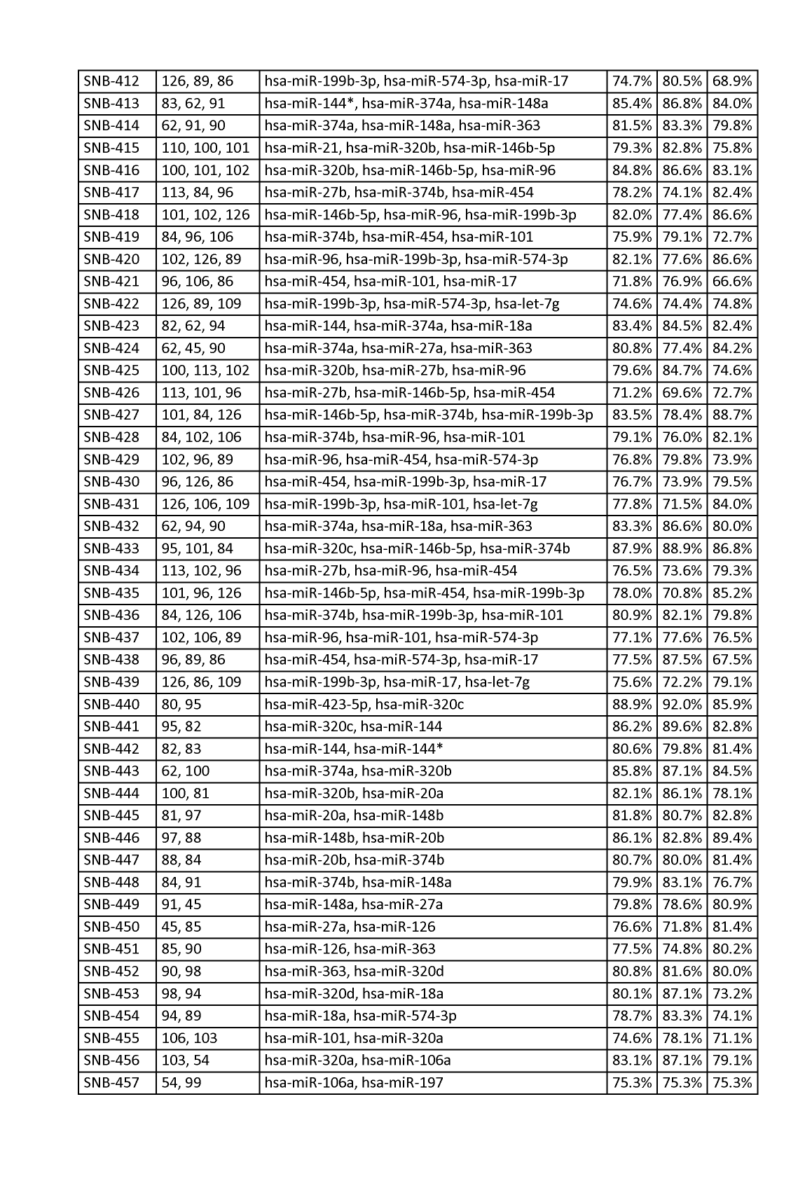

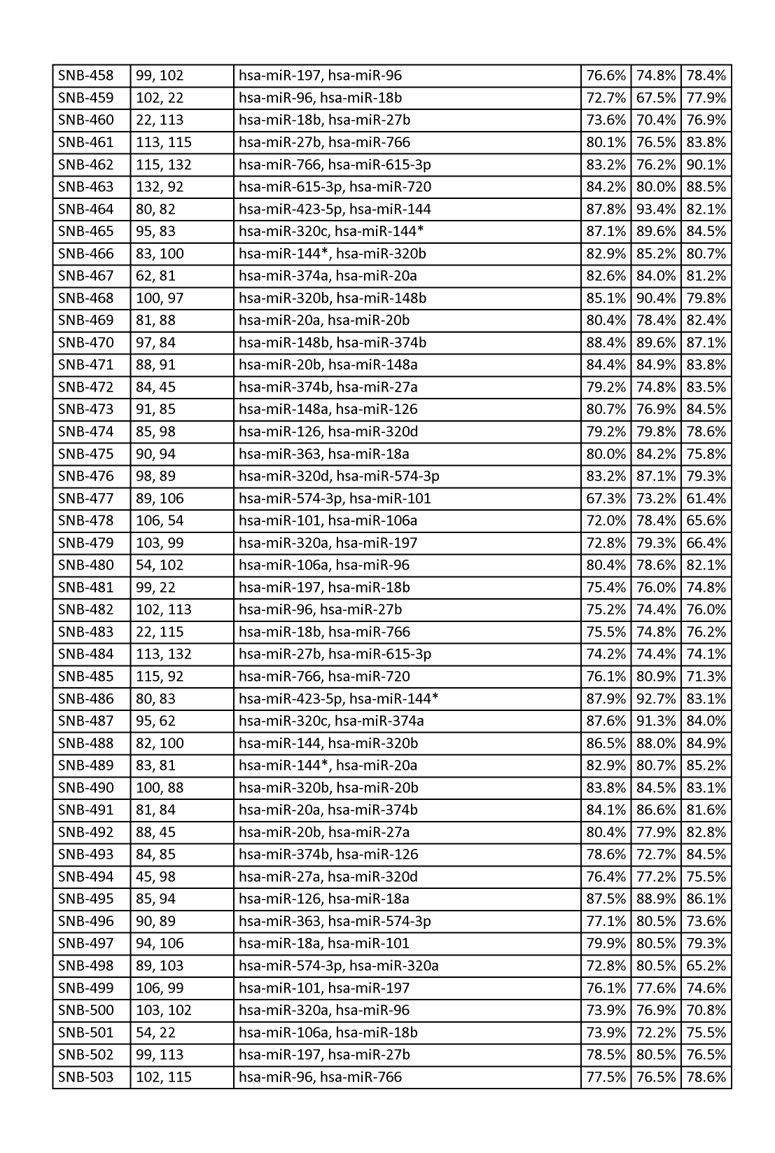

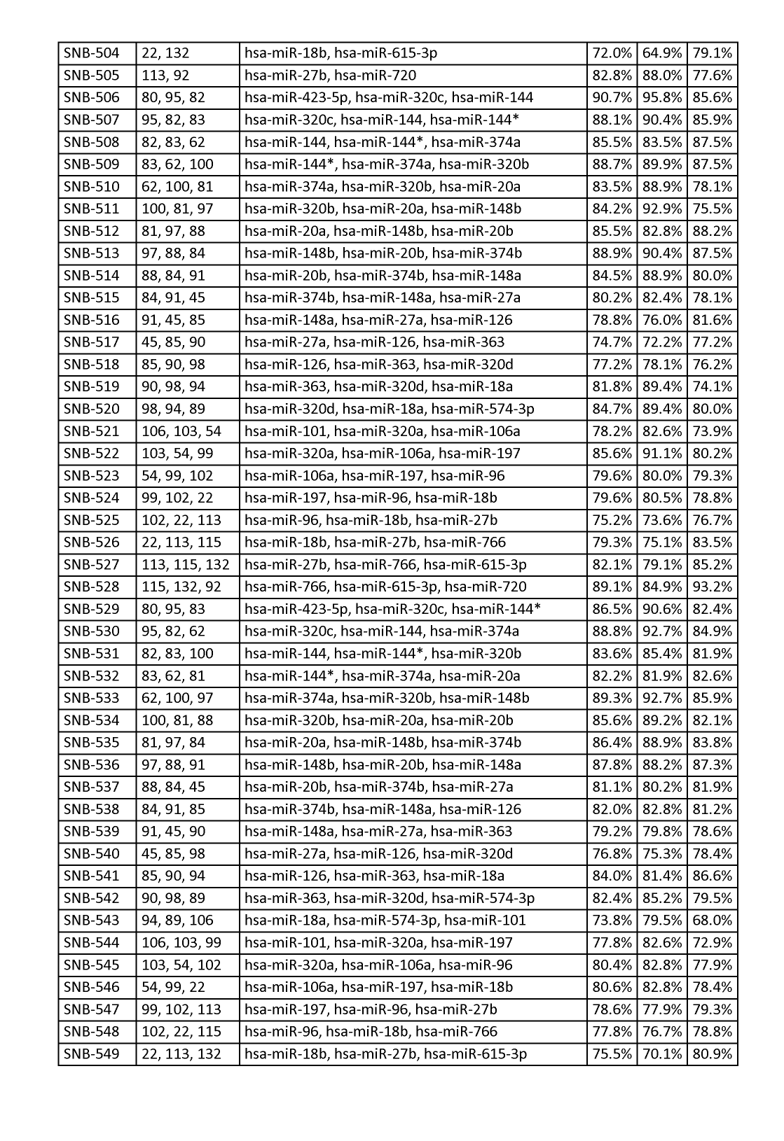

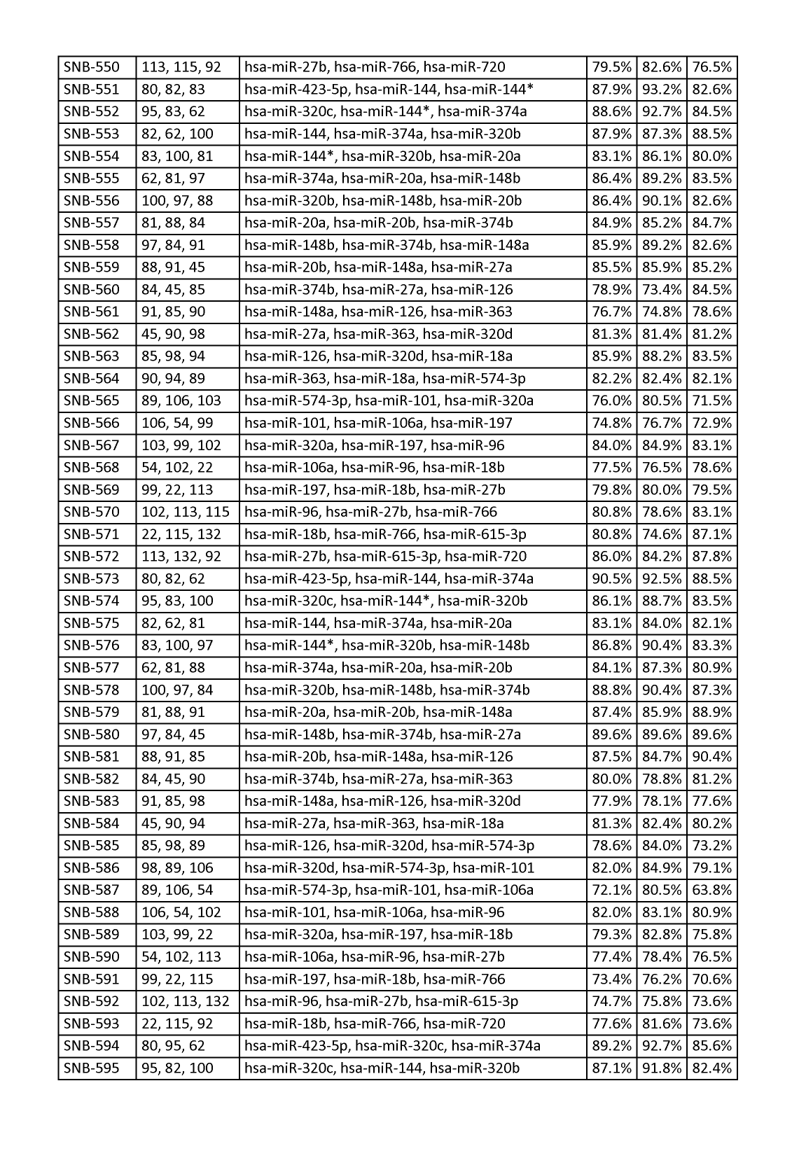

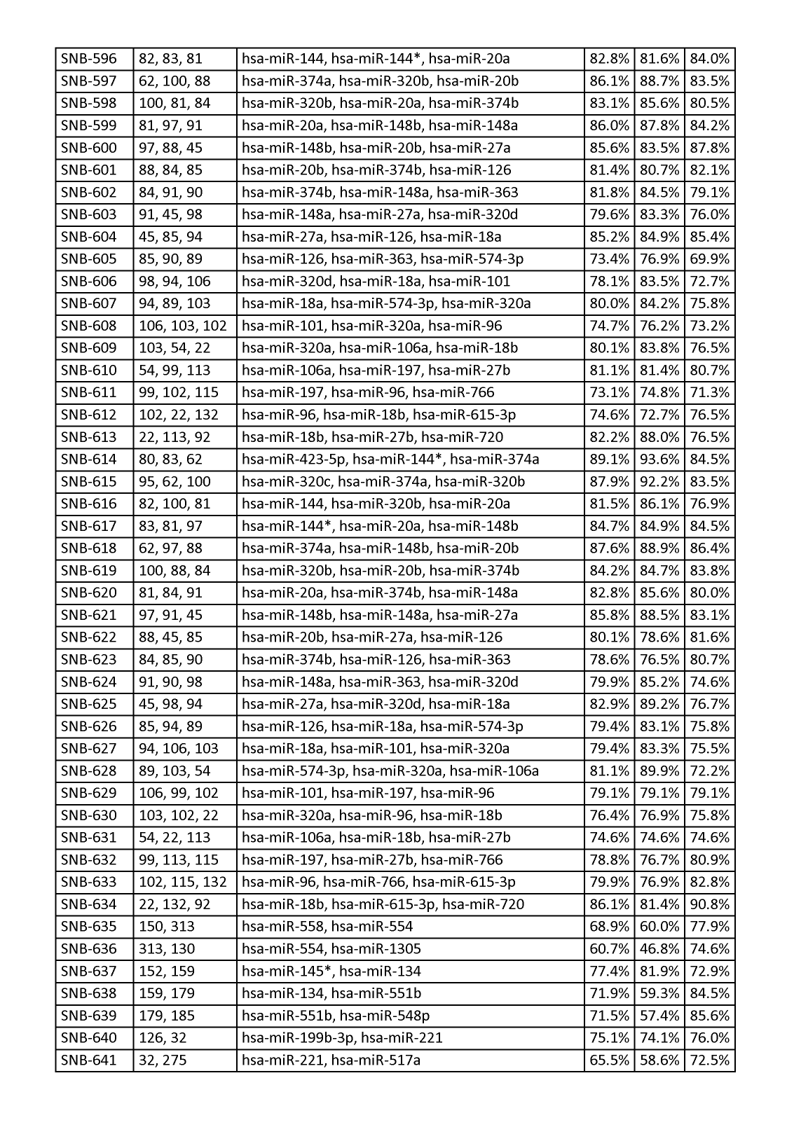

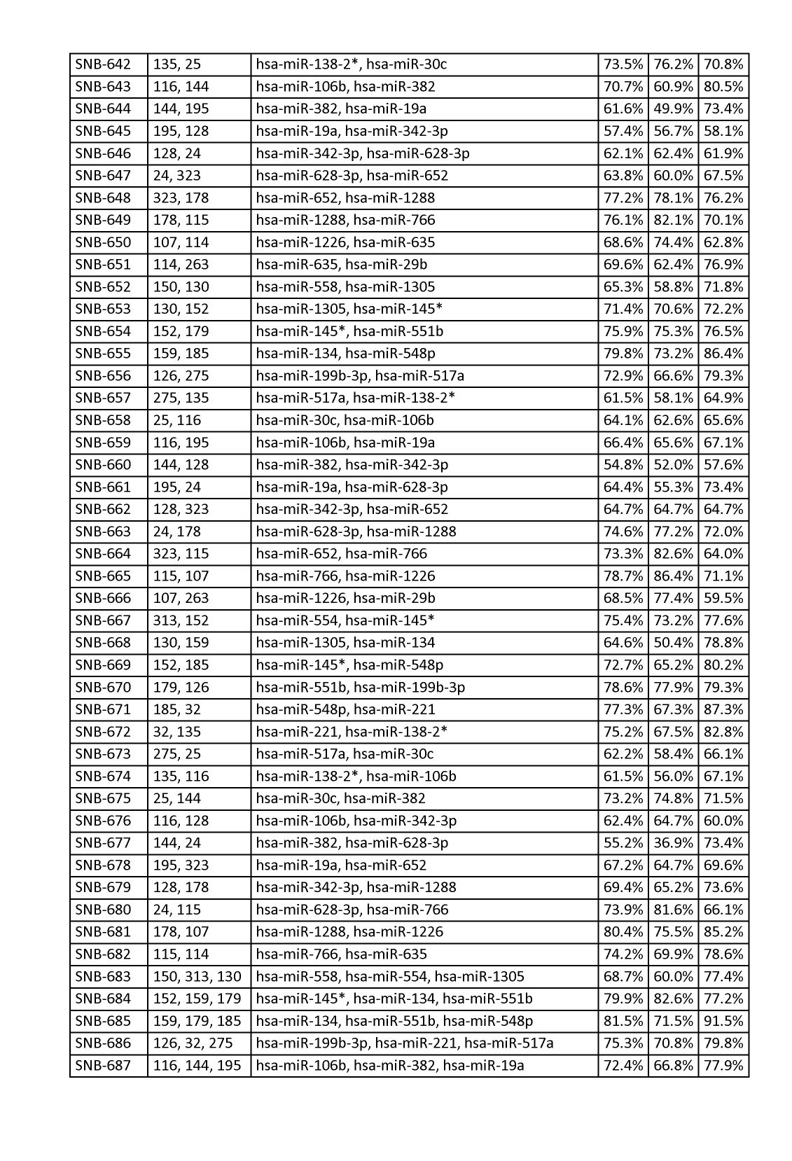

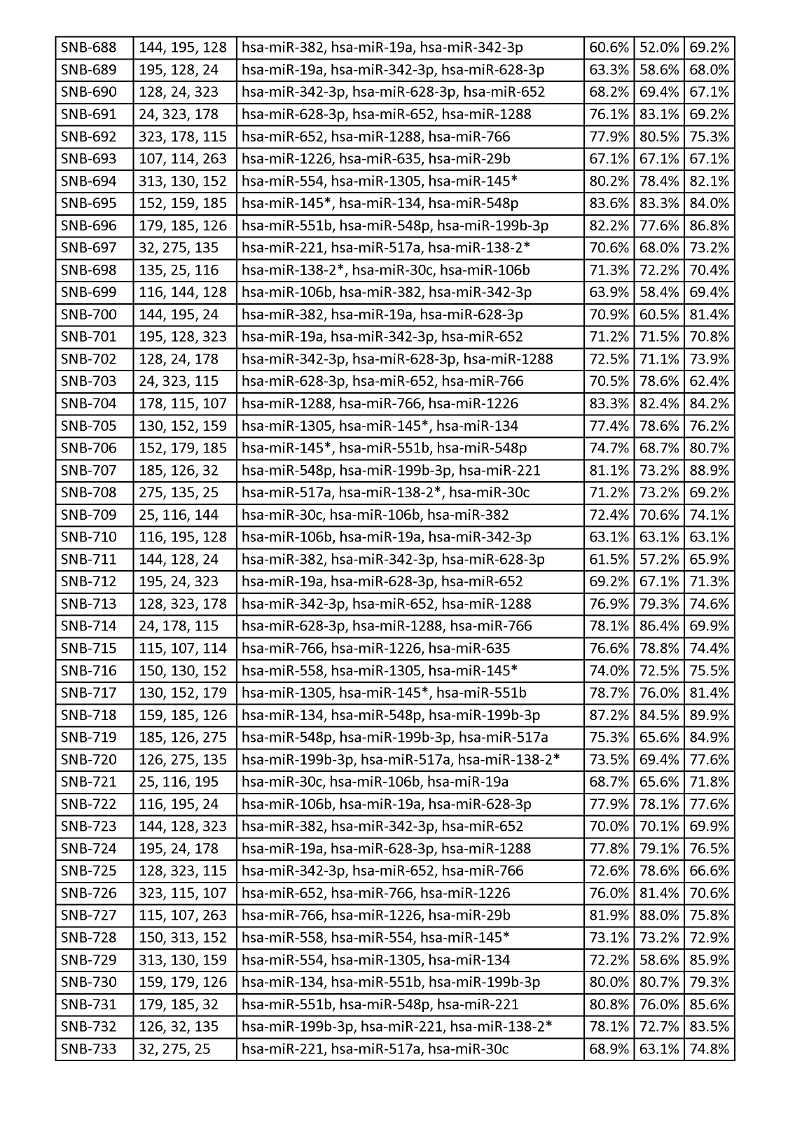

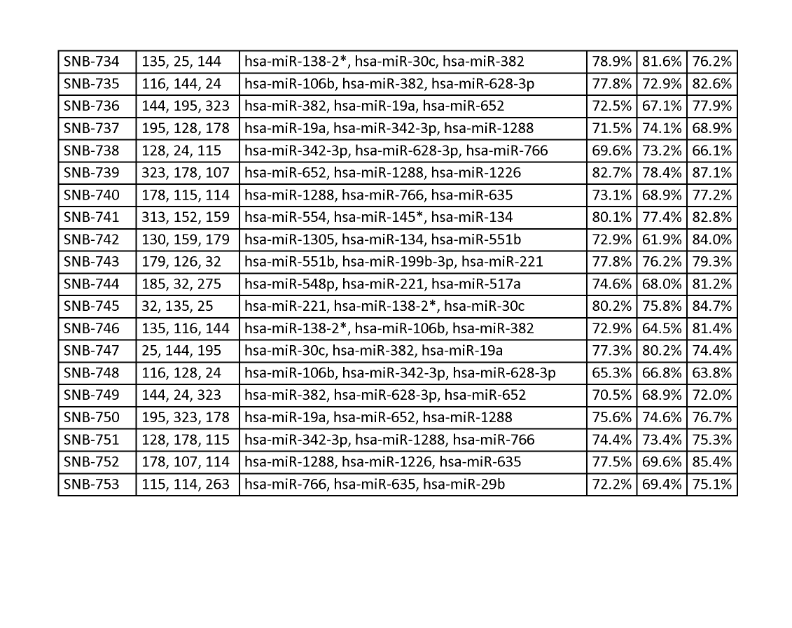

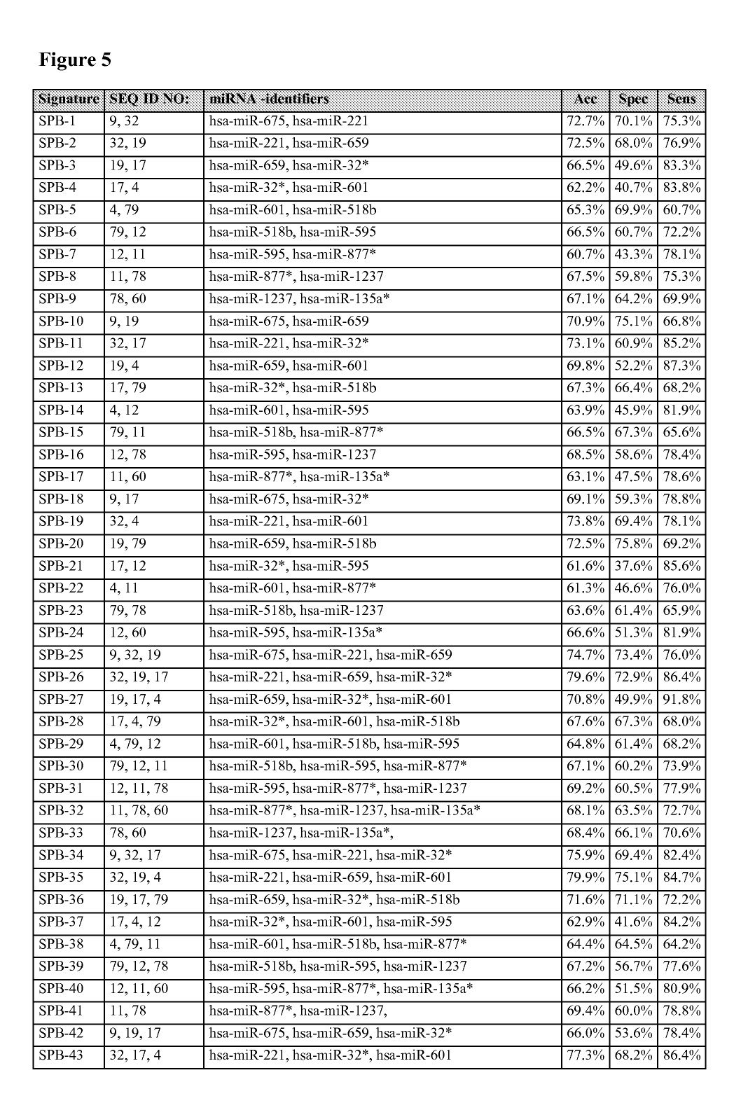

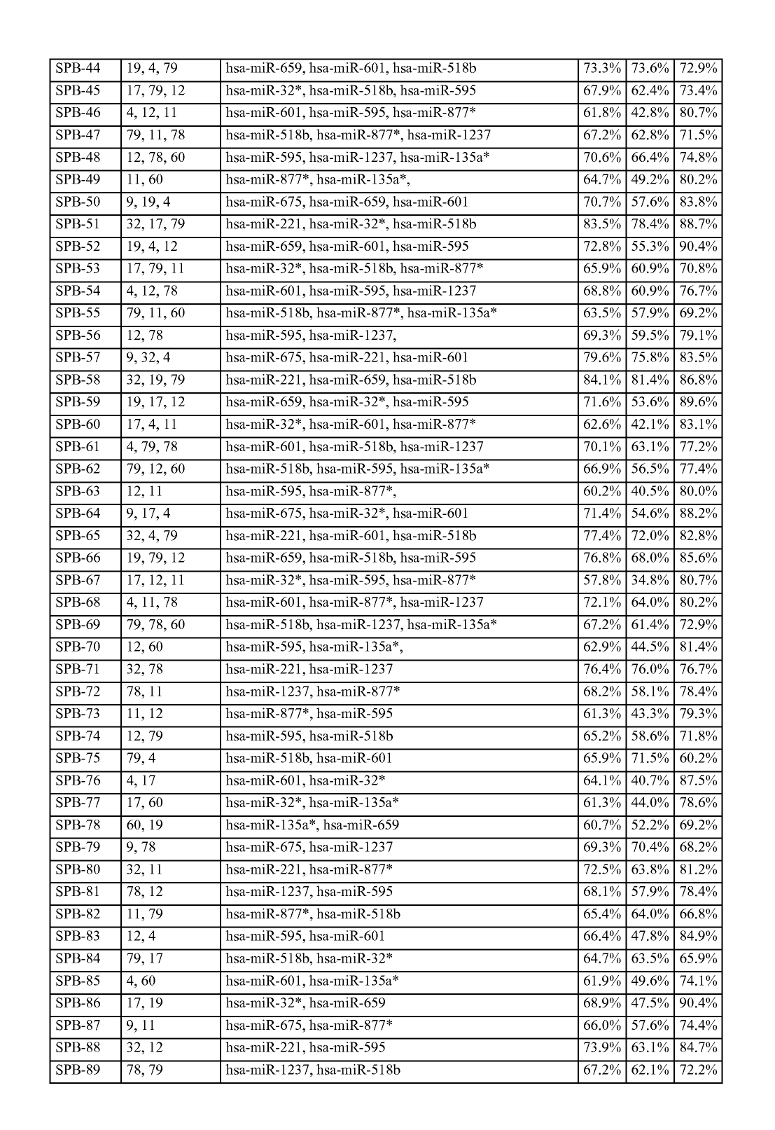

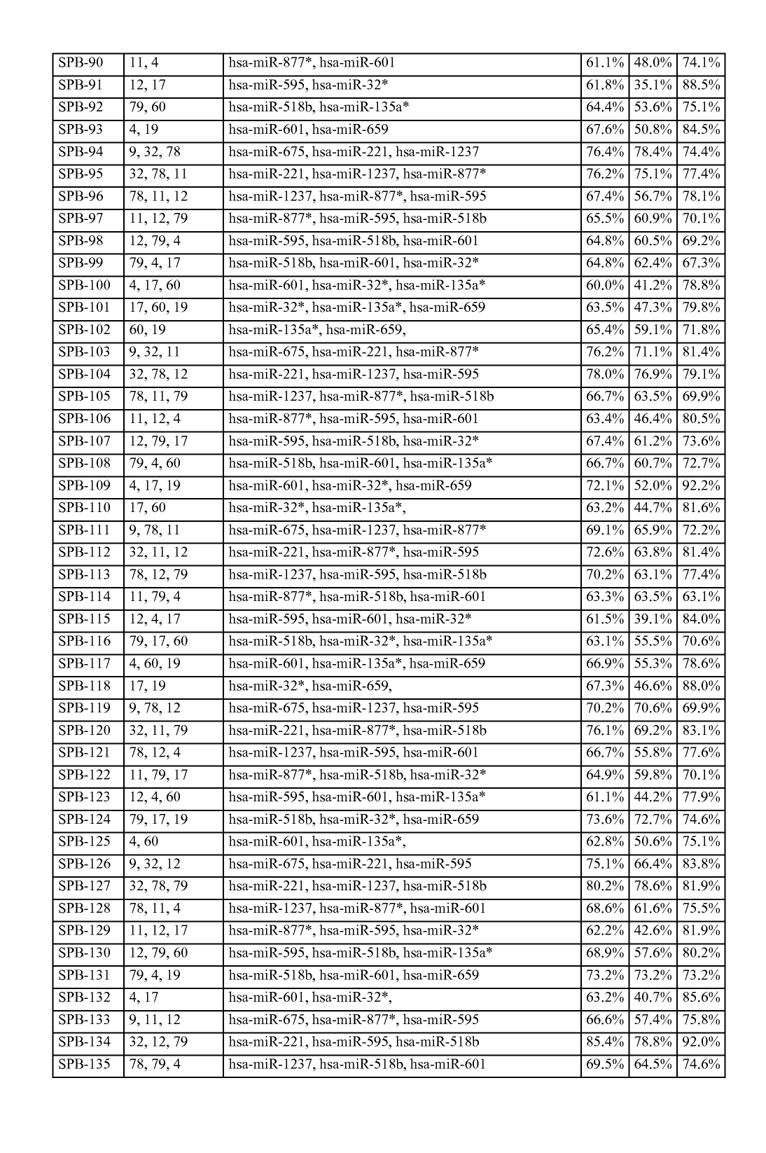

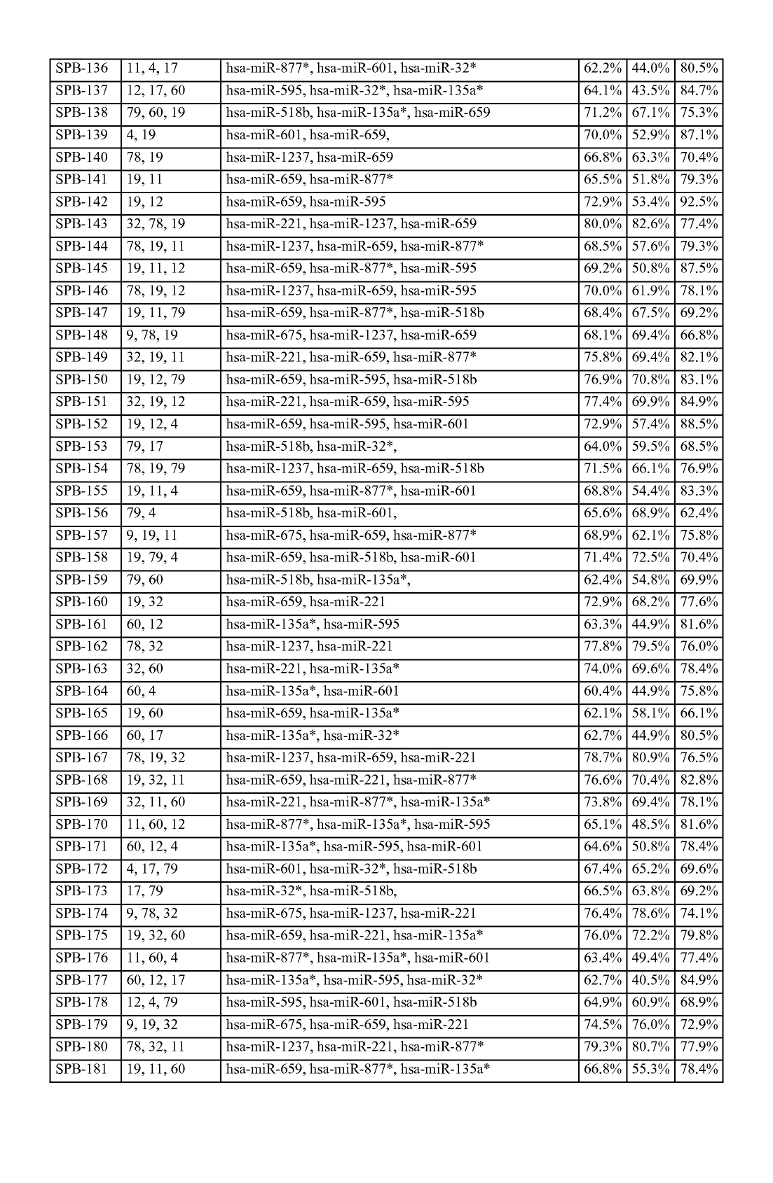

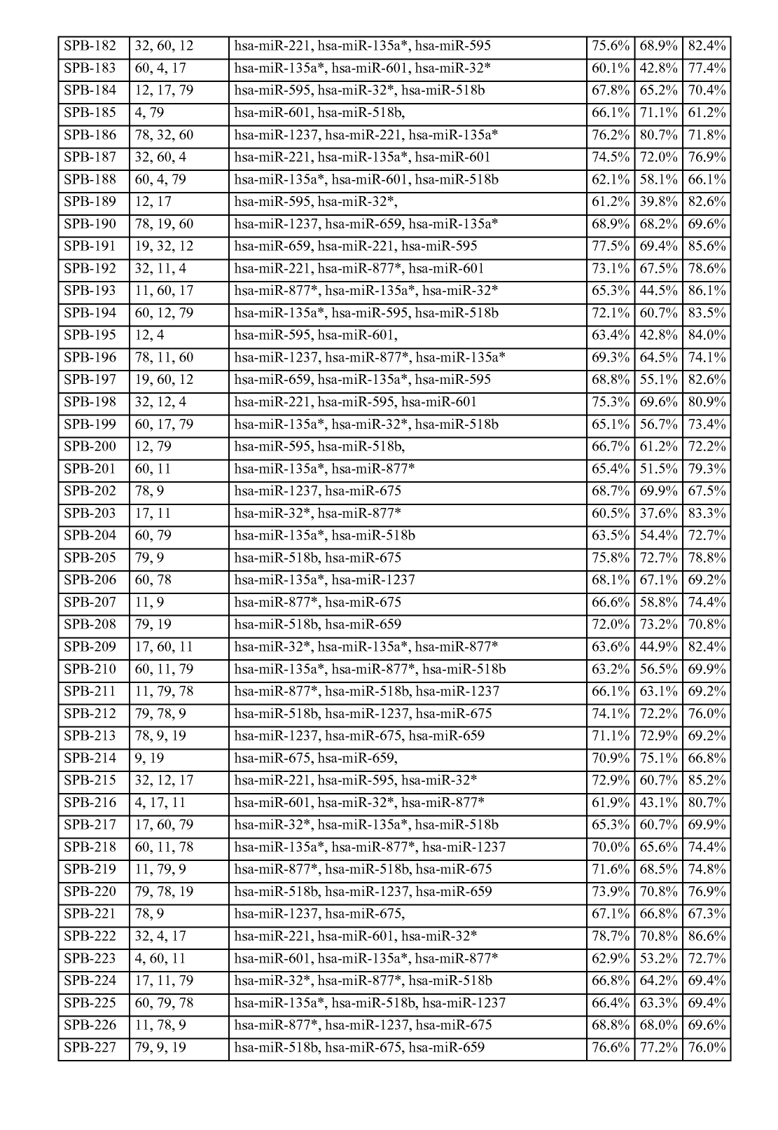

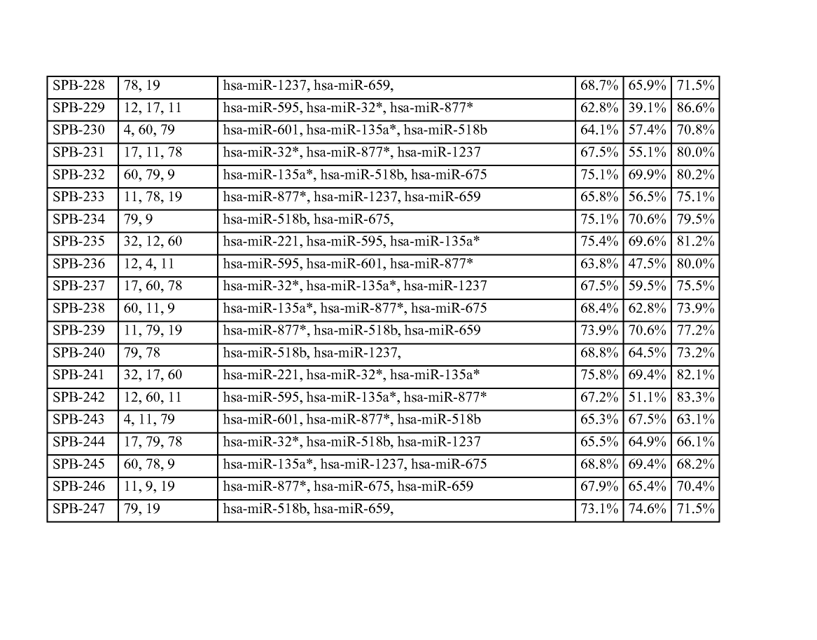

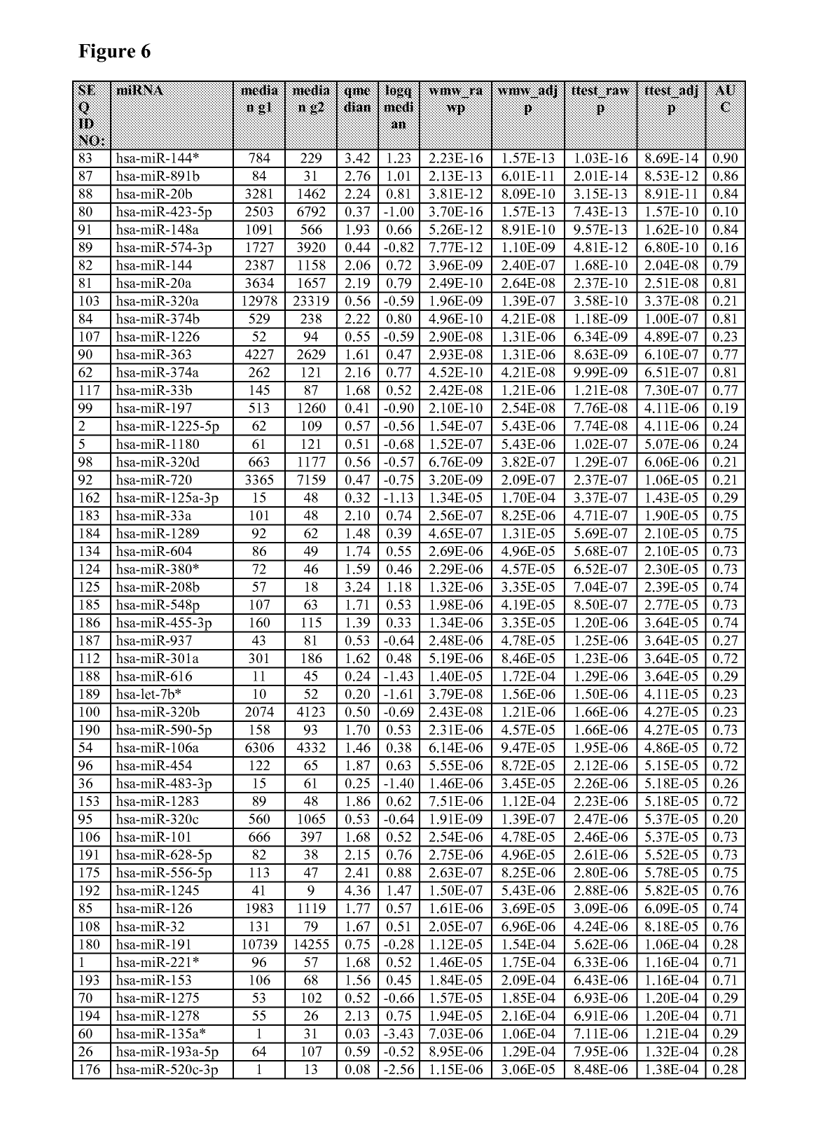

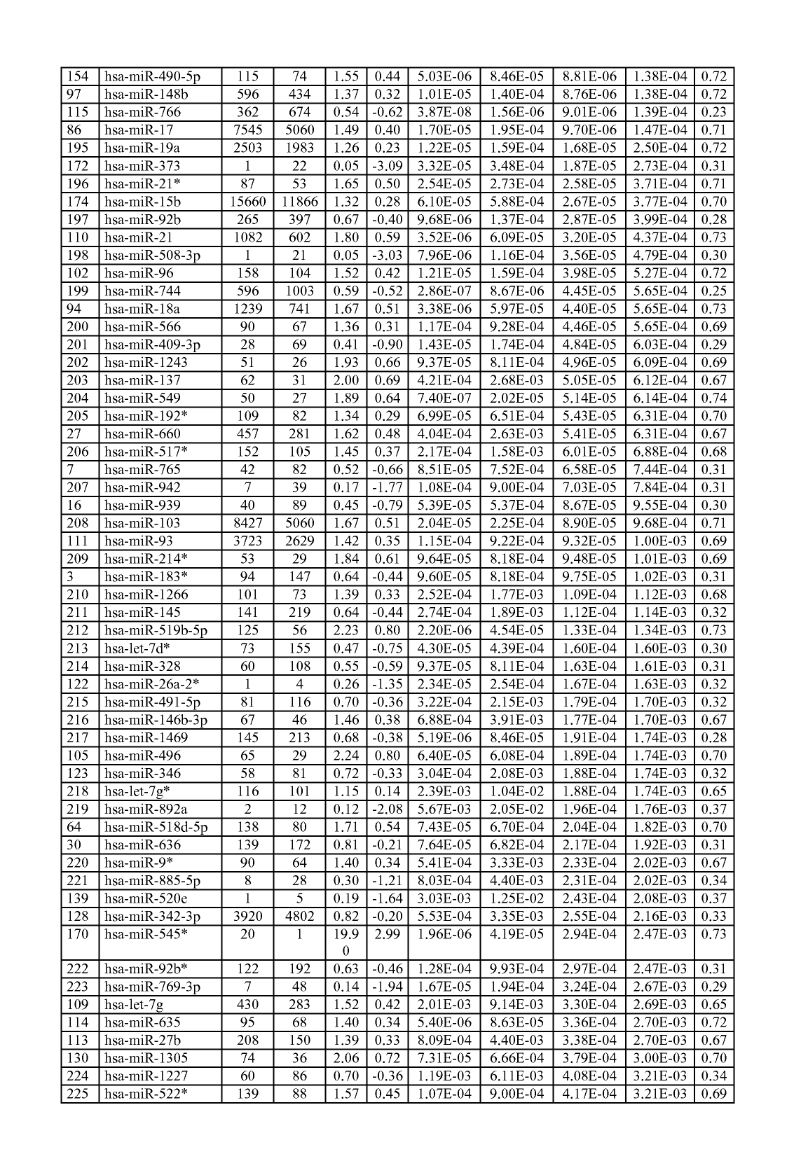

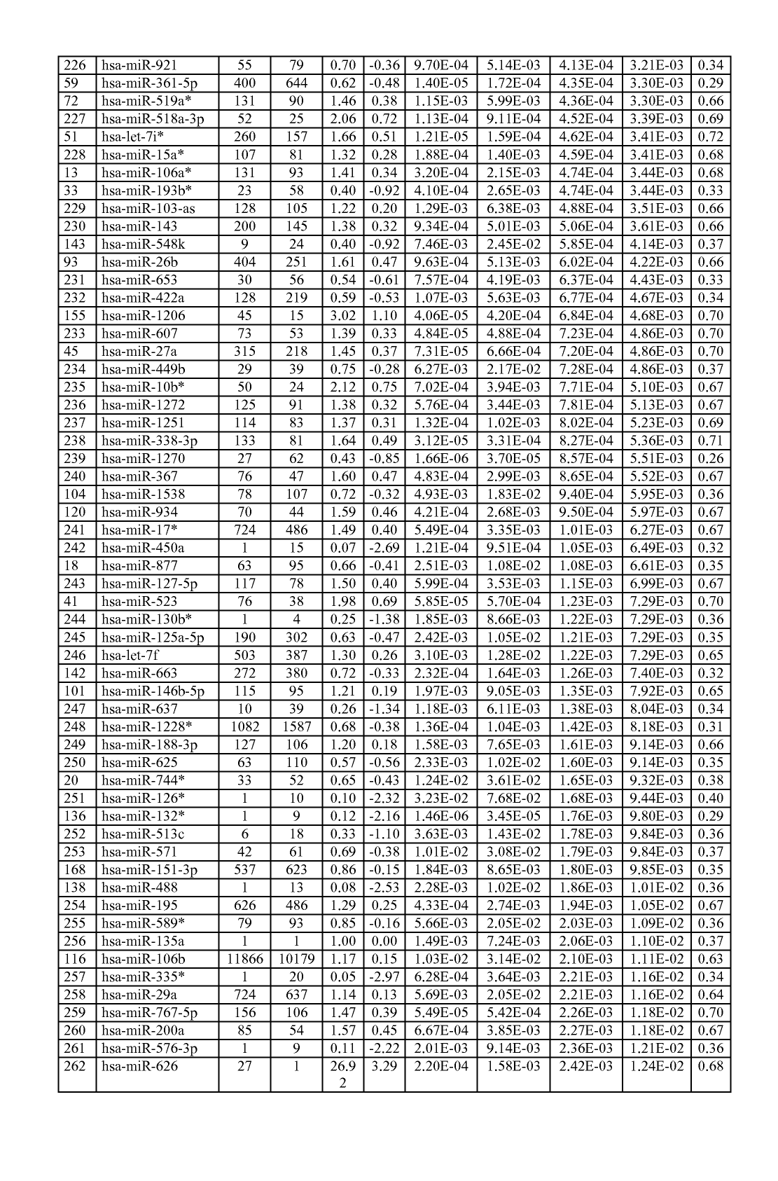

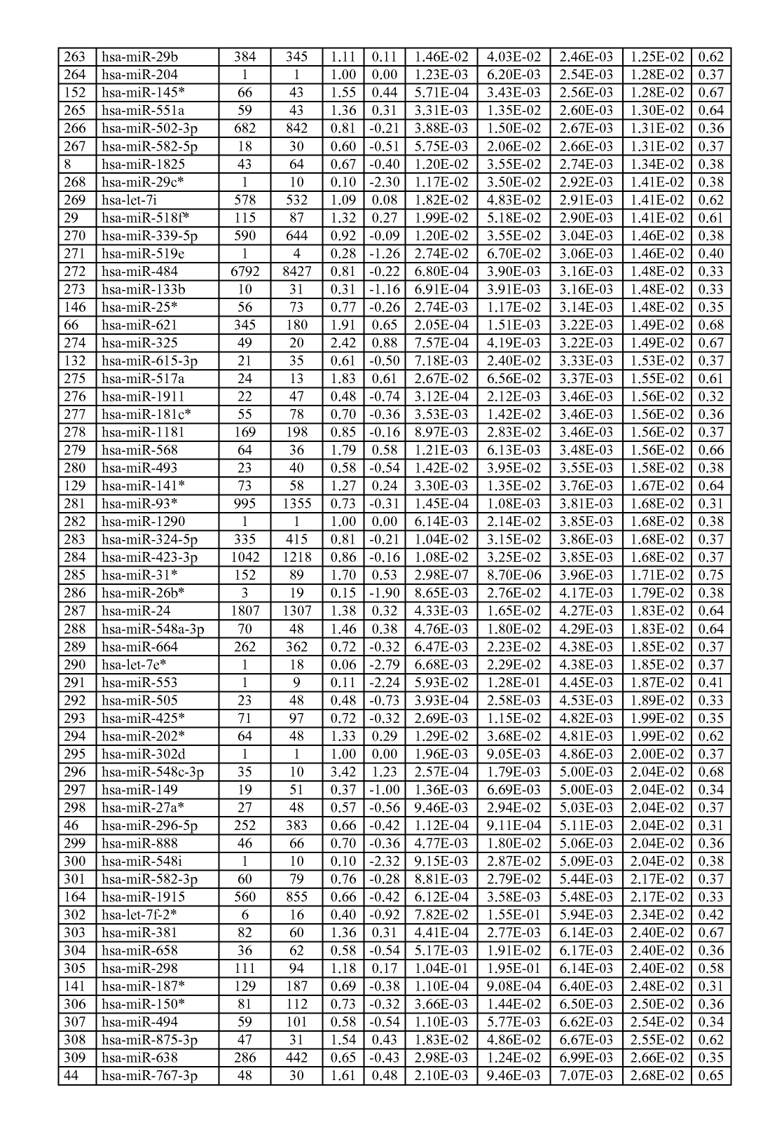

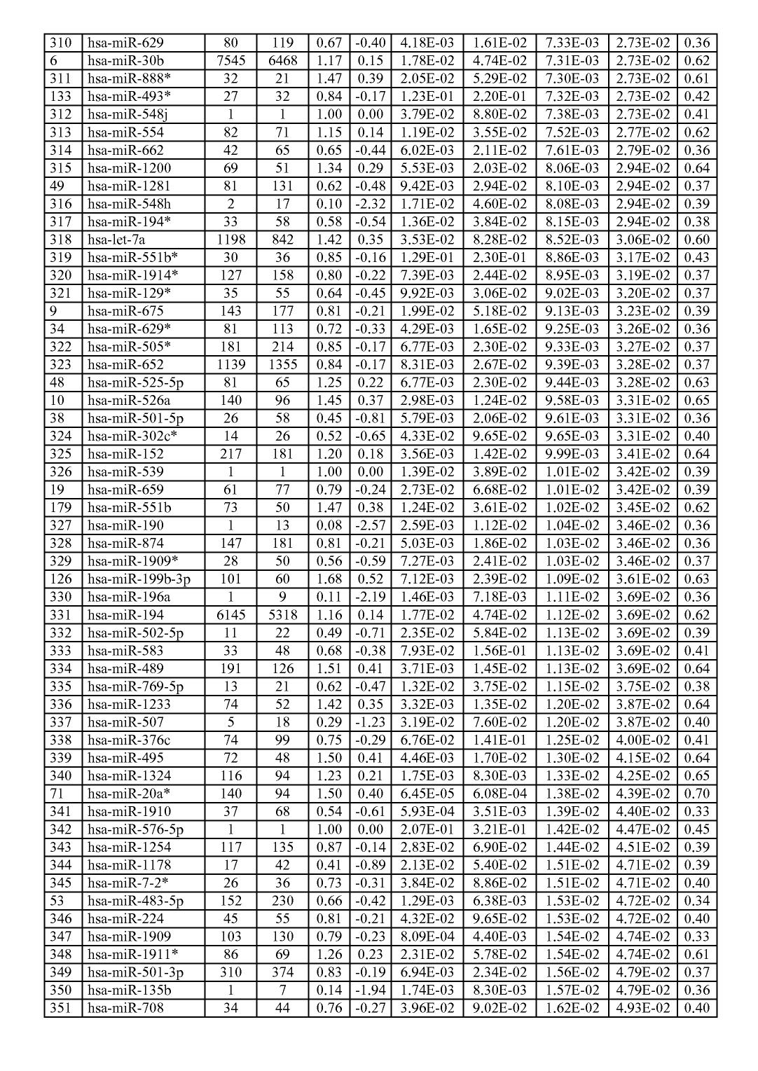

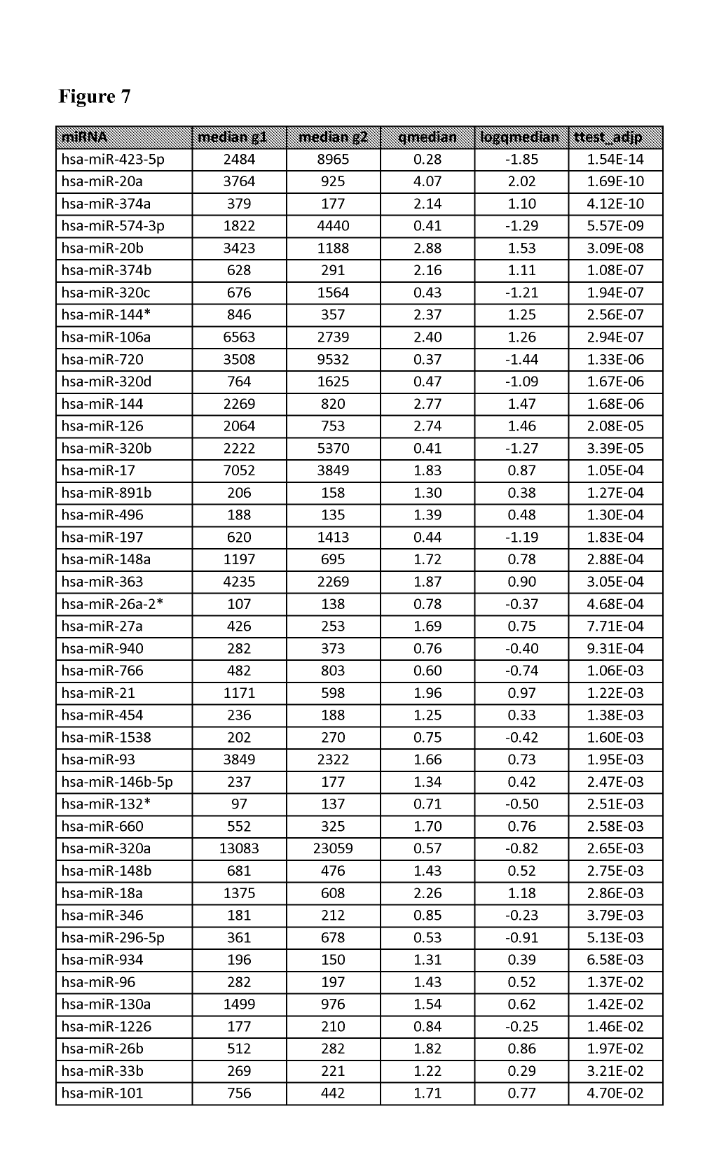

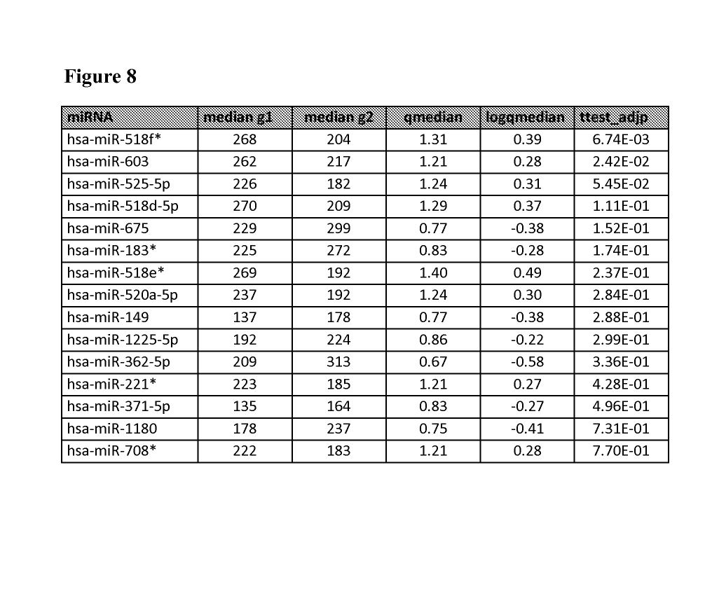

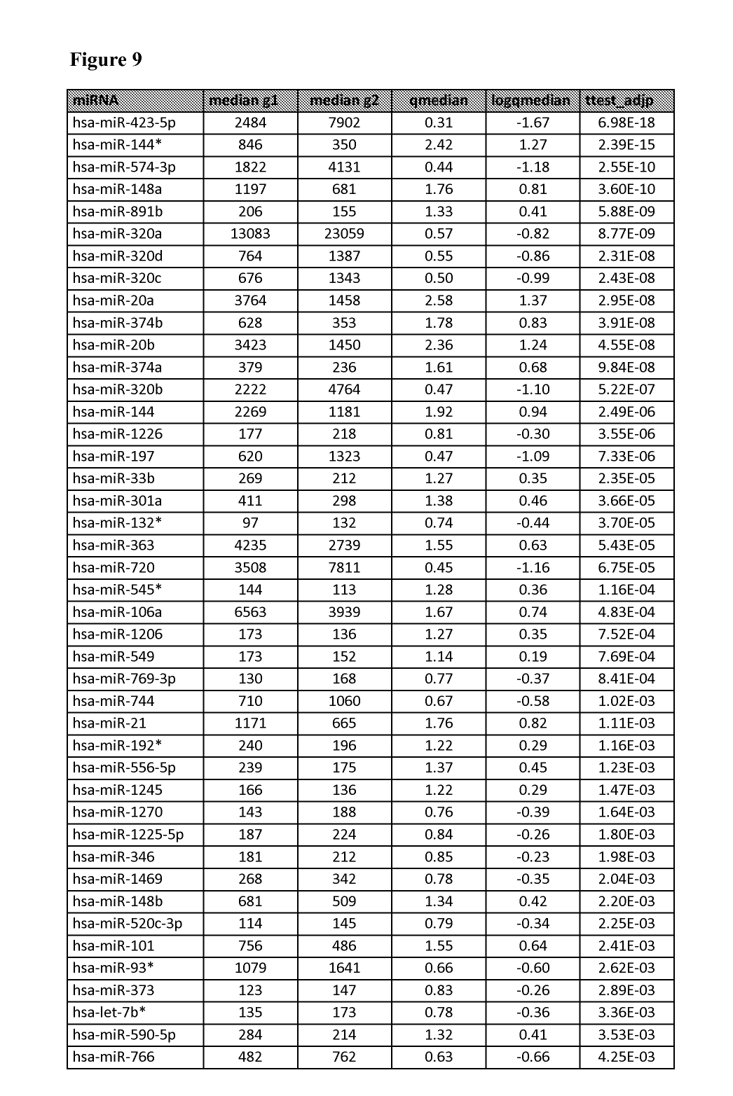

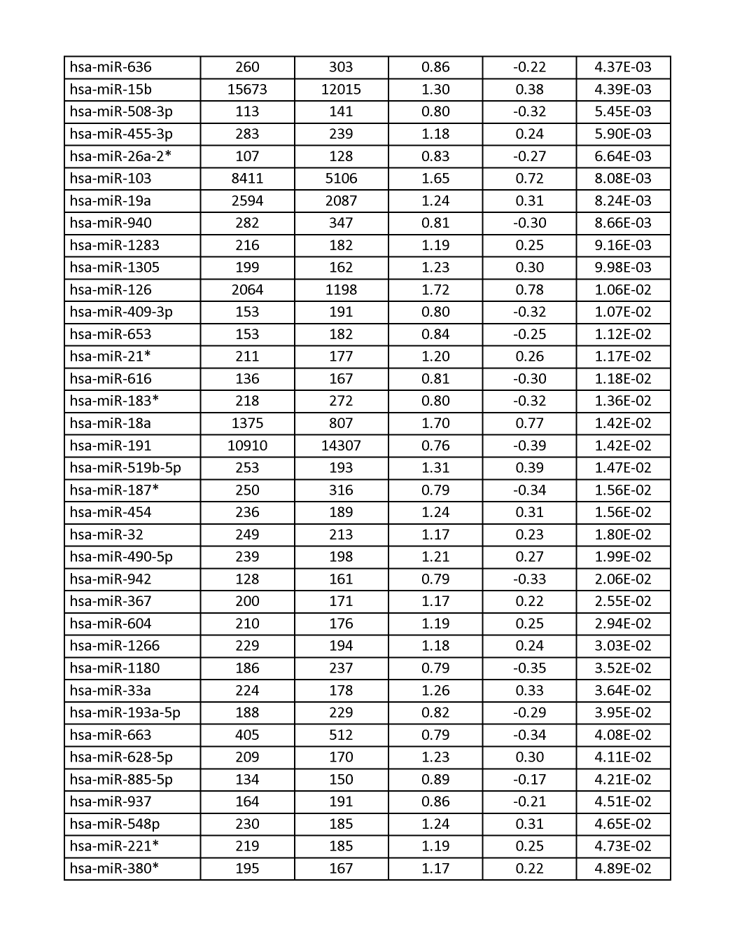

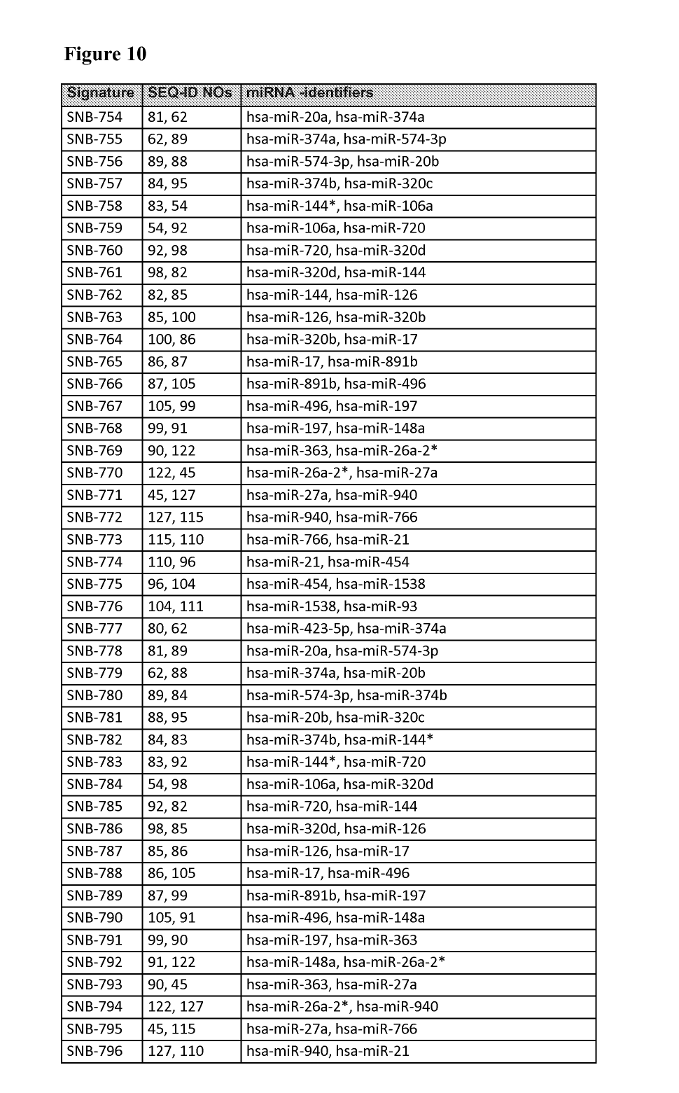

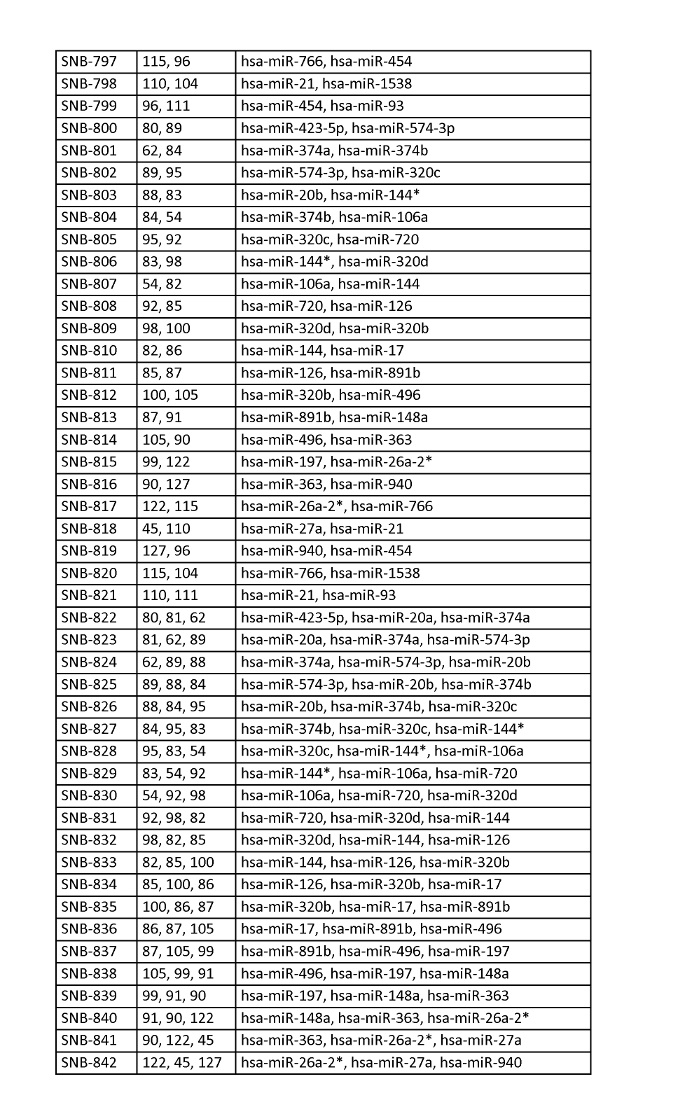

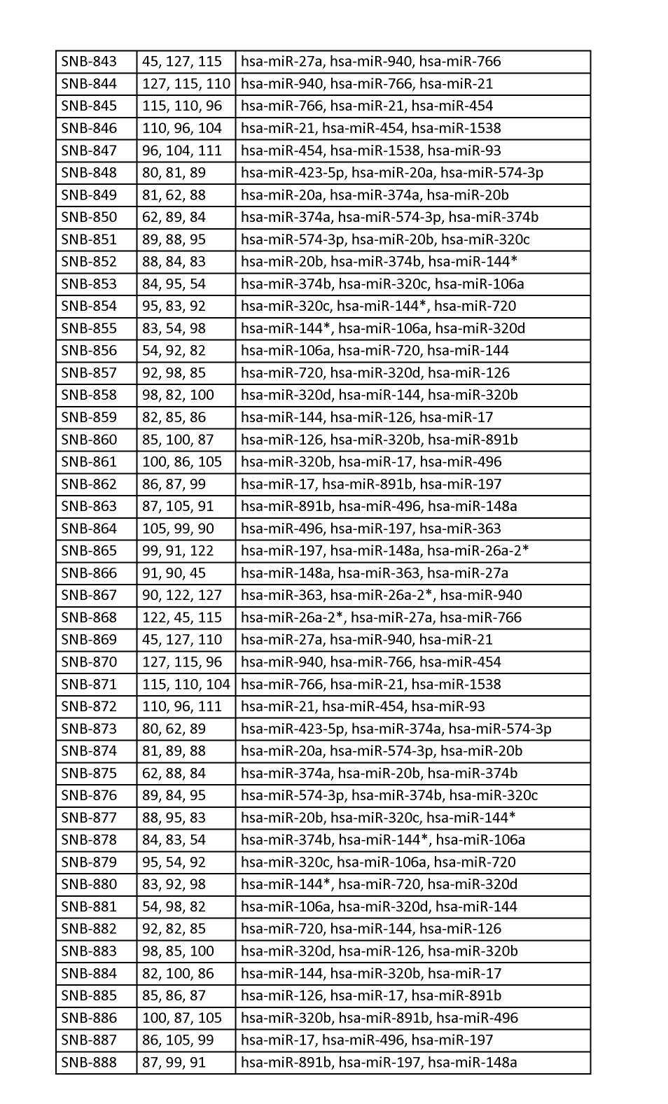

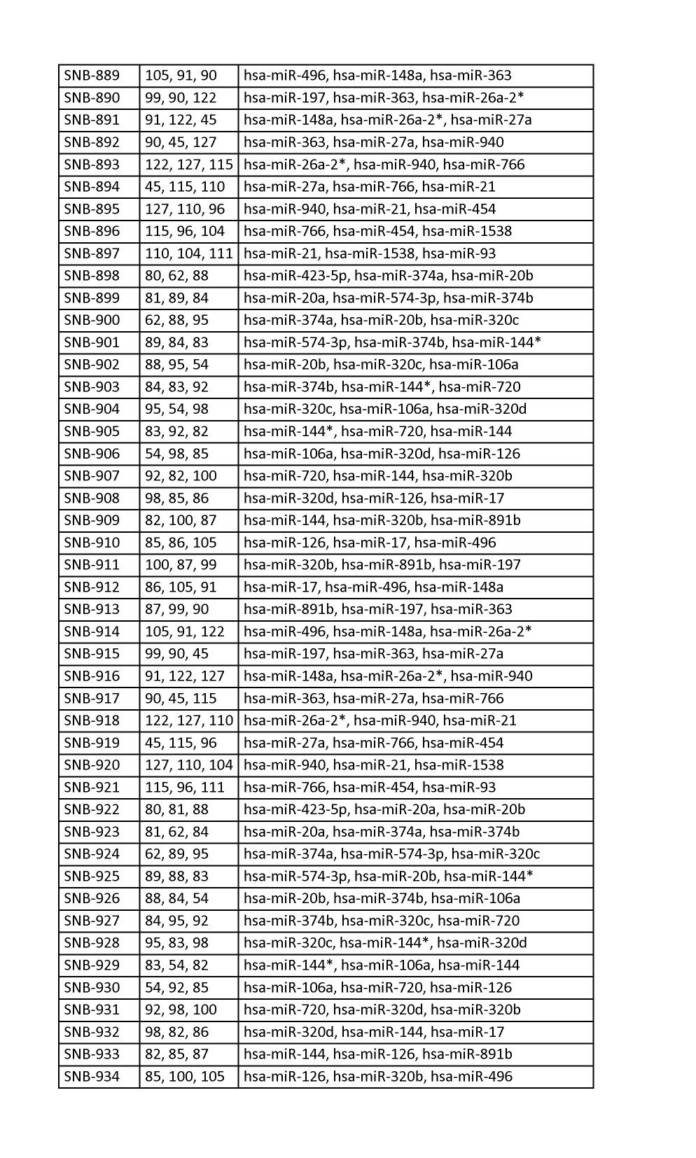

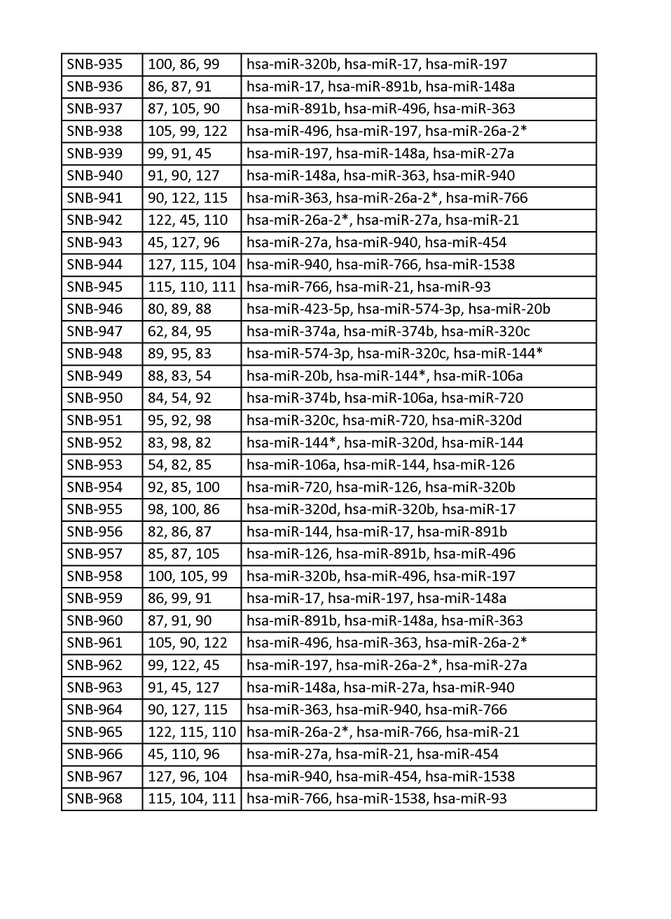

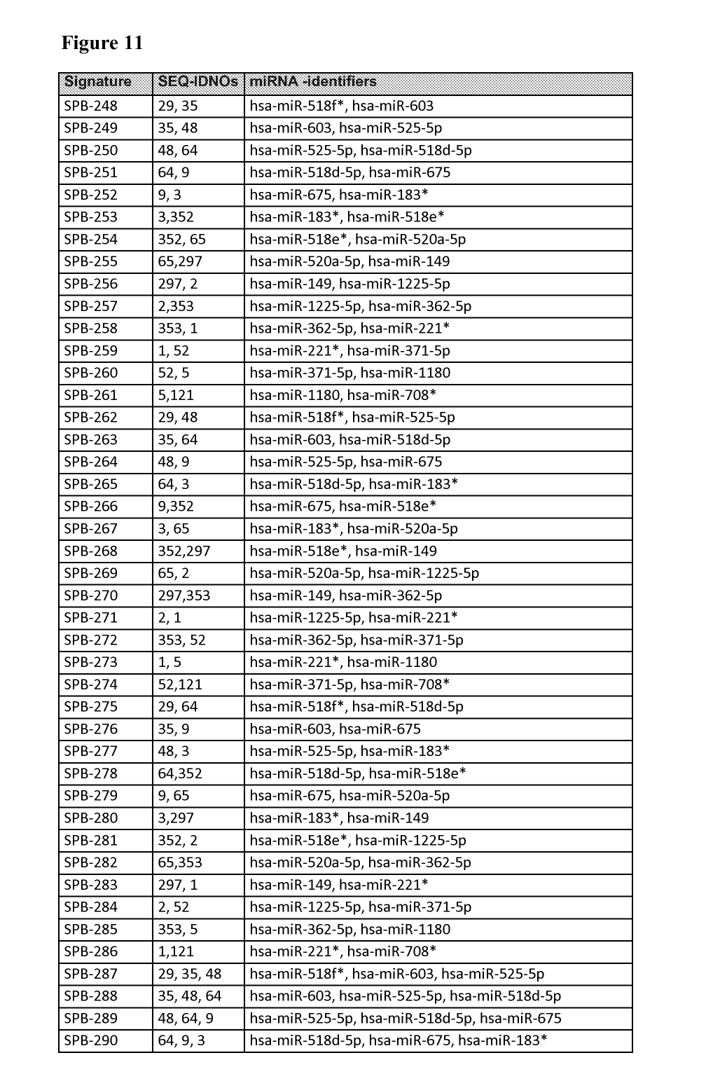

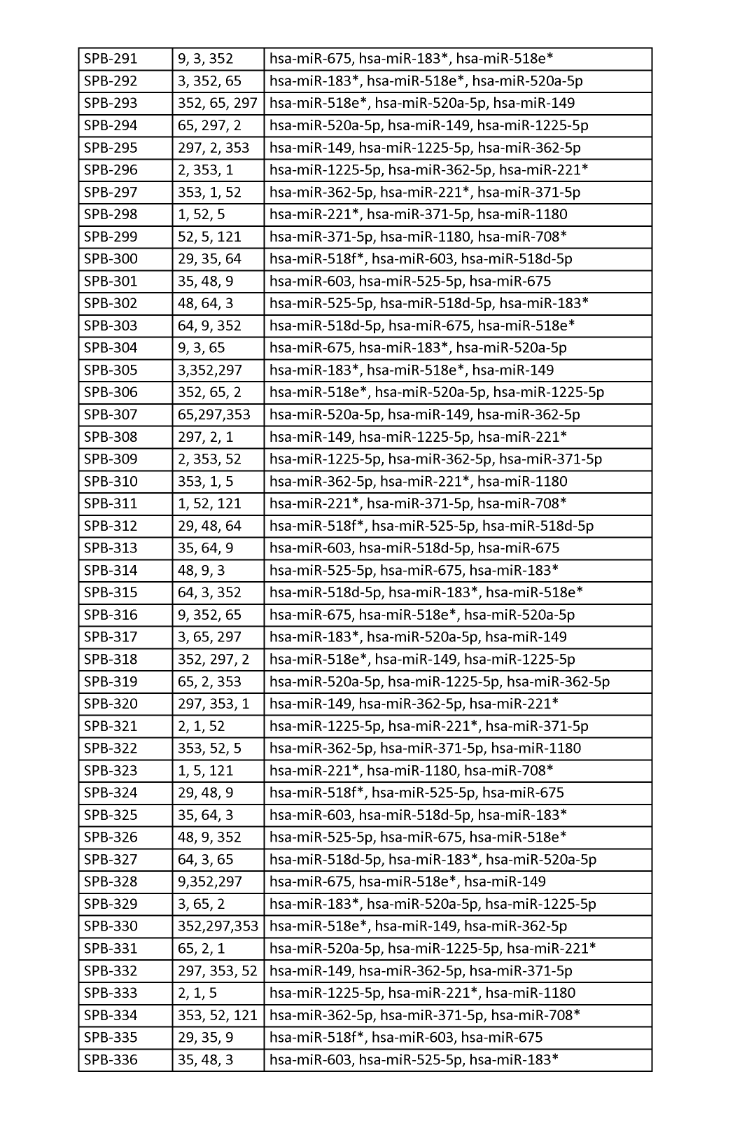

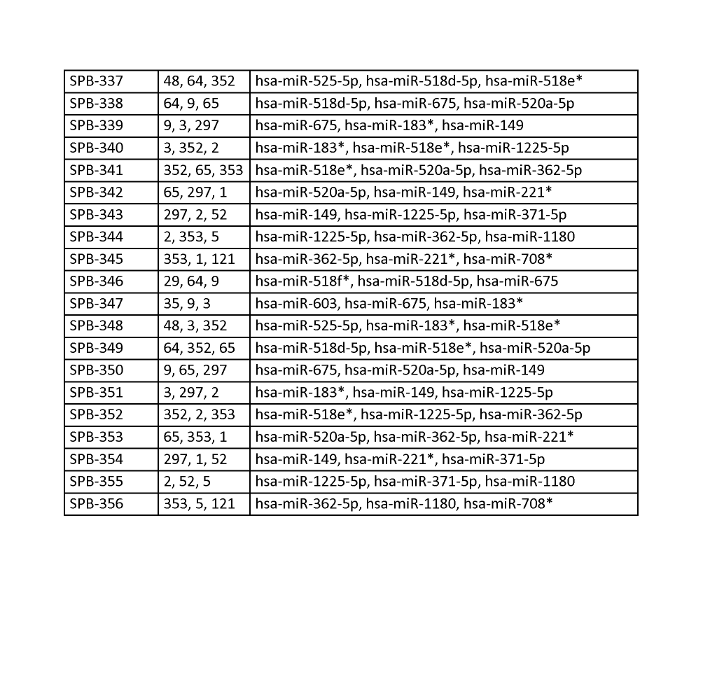

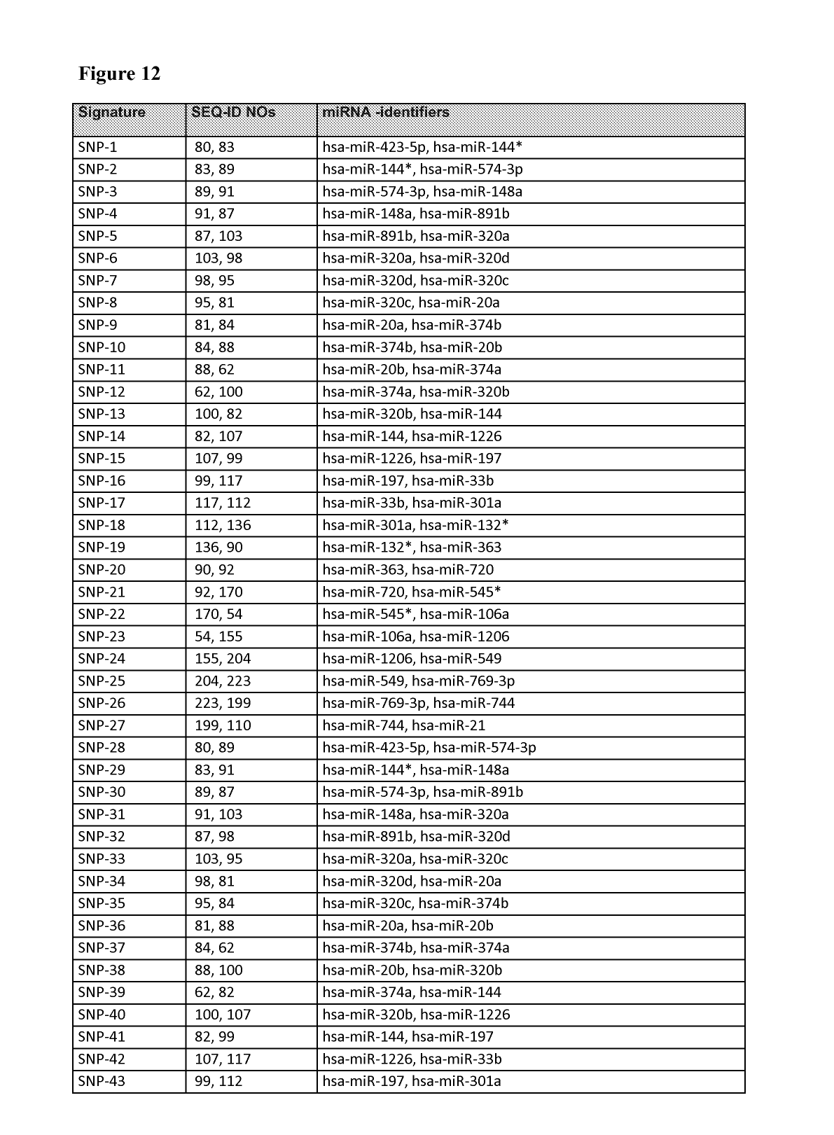

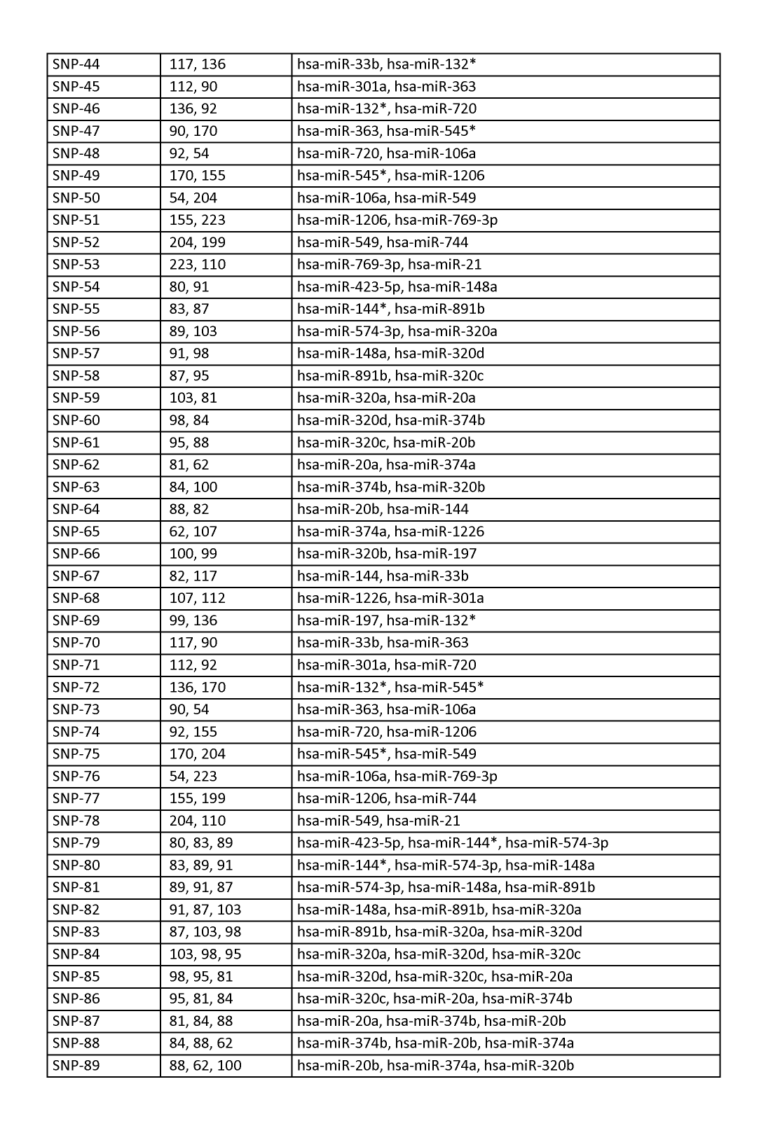

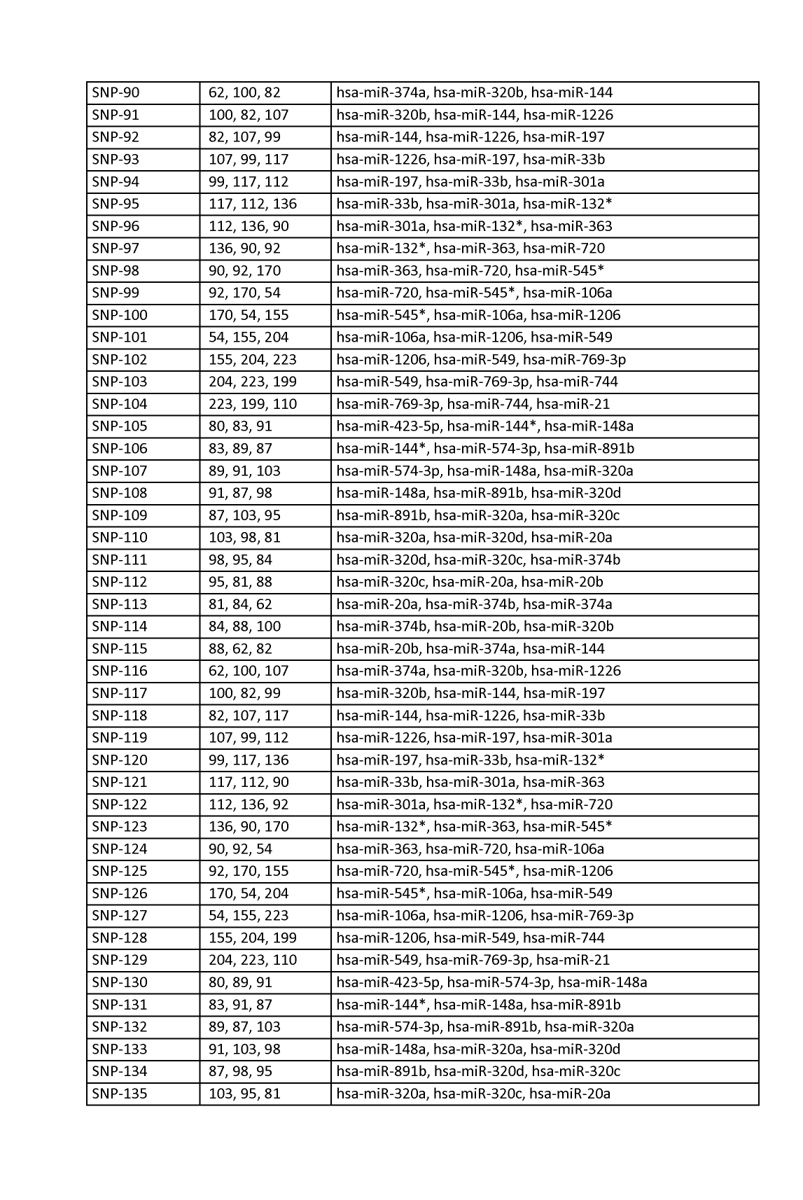

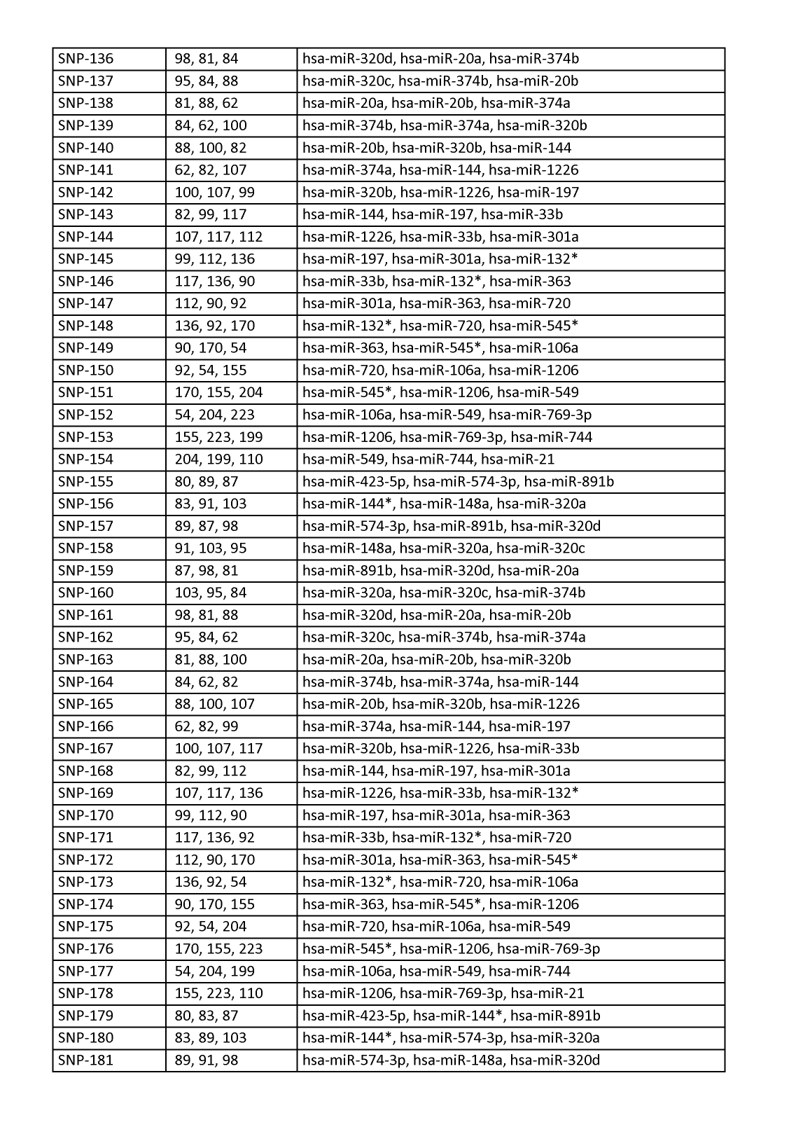

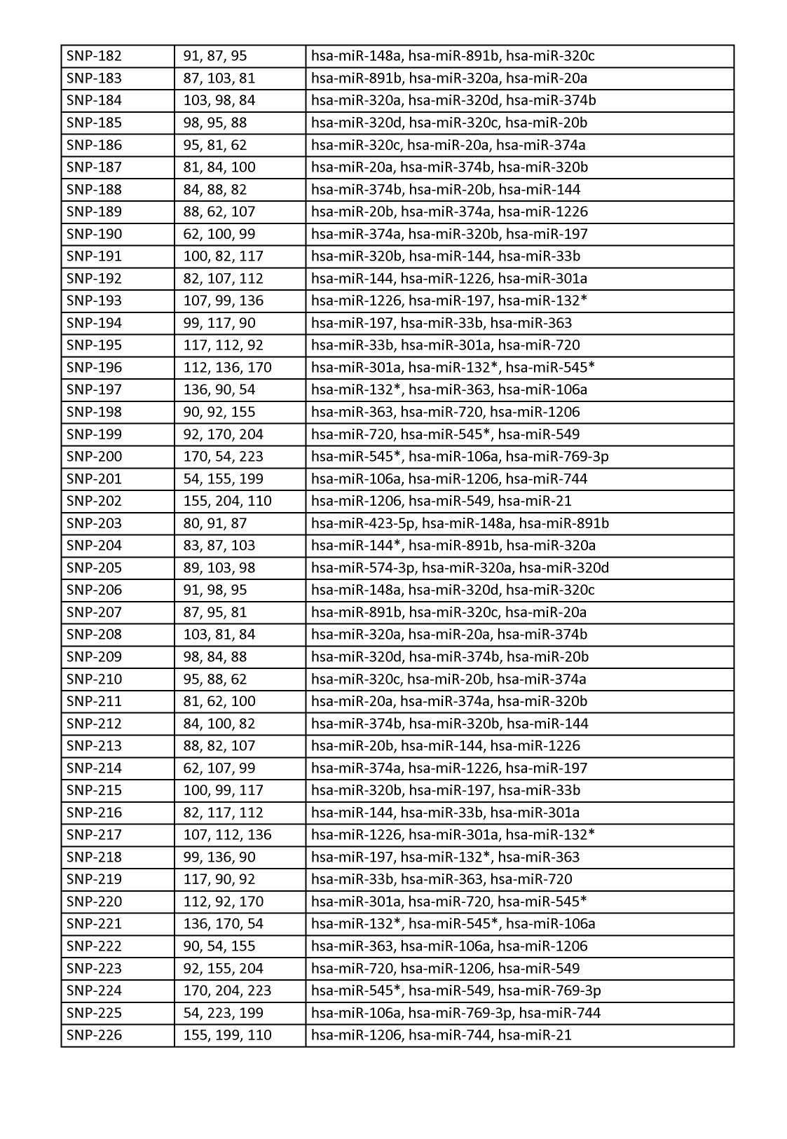

5. The method according to claim 1, wherein the set of miRNAs comprises miRNAs that are differentially regulated in blood samples from BPH patients as compared to healthy controls and wherein the miRNAs are selected from FIG. 2 or FIG. 7

6. The method according to claim 5, wherein the set of miRNAs comprises at least one set of miRNAs listed in FIG. 4 or FIG. 10

7. The method according to claim 1 wherein the expression profile is determined by nucleic acid hybridization, nucleic acid amplification, polymerase extension, sequencing, mass spectroscopy, flow cytometry or any combinations thereof.

8. A set of polynucleotides for detecting a set comprising at least two miRNAs for diagnosing BPH in a blood sample from a patient

9. The set of polynucleotides according to claim 8, wherein the blood sample is a blood cell sample or a leukocyte containing sample.

10. The set of polynucleotides according to claim 8, wherein the miRNAs are selected from the miRNAs that are differentially regulated in blood samples from BPH patients as compared to prostate cancer patients listed in FIG. 3 or FIG. 8

11. The set of polynucleotides according to claim 10, wherein the set of miRNAs comprises at least one set of miRNAs listed in FIG. 5 or FIG. 11

12. The set of polynucleotides according to claim 8, wherein the miRNAs are selected from the miRNAs that are differentially regulated in blood samples from BPH patients as compared to healthy controls listed in FIG. 2 or FIG. 7

13. The set of polynucleotides according to claim 12, wherein the set of miRNAs comprises at least one set of miRNAs listed in FIG. 4 or FIG. 10.

14. Means for diagnosing of BPH in a blood sample of a subject comprising:
(a) a set of at least two polynucleotides for detecting a set comprising at least two miRNAs wherein the miRNAs are selected from FIG. 2 or FIG. 3 or FIG. 7 or FIG. 8 or wherein the set of miRNAs comprises at least one set of miRNAs listed in FIG. 5 or FIG. 11 or FIG. 4 or FIG. 10, or
(b) a set of at least two primer pairs for detecting of at least a miRNA listed in FIG. 2 or FIG. 3 or FIG. 7 or FIG. 8

15. A kit for diagnosing BPH, comprising:
(a) means according to claim 14 for determining the miRNA expression profile of a RNA sample of a subject, and
(b) at least one reference

16. A set of miRNAs isolated from a blood sample from a subject for diagnosing of BPH, wherein the miRNAs are selected from FIG. 2 or FIG. 3 or FIG. 7 or FIG. 8 or wherein the set of miRNAs comprises at least one set of miRNAs listed in FIG. 5 or FIG. 11 or FIG. 4 or FIG. 10.

17. The set of miRNAs according to claim 16, wherein the blood sample is a blood cell sample or a leukocyte containing sample.

18. The set of miRNAs according to claim 16, wherein the set of miRNAs comprises miRNAs that are differentially regulated in blood samples from BPH patients as compared to prostate cancer patients and wherein the miRNAs are selected from FIG. 3 or FIG. 8

19. The set of miRNAs according to claim 18, wherein the set of miRNAs comprises at least one set of miRNAs listed in FIG. 5 or FIG. 11

20. The set of miRNAs according to claim 16, wherein the set of miRNAs comprises miRNAs that are differentially regulated in blood samples from BPH patients as compared to healthy controls and wherein the miRNAs are selected from FIG. 2 or FIG. 7

21. The set of miRNAs according to claim 20, wherein the set of miRNAs comprises at least one set of miRNAs listed in FIG. 4 or FIG. 10

22.-24. (canceled)


SEQUENCE LISTING (353 sequences)

SEQ ID NO: 1 - RNA, 22 nt, Homo sapiens
accuggcauacaauguagauuu

SEQ ID NO: 2 - RNA, 22 nt, Homo sapiens
guggguacggcccagugggggg

SEQ ID NO: 3 - RNA, 22 nt, Homo sapiens
gugaauuaccgaagggccauaa

SEQ ID NO: 4 - RNA, 22 nt, Homo sapiens
uggucuaggauuguuggaggag

SEQ ID NO: 5 - RNA, 22 nt, Homo sapiens
uuuccggcucgcgugggugugu

SEQ ID NO: 6 - RNA, 22 nt, Homo sapiens
uguaaacauccuacacucagcu

SEQ ID NO: 7 - RNA, 21 nt, Homo sapiens
uggaggagaaggaaggugaug

SEQ ID NO: 8 - RNA, 18 nt, Homo sapiens
uccagugcccuccucucc

SEQ ID NO: 9 - RNA, 23 nt, Homo sapiens
uggugcggagagggcccacagug

SEQ ID NO: 10 - RNA, 22 nt, Homo sapiens
cucuagagggaagcacuuucug

SEQ ID NO: 11 - RNA, 21 nt, Homo sapiens
uccucuucucccuccucccag

SEQ ID NO: 12 - RNA, 21 nt, Homo sapiens
gaagugugccguggugugucu

SEQ ID NO: 13 - RNA, 22 nt, Homo sapiens
cugcaauguaagcacuucuuac

SEQ ID NO: 14 - RNA, 18 nt, Homo sapiens
cgggcguggugguggggg

SEQ ID NO: 15 - RNA, 19 nt, Homo sapiens
gaugaugcugcugaugcug

SEQ ID NO: 16 - RNA, 24 nt, Homo sapiens
uggggagcugaggcucugggggug

SEQ ID NO: 17 - RNA, 22 nt, Homo sapiens
caauuuagugugugugauauuu

SEQ ID NO: 18 - RNA, 20 nt, Homo sapiens
guagaggagauggcgcaggg

SEQ ID NO: 19 - RNA, 22 nt, Homo sapiens
cuugguucagggagggucccca

SEQ ID NO: 20 - RNA, 22 nt, Homo sapiens
cuguugccacuaaccucaaccu

SEQ ID NO: 21 - RNA, 22 nt, Homo sapiens
uucacauugugcuacugucugc

SEQ ID NO: 22 - RNA, 23 nt, Homo sapiens
uaaggugcaucuagugcaguuag

SEQ ID NO: 23 - RNA, 23 nt, Homo sapiens
ugagugugugugugugagugugu

SEQ ID NO: 24 - RNA, 21 nt, Homo sapiens
ucuaguaagaguggcagucga

SEQ ID NO: 25 - RNA, 23 nt, Homo sapiens
uguaaacauccuacacucucagc

SEQ ID NO: 26 - RNA, 22 nt, Homo sapiens
ugggucuuugcgggcgagauga

SEQ ID NO: 27 - RNA, 22 nt, Homo sapiens
uacccauugcauaucggaguug

SEQ ID NO: 28 - RNA, 20 nt, Homo sapiens
cggcucugggucugugggga

SEQ ID NO: 29 - RNA, 22 nt, Homo sapiens
cucuagagggaagcacuuucuc

SEQ ID NO: 30 - RNA, 23 nt, Homo sapiens
ugugcuugcucgucccgcccgca

SEQ ID NO: 31 - RNA, 23 nt, Homo sapiens
aacauucauugcugucggugggu

SEQ ID NO: 32 - RNA, 23 nt, Homo sapiens
agcuacauugucugcuggguuuc

SEQ ID NO: 33 - RNA, 22 nt, Homo sapiens
cgggguuuugagggcgagauga

SEQ ID NO: 34 - RNA, 22 nt, Homo sapiens
guucucccaacguaagcccagc

SEQ ID NO: 35 - RNA, 22 nt, Homo sapiens
cacacacugcaauuacuuuugc

SEQ ID NO: 36 - RNA, 21 nt, Homo sapiens
ucacuccucuccucccgucuu

SEQ ID NO: 37 - RNA, 22 nt, Homo sapiens
aacccguagauccgaucuugug

SEQ ID NO: 38 - RNA, 22 nt, Homo sapiens
aauccuuugucccugggugaga

SEQ ID NO: 39 - RNA, 23 nt, Homo sapiens
aucgcugcgguugcgagcgcugu

SEQ ID NO: 40 - RNA, 21 nt, Homo sapiens
ugcaggaccaagaugagcccu

SEQ ID NO: 41 - RNA, 23 nt, Homo sapiens
gaacgcgcuucccuauagagggu

SEQ ID NO: 42 - RNA, 23 nt, Homo sapiens
aucaacagacauuaauugggcgc

SEQ ID NO: 43 - RNA, 22 nt, Homo sapiens
gcuacuucacaacaccagggcc

SEQ ID NO: 44 - RNA, 23 nt, Homo sapiens
ucugcucauaccccaugguuucu

SEQ ID NO: 45 - RNA, 21 nt, Homo sapiens
uucacaguggcuaaguuccgc

SEQ ID NO: 46 - RNA, 21 nt, Homo sapiens
agggcccccccucaauccugu

SEQ ID NO: 47 - RNA, 23 nt, Homo sapiens
agcugguguugugaaucaggccg

SEQ ID NO: 48 - RNA, 21 nt, Homo sapiens
cuccagagggaugcacuuucu

SEQ ID NO: 49 - RNA, 17 nt, Homo sapiens
ucgccuccuccucuccc

SEQ ID NO: 50 - RNA, 22 nt, Homo sapiens
acucaaaacccuucagugacuu

SEQ ID NO: 51 - RNA, 22 nt, Homo sapiens
cugcgcaagcuacugccuugcu

SEQ ID NO: 52 - RNA, 20 nt, Homo sapiens
acucaaacugugggggcacu

SEQ ID NO: 53 - RNA, 22 nt, Homo sapiens
aagacgggaggaaagaagggag

SEQ ID NO: 54 - RNA, 23 nt, Homo sapiens
aaaagugcuuacagugcagguag

SEQ ID NO: 55 - RNA, 21 nt, Homo sapiens
caaaacgugaggcgcugcuau

SEQ ID NO: 56 - RNA, 21 nt, Homo sapiens
cuucuugugcucuaggauugu

SEQ ID NO: 57 - RNA, 22 nt, Homo sapiens
caacaaaucacagucugccaua

SEQ ID NO: 58 - RNA, 25 nt, Homo sapiens
aggggugguguugggacagcuccgu

SEQ ID NO: 59 - RNA, 22 nt, Homo sapiens
uuaucagaaucuccagggguac

SEQ ID NO: 60 - RNA, 22 nt, Homo sapiens
uauagggauuggagccguggcg

SEQ ID NO: 61 - RNA, 21 nt, Homo sapiens
ucuuguguucucuagaucagu

SEQ ID NO: 62 - RNA, 22 nt, Homo sapiens
uuauaauacaaccugauaagug

SEQ ID NO: 63 - RNA, 27 nt, Homo sapiens
cacuguaggugauggugagagugggca

SEQ ID NO: 64 - RNA, 22 nt, Homo sapiens
cucuagagggaagcacuuucug

SEQ ID NO: 65 - RNA, 21 nt, Homo sapiens
cuccagagggaaguacuuucu

SEQ ID NO: 66 - RNA, 21 nt, Homo sapiens
ggcuagcaacagcgcuuaccu

SEQ ID NO: 67 - RNA, 22 nt, Homo sapiens
ccaaaacugcaguuacuuuugc

SEQ ID NO: 68 - RNA, 21 nt, Homo sapiens
cuuuuugcggucugggcuugc

SEQ ID NO: 69 - RNA, 20 nt, Homo sapiens
ucugcaggguuugcuuugag

SEQ ID NO: 70 - RNA, 17 nt, Homo sapiens
gugggggagaggcuguc

SEQ ID NO: 71 - RNA, 22 nt, Homo sapiens
acugcauuaugagcacuuaaag

SEQ ID NO: 72 - RNA, 22 nt, Homo sapiens
cucuagagggaagcgcuuucug

SEQ ID NO: 73 - RNA, 22 nt, Homo sapiens
cggguggaucacgaugcaauuu

SEQ ID NO: 74 - RNA, 21 nt, Homo sapiens
agagaagaagaucagccugca

SEQ ID NO: 75 - RNA, 21 nt, Homo sapiens
guggcugcacucacuuccuuc

SEQ ID NO: 76 - RNA, 20 nt, Homo sapiens
ccucugggcccuuccuccag

SEQ ID NO: 77 - RNA, 21 nt, Homo sapiens
aguuaaugaauccuggaaagu

SEQ ID NO: 78 - RNA, 21 nt, Homo sapiens
uccuucugcuccgucccccag

SEQ ID NO: 79 - RNA, 22 nt, Homo sapiens
caaagcgcuccccuuuagaggu

SEQ ID NO: 80 - RNA, 23 nt, Homo sapiens
ugaggggcagagagcgagacuuu

SEQ ID NO: 81 - RNA, 23 nt, Homo sapiens
uaaagugcuuauagugcagguag

SEQ ID NO: 82 - RNA, 20 nt, Homo sapiens
uacaguauagaugauguacu

SEQ ID NO: 83 - RNA, 22 nt, Homo sapiens
ggauaucaucauauacuguaag

SEQ ID NO: 84 - RNA, 22 nt, Homo sapiens
auauaauacaaccugcuaagug

SEQ ID NO: 85 - RNA, 22 nt, Homo sapiens
ucguaccgugaguaauaaugcg

SEQ ID NO: 86 - RNA, 23 nt, Homo sapiens
caaagugcuuacagugcagguag

SEQ ID NO: 87 - RNA, 22 nt, Homo sapiens
ugcaacuuaccugagucauuga

SEQ ID NO: 88 - RNA, 23 nt, Homo sapiens
caaagugcucauagugcagguag

SEQ ID NO: 89 - RNA, 22 nt, Homo sapiens
cacgcucaugcacacacccaca

SEQ ID NO: 90 - RNA, 22 nt, Homo sapiens
aauugcacgguauccaucugua

SEQ ID NO: 91 - RNA, 22 nt, Homo sapiens
ucagugcacuacagaacuuugu

SEQ ID NO: 92 - RNA, 17 nt, Homo sapiens
ucucgcuggggccucca

SEQ ID NO: 93 - RNA, 21 nt, Homo sapiens
uucaaguaauucaggauaggu

SEQ ID NO: 94 - RNA, 23 nt, Homo sapiens
uaaggugcaucuagugcagauag

SEQ ID NO: 95 - RNA, 20 nt, Homo sapiens
aaaagcuggguugagagggu

SEQ ID NO: 96 - RNA, 23 nt, Homo sapiens
uagugcaauauugcuuauagggu

SEQ ID NO: 97 - RNA, 22 nt, Homo sapiens
ucagugcaucacagaacuuugu

SEQ ID NO: 98 - RNA, 19 nt, Homo sapiens
aaaagcuggguugagagga

SEQ ID NO: 99 - RNA, 22 nt, Homo sapiens
uucaccaccuucuccacccagc

SEQ ID NO: 100 - RNA, 22 nt, Homo sapiens
aaaagcuggguugagagggcaa

SEQ ID NO: 101 - RNA, 22 nt, Homo sapiens
ugagaacugaauuccauaggcu

SEQ ID NO: 102 - RNA, 23 nt, Homo sapiens
uuuggcacuagcacauuuuugcu

SEQ ID NO: 103 - RNA, 22 nt, Homo sapiens
aaaagcuggguugagagggcga

SEQ ID NO: 104 - RNA, 23 nt, Homo sapiens
cggcccgggcugcugcuguuccu

SEQ ID NO: 105 - RNA, 22 nt, Homo sapiens
ugaguauuacauggccaaucuc

SEQ ID NO: 106 - RNA, 21 nt, Homo sapiens
uacaguacugugauaacugaa

SEQ ID NO: 107 - RNA, 22 nt, Homo sapiens
ucaccagcccuguguucccuag

SEQ ID NO: 108 - RNA, 22 nt, Homo sapiens
uauugcacauuacuaaguugca

SEQ ID NO: 109 - RNA, 22 nt, Homo sapiens
ugagguaguaguuuguacaguu

SEQ ID NO: 110 - RNA, 22 nt, Homo sapiens
uagcuuaucagacugauguuga

SEQ ID NO: 111 - RNA, 23 nt, Homo sapiens
caaagugcuguucgugcagguag

SEQ ID NO: 112 - RNA, 23 nt, Homo sapiens
cagugcaauaguauugucaaagc

SEQ ID NO: 113 - RNA, 21 nt, Homo sapiens
uucacaguggcuaaguucugc

SEQ ID NO: 114 - RNA, 23 nt, Homo sapiens
acuugggcacugaaacaaugucc

SEQ ID NO: 115 - RNA, 22 nt, Homo sapiens
acuccagccccacagccucagc

SEQ ID NO: 116 - RNA, 21 nt, Homo sapiens
uaaagugcugacagugcagau

SEQ ID NO: 117 - RNA, 20 nt, Homo sapiens
gugcauugcuguugcauugc

SEQ ID NO: 118 - RNA, 21 nt, Homo sapiens
agcuacaucuggcuacugggu

SEQ ID NO: 119 - RNA, 22 nt, Homo sapiens
aacacaccuauucaaggauuca

SEQ ID NO: 120 - RNA, 22 nt, Homo sapiens
ugucuacuacuggagacacugg

SEQ ID NO: 121 - RNA, 22 nt, Homo sapiens
caacuagacugugagcuucuag

SEQ ID NO: 122 - RNA, 22 nt, Homo sapiens
ccuauucuugauuacuuguuuc

SEQ ID NO: 123 - RNA, 23 nt, Homo sapiens
ugucugcccgcaugccugccucu

SEQ ID NO: 124 - RNA, 22 nt, Homo sapiens
ugguugaccauagaacaugcgc

SEQ ID NO: 125 - RNA, 22 nt, Homo sapiens
auaagacgaacaaaagguuugu

SEQ ID NO: 126 - RNA, 22 nt, Homo sapiens
acaguagucugcacauugguua

SEQ ID NO: 127 - RNA, 21 nt, Homo sapiens
aaggcagggcccccgcucccc

SEQ ID NO: 128 - RNA, 23 nt, Homo sapiens
ucucacacagaaaucgcacccgu

SEQ ID NO: 129 - RNA, 22 nt, Homo sapiens
caucuuccaguacaguguugga

SEQ ID NO: 130 - RNA, 22 nt, Homo sapiens
uuuucaacucuaaugggagaga

SEQ ID NO: 131 - RNA, 20 nt, Homo sapiens
ucacuguucagacaggcgga

SEQ ID NO: 132 - RNA, 22 nt, Homo sapiens
uccgagccugggucucccucuu

SEQ ID NO: 133 - RNA, 22 nt, Homo sapiens
uuguacaugguaggcuuucauu

SEQ ID NO: 134 - RNA, 19 nt, Homo sapiens
aggcugcggaauucaggac

SEQ ID NO: 135 - RNA, 22 nt, Homo sapiens
gcuauuucacgacaccaggguu

SEQ ID NO: 136 - RNA, 22 nt, Homo sapiens
accguggcuuucgauuguuacu

SEQ ID NO: 137 - RNA, 21 nt, Homo sapiens
gaaggcgcuucccuuuggagu

SEQ ID NO: 138 - RNA, 21 nt, Homo sapiens
uugaaaggcuauuucuugguc

SEQ ID NO: 139 - RNA, 21 nt, Homo sapiens
aaagugcuuccuuuuugaggg

SEQ ID NO: 140 - RNA, 23 nt, Homo sapiens
uaaauuucaccuuucugagaagg

SEQ ID NO: 141 - RNA, 22 nt, Homo sapiens
ggcuacaacacaggacccgggc

SEQ ID NO: 142 - RNA, 22 nt, Homo sapiens
aggcggggcgccgcgggaccgc

SEQ ID NO: 143 - RNA, 22 nt, Homo sapiens
aaaaguacuugcggauuuugcu

SEQ ID NO: 144 - RNA, 22 nt, Homo sapiens
gaaguuguucgugguggauucg

SEQ ID NO: 145 - RNA, 22 nt, Homo sapiens
acgcccuucccccccuucuuca

SEQ ID NO: 146 - RNA, 21 nt, Homo sapiens
aggcggagacuugggcaauug

SEQ ID NO: 147 - RNA, 22 nt, Homo sapiens
cguguucacagcggaccuugau

SEQ ID NO: 148 - RNA, 22 nt, Homo sapiens
cagugcaauguuaaaagggcau

SEQ ID NO: 149 - RNA, 23 nt, Homo sapiens
aaugacacgaucacucccguuga

SEQ ID NO: 150 - RNA, 19 nt, Homo sapiens
ugagcugcuguaccaaaau

SEQ ID NO: 151 - RNA, 22 nt, Homo sapiens
cggaugagcaaagaaagugguu

SEQ ID NO: 152 - RNA, 22 nt, Homo sapiens
ggauuccuggaaauacuguucu

SEQ ID NO: 153 - RNA, 22 nt, Homo sapiens
ucuacaaaggaaagcgcuuucu

SEQ ID NO: 154 - RNA, 20 nt, Homo sapiens
ccauggaucuccaggugggu

SEQ ID NO: 155 - RNA, 21 nt, Homo sapiens
uguucauguagauguuuaagc

SEQ ID NO: 156 - RNA, 22 nt, Homo sapiens
aagugcugucauagcugagguc

SEQ ID NO: 157 - RNA, 22 nt, Homo sapiens
cugggagguggauguuuacuuc

SEQ ID NO: 158 - RNA, 21 nt, Homo sapiens
uaccacaggguagaaccacgg

SEQ ID NO: 159 - RNA, 22 nt, Homo sapiens
ugugacugguugaccagagggg

SEQ ID NO: 160 - RNA, 22 nt, Homo sapiens
uucaaguaauccaggauaggcu

SEQ ID NO: 161 - RNA, 21 nt, Homo sapiens
cggcggggacggcgauugguc

SEQ ID NO: 162 - RNA, 22 nt, Homo sapiens
acaggugagguucuugggagcc

SEQ ID NO: 163 - RNA, 22 nt, Homo sapiens
ucagcaaacauuuauugugugc

SEQ ID NO: 164 - RNA, 20 nt, Homo sapiens
ccccagggcgacgcggcggg

SEQ ID NO: 165 - RNA, 21 nt, Homo sapiens
cacaagguauugguauuaccu

SEQ ID NO: 166 - RNA, 22 nt, Homo sapiens
uggagugugacaaugguguuug

SEQ ID NO: 167 - RNA, 22 nt, Homo sapiens
uggguggucuggagauuugugc

SEQ ID NO: 168 - RNA, 21 nt, Homo sapiens
cuagacugaagcuccuugagg

SEQ ID NO: 169 - RNA, 21 nt, Homo sapiens
cccggagccaggaugcagcuc

SEQ ID NO: 170 - RNA, 22 nt, Homo sapiens
ucaguaaauguuuauuagauga

SEQ ID NO: 171 - RNA, 23 nt, Homo sapiens
gacacgggcgacagcugcggccc

SEQ ID NO: 172 - RNA, 23 nt, Homo sapiens
gaagugcuucgauuuuggggugu

SEQ ID NO: 173 - RNA, 23 nt, Homo sapiens
aaacucuacuuguccuucugagu

SEQ ID NO: 174 - RNA, 22 nt, Homo sapiens
uagcagcacaucaugguuuaca

SEQ ID NO: 175 - RNA, 22 nt, Homo sapiens
gaugagcucauuguaauaugag

SEQ ID NO: 176 - RNA, 22 nt, Homo sapiens
aaagugcuuccuuuuagagggu

SEQ ID NO: 177 - RNA, 22 nt, Homo sapiens
gaggguuggguggaggcucucc

SEQ ID NO: 178 - RNA, 21 nt, Homo sapiens
uggacugcccugaucuggaga

SEQ ID NO: 179 - RNA, 21 nt, Homo sapiens
gcgacccauacuugguuucag

SEQ ID NO: 180 - RNA, 23 nt, Homo sapiens
caacggaaucccaaaagcagcug

SEQ ID NO: 181 - RNA, 22 nt, Homo sapiens
aaaaguaauugugguuuuugcc

SEQ ID NO: 182 - RNA, 22 nt, Homo sapiens
aaucacuaaccacacggccagg

SEQ ID NO: 183 - RNA, 21 nt, Homo sapiens
gugcauuguaguugcauugca

SEQ ID NO: 184 - RNA, 23 nt, Homo sapiens
uggaguccaggaaucugcauuuu

SEQ ID NO: 185 - RNA, 22 nt, Homo sapiens
uagcaaaaacugcaguuacuuu

SEQ ID NO: 186 - RNA, 21 nt, Homo sapiens
gcaguccaugggcauauacac

SEQ ID NO: 187 - RNA, 22 nt, Homo sapiens
auccgcgcucugacucucugcc

SEQ ID NO: 188 - RNA, 22 nt, Homo sapiens
agucauuggaggguuugagcag

SEQ ID NO: 189 - RNA, 22 nt, Homo sapiens
cuauacaaccuacugccuuccc

SEQ ID NO: 190 - RNA, 22 nt, Homo sapiens
gagcuuauucauaaaagugcag

SEQ ID NO: 191 - RNA, 22 nt, Homo sapiens
augcugacauauuuacuagagg

SEQ ID NO: 192 - RNA, 21 nt, Homo sapiens
aagugaucuaaaggccuacau

SEQ ID NO: 193 - RNA, 22 nt, Homo sapiens
uugcauagucacaaaagugauc

SEQ ID NO: 194 - RNA, 22 nt, Homo sapiens
uaguacugugcauaucaucuau

SEQ ID NO: 195 - RNA, 23 nt, Homo sapiens
ugugcaaaucuaugcaaaacuga

SEQ ID NO: 196 - RNA, 21 nt, Homo sapiens
caacaccagucgaugggcugu

SEQ ID NO: 197 - RNA, 22 nt, Homo sapiens
uauugcacucgucccggccucc

SEQ ID NO: 198 - RNA, 23 nt, Homo sapiens
ugauuguagccuuuuggaguaga

SEQ ID NO: 199 - RNA, 22 nt, Homo sapiens
ugcggggcuagggcuaacagca

SEQ ID NO: 200 - RNA, 19 nt, Homo sapiens
gggcgccugugaucccaac

SEQ ID NO: 201 - RNA, 22 nt, Homo sapiens
gaauguugcucggugaaccccu

SEQ ID NO: 202 - RNA, 22 nt, Homo sapiens
aacuggaucaauuauaggagug

SEQ ID NO: 203 - RNA, 23 nt, Homo sapiens
uuauugcuuaagaauacgcguag

SEQ ID NO: 204 - RNA, 21 nt, Homo sapiens
ugacaacuauggaugagcucu

SEQ ID NO: 205 - RNA, 22 nt, Homo sapiens
cugccaauuccauaggucacag

SEQ ID NO: 206 - RNA, 22 nt, Homo sapiens
ccucuagauggaagcacugucu

SEQ ID NO: 207 - RNA, 22 nt, Homo sapiens
ucuucucuguuuuggccaugug

SEQ ID NO: 208 - RNA, 23 nt, Homo sapiens
agcagcauuguacagggcuauga

SEQ ID NO: 209 - RNA, 22 nt, Homo sapiens
ugccugucuacacuugcugugc

SEQ ID NO: 210 - RNA, 23 nt, Homo sapiens
ccucagggcuguagaacagggcu

SEQ ID NO: 211 - RNA, 23 nt, Homo sapiens
guccaguuuucccaggaaucccu

SEQ ID NO: 212 - RNA, 22 nt, Homo sapiens
cucuagagggaagcgcuuucug

SEQ ID NO: 213 - RNA, 22 nt, Homo sapiens
cuauacgaccugcugccuuucu

SEQ ID NO: 214 - RNA, 22 nt, Homo sapiens
cuggcccucucugcccuuccgu

SEQ ID NO: 215 - RNA, 22 nt, Homo sapiens
aguggggaacccuuccaugagg

SEQ ID NO: 216 - RNA, 22 nt, Homo sapiens
ugcccuguggacucaguucugg

SEQ ID NO: 217 - RNA, 22 nt, Homo sapiens
cucggcgcggggcgcgggcucc

SEQ ID NO: 218 - RNA, 21 nt, Homo sapiens
cuguacaggccacugccuugc

SEQ ID NO: 219 - RNA, 21 nt, Homo sapiens
cacuguguccuuucugcguag

SEQ ID NO: 220 - RNA, 22 nt, Homo sapiens
auaaagcuagauaaccgaaagu

SEQ ID NO: 221 - RNA, 22 nt, Homo sapiens
uccauuacacuacccugccucu

SEQ ID NO: 222 - RNA, 22 nt, Homo sapiens
agggacgggacgcggugcagug

SEQ ID NO: 223 - RNA, 23 nt, Homo sapiens
cugggaucuccggggucuugguu

SEQ ID NO: 224 - RNA, 20 nt, Homo sapiens
cgugccacccuuuuccccag

SEQ ID NO: 225 - RNA, 22 nt, Homo sapiens
cucuagagggaagcgcuuucug

SEQ ID NO: 226 - RNA, 25 nt, Homo sapiens
cuagugagggacagaaccaggauuc

SEQ ID NO: 227 - RNA, 22 nt, Homo sapiens
gaaagcgcuucccuuugcugga

SEQ ID NO: 228 - RNA, 22 nt, Homo sapiens
caggccauauugugcugccuca

SEQ ID NO: 229 - RNA, 23 nt, Homo sapiens
ucauagcccuguacaaugcugcu

SEQ ID NO: 230 - RNA, 21 nt, Homo sapiens
ugagaugaagcacuguagcuc

SEQ ID NO: 231 - RNA, 21 nt, Homo sapiens
guguugaaacaaucucuacug

SEQ ID NO: 232 - RNA, 22 nt, Homo sapiens
acuggacuuagggucagaaggc

SEQ ID NO: 233 - RNA, 21 nt, Homo sapiens
guucaaauccagaucuauaac

SEQ ID NO: 234 - RNA, 22 nt, Homo sapiens
aggcaguguauuguuagcuggc

SEQ ID NO: 235 - RNA, 22 nt, Homo sapiens
acagauucgauucuaggggaau

SEQ ID NO: 236 - RNA, 26 nt, Homo sapiens
gaugaugauggcagcaaauucugaaa

SEQ ID NO: 237 - RNA, 21 nt, Homo sapiens
acucuagcugccaaaggcgcu

SEQ ID NO: 238 - RNA, 22 nt, Homo sapiens
uccagcaucagugauuuuguug

SEQ ID NO: 239 - RNA, 23 nt, Homo sapiens
cuggagauauggaagagcugugu

SEQ ID NO: 240 - RNA, 22 nt, Homo sapiens
aauugcacuuuagcaaugguga

SEQ ID NO: 241 - RNA, 22 nt, Homo sapiens
acugcagugaaggcacuuguag

SEQ ID NO: 242 - RNA, 22 nt, Homo sapiens
uuuugcgauguguuccuaauau

SEQ ID NO: 243 - RNA, 22 nt, Homo sapiens
cugaagcucagagggcucugau

SEQ ID NO: 244 - RNA, 21 nt, Homo sapiens
acucuuucccuguugcacuac

SEQ ID NO: 245 - RNA, 24 nt, Homo sapiens
ucccugagacccuuuaaccuguga

SEQ ID NO: 246 - RNA, 22 nt, Homo sapiens
ugagguaguagauuguauaguu

SEQ ID NO: 247 - RNA, 24 nt, Homo sapiens
acugggggcuuucgggcucugcgu

SEQ ID NO: 248 - RNA, 21 nt, Homo sapiens
gugggcgggggcaggugugug

SEQ ID NO: 249 - RNA, 21 nt, Homo sapiens
cucccacaugcaggguuugca

SEQ ID NO: 250 - RNA, 21 nt, Homo sapiens
agggggaaaguucuauagucc

SEQ ID NO: 251 - RNA, 21 nt, Homo sapiens
cauuauuacuuuugguacgcg

SEQ ID NO: 252 - RNA, 22 nt, Homo sapiens
uucucaaggaggugucguuuau

SEQ ID NO: 253 - RNA, 21 nt, Homo sapiens
ugaguuggccaucugagugag

SEQ ID NO: 254 - RNA, 21 nt, Homo sapiens
uagcagcacagaaauauuggc

SEQ ID NO: 255 - RNA, 24 nt, Homo sapiens
ucagaacaaaugccgguucccaga

SEQ ID NO: 256 - RNA, 23 nt, Homo sapiens
uauggcuuuuuauuccuauguga

SEQ ID NO: 257 - RNA, 22 nt, Homo sapiens
uuuuucauuauugcuccugacc

SEQ ID NO: 258 - RNA, 22 nt, Homo sapiens
uagcaccaucugaaaucgguua

SEQ ID NO: 259 - RNA, 23 nt, Homo sapiens
ugcaccaugguugucugagcaug

SEQ ID NO: 260 - RNA, 22 nt, Homo sapiens
uaacacugucugguaacgaugu

SEQ ID NO: 261 - RNA, 22 nt, Homo sapiens
aagauguggaaaaauuggaauc

SEQ ID NO: 262 - RNA, 19 nt, Homo sapiens
agcugucugaaaaugucuu

SEQ ID NO: 263 - RNA, 23 nt, Homo sapiens
uagcaccauuugaaaucaguguu

SEQ ID NO: 264 - RNA, 22 nt, Homo sapiens
uucccuuugucauccuaugccu

SEQ ID NO: 265 - RNA, 21 nt, Homo sapiens
gcgacccacucuugguuucca

SEQ ID NO: 266 - RNA, 22 nt, Homo sapiens
aaugcaccugggcaaggauuca

SEQ ID NO: 267 - RNA, 23 nt, Homo sapiens
uuacaguuguucaaccaguuacu

SEQ ID NO: 268 - RNA, 22 nt, Homo sapiens
ugaccgauuucuccugguguuc

SEQ ID NO: 269 - RNA, 22 nt, Homo sapiens
ugagguaguaguuugugcuguu

SEQ ID NO: 270 - RNA, 23 nt, Homo sapiens
ucccuguccuccaggagcucacg

SEQ ID NO: 271 - RNA, 22 nt, Homo sapiens
aagugccuccuuuuagaguguu

SEQ ID NO: 272 - RNA, 22 nt, Homo sapiens
ucaggcucaguccccucccgau

SEQ ID NO: 273 - RNA, 22 nt, Homo sapiens
uuugguccccuucaaccagcua

SEQ ID NO: 274 - RNA, 23 nt, Homo sapiens
ccuaguagguguccaguaagugu

SEQ ID NO: 275 - RNA, 22 nt, Homo sapiens
aucgugcaucccuuuagagugu

SEQ ID NO: 276 - RNA, 23 nt, Homo sapiens
ugaguaccgccaugucuguuggg

SEQ ID NO: 277 - RNA, 22 nt, Homo sapiens
aaccaucgaccguugaguggac

SEQ ID NO: 278 - RNA, 21 nt, Homo sapiens
ccgucgccgccacccgagccg

SEQ ID NO: 279 - RNA, 20 nt, Homo sapiens
auguauaaauguauacacac

SEQ ID NO: 280 - RNA, 22 nt, Homo sapiens
ugaaggucuacugugugccagg

SEQ ID NO: 281 - RNA, 22 nt, Homo sapiens
acugcugagcuagcacuucccg

SEQ ID NO: 282 - RNA, 19 nt, Homo sapiens
uggauuuuuggaucaggga

SEQ ID NO: 283 - RNA, 23 nt, Homo sapiens
cgcauccccuagggcauuggugu

SEQ ID NO: 284 - RNA, 23 nt, Homo sapiens
agcucggucugaggccccucagu

SEQ ID NO: 285 - RNA, 22 nt, Homo sapiens
ugcuaugccaacauauugccau

SEQ ID NO: 286 - RNA, 22 nt, Homo sapiens
ccuguucuccauuacuuggcuc

SEQ ID NO: 287 - RNA, 22 nt, Homo sapiens
uggcucaguucagcaggaacag

SEQ ID NO: 288 - RNA, 22 nt, Homo sapiens
caaaacuggcaauuacuuuugc

SEQ ID NO: 289 - RNA, 23 nt, Homo sapiens
uauucauuuauccccagccuaca

SEQ ID NO: 290 - RNA, 22 nt, Homo sapiens
cuauacggccuccuagcuuucc

SEQ ID NO: 291 - RNA, 21 nt, Homo sapiens
aaaacggugagauuuuguuuu

SEQ ID NO: 292 - RNA, 22 nt, Homo sapiens
cgucaacacuugcugguuuccu

SEQ ID NO: 293 - RNA, 22 nt, Homo sapiens
aucgggaaugucguguccgccc

SEQ ID NO: 294 - RNA, 22 nt, Homo sapiens
uuccuaugcauauacuucuuug

SEQ ID NO: 295 - RNA, 23 nt, Homo sapiens
uaagugcuuccauguuugagugu

SEQ ID NO: 296 - RNA, 22 nt, Homo sapiens
caaaaaucucaauuacuuuugc

SEQ ID NO: 297 - RNA, 23 nt, Homo sapiens
ucuggcuccgugucuucacuccc

SEQ ID NO: 298 - RNA, 22 nt, Homo sapiens
agggcuuagcugcuugugagca

SEQ ID NO: 299 - RNA, 21 nt, Homo sapiens
uacucaaaaagcugucaguca

SEQ ID NO: 300 - RNA, 22 nt, Homo sapiens
aaaaguaauugcggauuuugcc

SEQ ID NO: 301 - RNA, 22 nt, Homo sapiens
uaacugguugaacaacugaacc

SEQ ID NO: 302 - RNA, 22 nt, Homo sapiens
cuauacagucuacugucuuucc

SEQ ID NO: 303 - RNA, 22 nt, Homo sapiens
uauacaagggcaagcucucugu

SEQ ID NO: 304 - RNA, 25 nt, Homo sapiens
ggcggagggaaguagguccguuggu

SEQ ID NO: 305 - RNA, 24 nt, Homo sapiens
agcagaagcagggagguucuccca

SEQ ID NO: 306 - RNA, 22 nt, Homo sapiens
cugguacaggccugggggacag

SEQ ID NO: 307 - RNA, 22 nt, Homo sapiens
ugaaacauacacgggaaaccuc

SEQ ID NO: 308 - RNA, 21 nt, Homo sapiens
ccuggaaacacugagguugug

SEQ ID NO: 309 - RNA, 25 nt, Homo sapiens
agggaucgcgggcggguggcggccu

SEQ ID NO: 310 - RNA, 21 nt, Homo sapiens
uggguuuacguugggagaacu

SEQ ID NO: 311 - RNA, 22 nt, Homo sapiens
gacugacaccucuuugggugaa

SEQ ID NO: 312 - RNA, 22 nt, Homo sapiens
aaaaguaauugcggucuuuggu

SEQ ID NO: 313 - RNA, 21 nt, Homo sapiens
gcuaguccugacucagccagu

SEQ ID NO: 314 - RNA, 21 nt, Homo sapiens
ucccacguuguggcccagcag

SEQ ID NO: 315 - RNA, 22 nt, Homo sapiens
cuccugagccauucugagccuc

SEQ ID NO: 316 - RNA, 22 nt, Homo sapiens
aaaaguaaucgcgguuuuuguc

SEQ ID NO: 317 - RNA, 22 nt, Homo sapiens
ccaguggggcugcuguuaucug

SEQ ID NO: 318 - RNA, 22 nt, Homo sapiens
ugagguaguagguuguauaguu

SEQ ID NO: 319 - RNA, 22 nt, Homo sapiens
gaaaucaagcgugggugagacc

SEQ ID NO: 320 - RNA, 22 nt, Homo sapiens
ggaggggucccgcacugggagg

SEQ ID NO: 321 - RNA, 22 nt, Homo sapiens
aagcccuuaccccaaaaaguau

SEQ ID NO: 322 - RNA, 22 nt, Homo sapiens
gggagccaggaaguauugaugu

SEQ ID NO: 323 - RNA, 21 nt, Homo sapiens
aauggcgccacuaggguugug

SEQ ID NO: 324 - RNA, 22 nt, Homo sapiens
uuuaacauggggguaccugcug

SEQ ID NO: 325 - RNA, 21 nt, Homo sapiens
ucagugcaugacagaacuugg

SEQ ID NO: 326 - RNA, 22 nt, Homo sapiens
ggagaaauuauccuuggugugu

SEQ ID NO: 327 - RNA, 22 nt, Homo sapiens
ugauauguuugauauauuaggu

SEQ ID NO: 328 - RNA, 22 nt, Homo sapiens
cugcccuggcccgagggaccga

SEQ ID NO: 329 - RNA, 21 nt, Homo sapiens
ugagugccggugccugcccug

SEQ ID NO: 330 - RNA, 22 nt, Homo sapiens
uagguaguuucauguuguuggg

SEQ ID NO: 331 - RNA, 22 nt, Homo sapiens
uguaacagcaacuccaugugga

SEQ ID NO: 332 - RNA, 21 nt, Homo sapiens
auccuugcuaucugggugcua

SEQ ID NO: 333 - RNA, 21 nt, Homo sapiens
caaagaggaaggucccauuac

SEQ ID NO: 334 - RNA, 22 nt, Homo sapiens
gugacaucacauauacggcagc

SEQ ID NO: 335 - RNA, 22 nt, Homo sapiens
ugagaccucuggguucugagcu

SEQ ID NO: 336 - RNA, 20 nt, Homo sapiens
ugagcccuguccucccgcag

SEQ ID NO: 337 - RNA, 21 nt, Homo sapiens
uuuugcaccuuuuggagugaa

SEQ ID NO: 338 - RNA, 21 nt, Homo sapiens
aacauagaggaaauuccacgu

SEQ ID NO: 339 - RNA, 22 nt, Homo sapiens
aaacaaacauggugcacuucuu

SEQ ID NO: 340 - RNA, 24 nt, Homo sapiens
ccagacagaauucuaugcacuuuc

SEQ ID NO: 341 - RNA, 21 nt, Homo sapiens
ccaguccugugccugccgccu

SEQ ID NO: 342 - RNA, 22 nt, Homo sapiens
auucuaauuucuccacgucuuu

SEQ ID NO: 343 - RNA, 24 nt, Homo sapiens
agccuggaagcuggagccugcagu

SEQ ID NO: 344 - RNA, 21 nt, Homo sapiens
uugcucacuguucuucccuag

SEQ ID NO: 345 - RNA, 22 nt, Homo sapiens
caacaaaucccagucuaccuaa

SEQ ID NO: 346 - RNA, 21 nt, Homo sapiens
caagucacuagugguuccguu

SEQ ID NO: 347 - RNA, 22 nt, Homo sapiens
cgcaggggccgggugcucaccg

SEQ ID NO: 348 - RNA, 20 nt, Homo sapiens
caccaggcauuguggucucc

SEQ ID NO: 349 - RNA, 22 nt, Homo sapiens
aaugcacccgggcaaggauucu

SEQ ID NO: 350 - RNA, 23 nt, Homo sapiens
uauggcuuuucauuccuauguga

SEQ ID NO: 351 - RNA, 23 nt, Homo sapiens
aaggagcuuacaaucuagcuggg

SEQ ID NO: 352 - RNA, 22 nt, Homo sapiens
cucuagagggaagcgcuuucug

SEQ ID NO: 353 - RNA, 24 nt, Homo sapiens
aauccuuggaaccuaggugugagu